(19) United States
(12) Patent Application Publication
(10) Pub. No.: US 2026/0055402 A1
(43) Pub. Date: Feb. 26, 2026

(54) OLIGONUCLEOTIDE COMPOSITIONS AND METHODS THEREOF

(71) Applicant: WAVE LIFE SCIENCES LTD., Singapore (SG)

(72) Inventors: Stephen Lister Lake, Newton, MA (US); Xiao Shelley Hu, Brookline, MA (US); Jianxin Gao, Norton, MA (US); Yilin Zhang, West Roxbury, MA (US); Philip Ross, Milford, MA (US); Vincent Aduda, Acton, MA (US); Mamoru Shimizu, Arlington, MA (US); Danlin Xu, Summit, NJ (US); Keith Andrew Bowman, Stow, MA (US); Sarah Diane Lamore, Burlington, MA (US); Michael Angelo Panzara, Boston, MA (US); Mohammed Rowshon Alam, Hopkinton, MA (US); Hyun Gyung Jang, Waltham, MA (US); Anne-Marie Li-Kwai-Cheung, Dorridge, Solihull (GB); Padmakumar Narayanan, Carlsbad, CA (US)

(21) Appl. No.: 19/102,669

(22) PCT Filed: Aug. 11, 2023
(86) PCT No.: PCT/US2023/030103

§ 371 Date: Feb. 10, 2025

Related U.S. Application Data
(60) Provisional application No. 63/397,284, filed on Aug. 11, 2022.
(60) Provisional application No. 63/408,427, filed on Sep. 20, 2022.
(60) Provisional application No. 63/517,529, filed on Aug. 3, 2023.

(51) Int. Cl. C12N 15/113

(52) U.S. Cl. CPC C12N 15/113; C12N 2310/14; C12N 2310/315; C12N 2310/321; C12N 2310/3341

(57) ABSTRACT

Among other things, the present disclosure provides various oligonucleotide technologies including chirally controlled oligonucleotide compositions and technologies for manufacturing such oligonucleotide compositions. In some embodiments, a method is a method of treatment or prevention of Huntington's Disease in a subject in need thereof; a method of allele-specific knockdown of a mutant Huntingtin transcript in a subject; a method for delaying the onset of and/or reducing the severity of at least one symptom of Huntington's Disease in a subject with Huntington's Disease; a method of reducing the expression, level, amount and/or activity of a mutant Huntingtin gene or a gene product thereof; and/or a method of preparation of a medicament for treatment of Huntington's Disease, wherein the method pertains to the use of an oligonucleotide described herein, administered at a dose described herein. In some embodiments, the present disclosure provides doses, dosages, and formulations of an oligonucleotide described herein.

### CROSS-REFERENCE TO RELATED APPLICATIONS

This application claims priority to United States Provisional Application Nos. 63/397,284, filed Aug. 11, 2022, 63/408,427, filed Sep. 20, 2022, and 63/517,529, filed Aug. 3, 2023, the entirety of each of which is incorporated herein by reference.

### FIELD

Among other things, the present disclosure provides technologies for treating Huntington's disease. In some embodiments, provided herein are methods of administering WVE-003 for ameliorating Huntington's disease, reducing mHTT RNA, or reducing mHTT protein in a human subject in need thereof. In certain instances, methods are useful for ameliorating at least one symptom of Huntington's disease. Such symptoms of Huntington's disease include, but are not limited to, brain atrophy, muscle atrophy, nerve degeneration, uncontrolled movement, difficulty swallowing, difficulty speaking, anxiety and depression.

### BACKGROUND

Oligonucleotides are useful in therapeutic, diagnostic, research and nanomaterials applications. The use of naturally occurring nucleic acids (e.g., unmodified DNA or RNA) for therapeutics can be limited, for example, because of their instability against extra- and intracellular nucleases and/or their poor cell penetration and distribution. Currently lacking are acceptable options for treating neurodegenerative diseases like Huntington's disease. It is an object herein to provide methods for the treatment of such diseases.

### SUMMARY

WVE-003, which may also be referred to as WV-21405, has the structure of mG*S mUn001R mU mGn001RmA*ST*SC*ST*SG*ST*RA*SG*SC*SA*SG*Rm5Ceon001RAeoGeon001R m5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

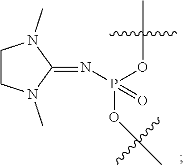 [SMILES: *OP(=O)(N=C1N(C)CCN1C)O*]

eo represents a 2′-OCH2CH2OCH3 modification to a nucleoside; and
*R represents a Rp phosphorothioate linkage.
   As typically utilized in oligonucleotide description, if not specifically indicated a nucleoside is a DNA nucleoside and a linkage is a natural phosphate linkage.

As described herein, WVE-003 can be provided in various forms including pharmaceutically acceptable salt forms such as sodium salt.

Among other things, the present disclosure encompasses the recognition that structural elements of HTT (Huntingtin) oligonucleotides, such as base sequence, chemical modifications (e.g., modifications of sugar, base, and/or internucleotidic linkages, and patterns thereof), and/or stereochemistry [e.g., stereochemistry of backbone chiral centers (chiral internucleotidic linkages), and/or patterns thereof], can have a significant impact on HTT oligonucleotide properties and/or activities, e.g., protein binding characteristics, stability, toxicities, delivery, allele-specific knockdown of HTT transcripts, etc. In some embodiments, the present disclosure demonstrates that HTT oligonucleotides with controlled structural elements and compositions thereof can provide unexpected therapeutic properties and activities. In some embodiments, the present disclosure provides technologies, e.g., HTT oligonucleotides, pharmaceutical compositions, drug products, doses, dosages, etc., particularly those of WVE-003, that are useful for treating Huntington's disease in a subject. In some embodiments, a subject has a HTT allele or transcript that comprises an expanded CAG repeat region and is fully complementary to the base sequence of WVE-003. As used herein, a nucleic acid or base sequence may be considered as fully complementary to another nucleic acid (e.g., WVE-003) or base sequence if the nucleic acid or base sequence comprises a sequence that has the same length as the another nucleic acid or base sequence and is fully complementary to the full base sequence of the another nucleic acid (e.g., WVE-003) or base sequence. In some embodiments, an expanded CAG repeat comprises 36 or more CAG repeats. In some embodiments, a subject comprises a HTT transcript associated with Huntington's disease, wherein the transcript is fully complementary to the base sequence of WVE-003. In some embodiments, a subject comprises a HTT transcript associated with Huntington's disease, wherein the transcript is fully complementary to the base sequence of WVE-003 at SNP rs362273. In some embodiments, a subject comprises a HTT transcript that is fully complementary to the base sequence of WVE-003 and comprises an expanded CAG repeat region. In some embodiments, a subject comprises a HTT transcript that is fully complementary to the base sequence of WVE-003 at SNP rs362273 and comprises an expanded CAG repeat region. In some embodiments, a subject comprises a HTT nucleic acid, e.g., a HTT gene, which encodes a HTT transcript associated with Huntington's disease, wherein the transcript is fully complementary to the base sequence of WVE-003. In some embodiments, a subject comprises a HTT nucleic acid, e.g., a HTT gene, which encodes a HTT transcript associated with Huntington's disease, wherein the transcript is fully complementary to the base sequence of WVE-003 at SNP rs362273. In some embodiments, a subject comprises a HTT nucleic acid, e.g., a HTT gene, which encodes a HTT transcript that is fully complementary to the base sequence of WVE-003 and comprises an expanded CAG repeat region. In some embodiments, a subject comprises a HTT nucleic acid, e.g., a HTT gene, which encodes a HTT transcript that is fully complementary to the base sequence of WVE-003 at SNP rs362273 and comprises an expanded CAG repeat region. In some embodiments, a subject has an A variant of SNP rs362273. As those skilled in the art appreciate, HTT transcripts with an A variant of SNP rs362273 can be fully complementary to the base sequence of WVE-003, wherein the A is complementary to a T in WVE-003. In some embodiments, the A variant of SNP rs362273 is on the same chromosome as an expanded CAG repeat region in HTT. In some embodiments, a subject has a variant of SNP rs362273 that is not A. In some embodiments, a variant of SNP rs362273 that is not A is not on the same chromosome as an expanded CAG repeat region in HTT. In some embodiments, a variant of SNP rs362273 is C. In some embodiments, a subject is heterozygous for the A variant of SNP rs362273. In some embodiments, a subject is heterozygous for the A variant of SNP rs362273, wherein the A variant is on the same chromosome as an expanded CAG repeat region in HTT. In some embodiments, a subject is homozygous for the A variant of SNP rs362273. In some embodiments, a subject is homozygous for the A variant of SNP rs362273 and has an expanded CAG repeat region in HTT. In some embodiments, one chromosome of a subject has an expanded CAG repeat region in HTT. In some embodiments, both chromosomes of a subject independently have an expanded CAG repeat region in HTT.

In some embodiments, Huntington's disease (HD) is a debilitating and ultimately fatal autosomal dominant neurological disorder, characterized by cognitive decline, psychiatric illness and chorea. In some embodiments, HD causes nerve cells in the brain to deteriorate over time, affecting thinking ability, emotions and movement. HD is reported to be caused by an expanded cytosine-adenine-guanine (CAG) triplet repeat in the huntingtin (HTT) gene that results in production of mutant HTT protein. In some embodiments, accumulation of mutant HTT causes progressive loss of neurons in the brain. In some embodiments, wild-type, or healthy, HTT protein is critical for neuronal function, and suppression may have detrimental long-term consequences. In some embodiments, approximately 30,000 people in the United States have symptomatic HD and more than 200,000 others are at risk for inheriting the disease. There are currently no approved disease-modifying therapies available.

In some embodiments, Huntington's disease (HD) is reportedly caused by a cytosine-adenine-guanine (CAG) repeat expansion in the Huntingtin (HTT) gene, resulting in mutant HTT (mHTT) protein production. In some embodiments, while mHTT is reportedly causative for disease, preclinical studies suggest loss of wild-type HTT (wtHTT) may also contribute to neuronal impairment. In some embodiments, wtHTT protein is reportedly critical for neuronal function; suppression of wtHTT may have detrimental long-term consequences.

In some embodiments, the ability to selectively lower production of mHTT protein while retaining a sufficient wtHTT level (also described as allele-specific knockdown) reportedly holds great promise for HD treatment. In some embodiments, as confirmed herein, oligonucleotide administrations as described herein can reduce level, expression and/or activity of mHTT transcripts and/or proteins more than those of wtHTT transcripts and/or proteins in individual subjects and/or populations of subjects.

In some embodiments, allele-specific knockdown utilizes an association between single nucleotide polymorphisms (SNPs) and genetic mutations to specifically target errors in genetic disorders, including Huntington's disease (HD).

In some embodiments, the present disclosure provides oligonucleotides, compositions and methods for allele-specific knockdown of a mHTT transcript, wherein allele-specific knockdown (also referenced as allele-specific suppression, allele-selective approach, allele-selective knockdown or suppression, or the like) preferentially decreases the level, expression and/or activity of a mHTT transcript (e.g., comprising an expanded CAG repeat region) and/or a product thereof (e.g., mHTT protein) relative to a wild-type HTT and/or a product thereof (e.g., wild-type HTT protein) (e.g., the expression, level and/or activity of wild-type HTT is not significantly decreased, not decreased, remains the same, or increases).

In some embodiments, a decrease in the level, expression and/or activity of a mHTT transcript results in or is associated with a decrease in the level, expression and/or activity of a mHTT protein, including but not limited to the formation, number and/or size of aggregates (coagula) of mHTT proteins. In some embodiments, a mHTT protein comprises an extended poly-glutamine (poly-Q) tract, e.g., translated from a CAG repeat expansion.

In some embodiments, the present disclosure pertains to: a method of treatment and/or prevention of Huntington's disease in a subject (e.g., a patient such as a human patient) in need thereof; a method of allele-specific knockdown of a mutant Huntingtin transcript in a subject; and/or a method for reducing the severity of and/or delaying the onset of one or more symptoms of Huntington's disease. In some embodiments, the method comprises the step of administering to the subject a therapeutically effective amount of a HTT oligonucleotide or HTT oligonucleotide composition. In some embodiments, the present disclosure pertains to: a method of treating, ameliorating, or slowing the onset or progression of Huntington's Disease, comprising administering to a subject a compound comprising a HTT oligonucleotide or HTT oligonucleotide composition, wherein the oligonucleotide is complementary to a mutant huntingtin allele at a position on the allele comprising a single nucleotide polymorphism (SNP) site, wherein the compound administered to the subject treats, ameliorates and/or slows the onset and/or progression Huntington's Disease by selectively reducing the mutant huntingtin allele. In some embodiments, the present disclosure pertains to: a method of ameliorating a symptom of Huntington's disease, comprising administering a HTT oligonucleotide or HTT oligonucleotide composition to human subject in need thereof.

In some embodiments, a HTT oligonucleotide is WVE-003. In some embodiments, a HTT oligonucleotide composition is a composition comprising WVE-003. In some embodiments, a WVE-003 composition is a chirally controlled oligonucleotide composition.

In some embodiments, WVE-003 compositions are chirally controlled (e.g., stereopure) HTT oligonucleotide composition capable of mediating an allele-specific decrease in the level, expression and/or activity of a mHTT transcript. WVE-003 can selectively targets one isoform of HTT SNP rs362273 (sometimes referenced as SNP3). WVE-003 is fully complementary to the sequence of the SNP (and surrounding sequences) that it is targeting. As described herein, in various embodiments, WVE-003 is fully complementary to a mtHTT transcript. In some embodiments, WVE-003 is fully complementary to a mtHTT transcript, and is not fully complementary to a wtHTT transcript. In some embodiments, a mtHTT transcript is associated with, or more associated with than a wtHTT transcript, a condition, disorder or disease like Huntington's disease. In some embodiments, a mtHTT comprises 36, 37, 38, 39, 40, 45, 50 or more CAG repeats. As appreciated by those skilled in the art, complementary to a transcript may be assessed using genetic sequences from which a transcript is transcribed.

In some embodiments, WVE-003 can be used as a disease-modifying agent for the treatment of subjects with Huntington's disease (HD). It can be provided as a stereopure antisense oligonucleotide (ASO) that selectively target the mutant form of the huntingtin (mHTT) gene transcript. In some embodiments, a composition of WVE-003 is a stereopure oligonucleotide composition which recognizes the disease-associated (e.g., mutant) allele of SNP rs362273 in the Huntingtin gene, is efficacious in reducing the level, expression and/or activity of a mHTT gene (or a gene product thereof), and is capable of mediating allele-specific knockdown of the mutant Huntingtin (mHTT) gene.

In some embodiments, the present disclosure provides methods for reducing level, expression and/or activity of a mHTT gene (or a gene product thereof), comprising administering to a subject an oligonucleotide at a dose or according to a dosage regimen as described herein. In some embodiments, level of mHTT transcripts is reduced. In some embodiments, level of mHTT proteins is reduced. In some embodiments, level, expression and/or activity of a wtHTT gene is not significantly reduced. In some embodiments, assessments are performed using a sample from a subject. In some embodiments, a subject in various methods herein is a human. In some embodiments, assessments are performed using a sample from a human subject who is susceptible to or suffering from a condition, disorder or disease associated with mutant HTT (e.g., Huntington's disease). In some embodiments, assessments are performed using a patient sample. A “patient sample” is any biological specimen from a patient. The term sample includes, but is not limited to, biological fluids such as blood, serum, plasma, urine, cerebrospinal fluid (CFS), tears, saliva, lymph, dialysis fluid, lavage fluid, semen, and/or other liquid samples, as well as cells and tissues of biological origin. In some preferred embodiments, assessments are performed using CSF samples. In some embodiments, assessments are performed using plasma samples. In some embodiments, assessments are performed using blood samples. In some embodiments, assessment are performed at suitable time point(s) as appreciated by those skilled in the art, e.g., after about 1, 2, 3, 4, 5, 6, 7, 8, 9, 10, 11, 12, 13, 14, 15, 16, 17, 18, 19, or 20 days, or about 3, 4, 5, 6, 7, or 8 weeks, or about 3, 4, 5, or 6 or more months after administration of a dose and before a next dose, if any, is administered. In some embodiments, assessment is performed after about 1, 2, 3, 4, 5, 6, 7, 8, 9, 10, 11, 12, 13, 14, 15, 16, 17, 18, 19, or 20 doses are administered. In some embodiments, assessment results are for an individual subject. In some embodiments, assessment are performed on a population of subjects, e.g., as typically performed in clinical trials or in clinical applications. In some embodiments, a population has a size of about 10, 20, 30, 40, 50, 60, 70, 80, 90, 100, 200, 500, 1000 or more subjects. In some embodiments, the size is about 10 or more subjects. In some embodiments, the size is about 20 or more subjects. In some embodiments, the size is about 36 subjects. In some embodiments, the size is about 30 or more subjects. In some embodiments, the size is about 40 or more subjects. In some embodiments, the size is about 50 or more subjects. In some embodiments, the size is about 100 or more subjects. In some embodiments, the size is about 200 or more subjects. In some embodiments, the size is about 500 or more subjects. In some embodiments, the size is about 1000 or more subjects. In some embodiments, reduction is about 10%, 20%, 25%, 30%, 40%, 50%, 60%, 70%, 80%, or 90% or more. In some embodiments, it is at least about 10% or more. In some embodiments, it is at least about 12% or more. In some embodiments, it is at least about 20% or more. In some embodiments, it is at least about 30% or more. In some embodiments, it is at least about 40% or more. In some embodiments, it is at least about 50% or more. As appreciated by those skilled in the art, in some embodiments, a mHTT gene or product thereof comprises a sequence that is the same or fully complementary to WVE-003 sequence and contains expanded CAG repeats (or a sequence encoded thereby), and a wild type HTT gene or product thereof does not contain a sequence that is the same or fully complementary to WVE-003 sequence in HTT or at rs362273 and contains fewer CAG repeats (e.g., not considered as expanded CAG repeats) (or a sequence encoded by the repeats).

In some embodiments, the present disclosure pertains to any of various methods related to the use of a HTT oligonucleotide or composition. In some embodiments, a HTT oligonucleotide is WVE-003. In some embodiments, a composition is a composition comprising WVE-003. In some embodiments, a composition is a WVE-003 composition and substantially all oligonucleotides in the composition are WVE-003 (as appreciated by those skilled in the art, some other oligonucleotides may exist as impurities, levels of which can and are typically controlled in provided compositions).

In some embodiments, a HTT oligonucleotide, e.g., WVE-003, or composition is administered or delivered to a subject at a dose of about 10-200 mg, about 10-190 mg, about 10-180 mg, about 10-170 mg, about 10-160 mg, about 10-150 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg.

As used herein, an amount of the present disclosure, e.g., about 10-200 mg, about 10-190 mg, about 10-180 mg, 10-170 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg, is typically equivalent to free acid form of an oligonucleotide, unless explicitly noted otherwise. In some embodiments, WVE-003 is administered or delivered to a subject at a dose equivalent to about 10-200 mg, about 10-190 mg, about 10-180 mg, about 10-170 mg, about 10-160 mg, about 10-150 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg WVE-003 free acid form. In some embodiments, a dose is equivalent to about 10 mg WVE-003 free acid form. In some embodiments, a dose is equivalent to about 20 mg WVE-003 free acid form. In some embodiments, a dose is equivalent to about 30 mg WVE-003 free acid form. In some embodiments, a dose is equivalent to about 60 mg WVE-003 free acid form. In some embodiments, a dose is equivalent to about 90 mg WVE-003 free acid form. In some embodiments, a dose is equivalent to about 120 mg WVE-003 free acid form. In some embodiments, a dose is equivalent to about 150 mg WVE-003 free acid fonn.

In some embodiments, a HTT oligonucleotide or composition is administered or delivered to a subject at a dose of about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg. In some embodiments, WVE-003 is administered or delivered to a subject at a dose equivalent to about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg WVE-003 free acid form. As described herein, WVE-003 may be administered or delivered in various forms including various pharmaceutically acceptable salt forms. In some embodiments, a composition administered to a subject comprises one or more forms of WVE-003, e.g., one or more pharmaceutically acceptable salt forms. In some embodiments, a composition is a liquid composition. In some embodiments, WVE-003 is dissolved in a suitable liquid, e.g., water or a suitable buffer as described herein such as buffered saline, cerebrospinal fluid (CSF), artificial cerebrospinal fluid (aCSF), etc.

Useful technologies for preparing oligonucleotides and products thereof, e.g., of WVE-003 are as described herein. In some embodiments, oligonucleotides, e.g., WVE-003 are administered as chirally controlled oligonucleotide compositions as described herein. In some embodiments, each chiral internucleotidic linkages independently has a stereopurity of about 97%, 98%, 99% or more. In some embodiments, stereopurity of an oligonucleotide, e.g., WVE-003, in a composition can be expressed by the product of multiplying stereopurity (or stereoselectivity) at each chiral internucleotidic linkage. In some embodiments, stereopurity/stereoselectivity of a chiral internucleotidic linkage can be assessed by stereopurity/stereoselectivity during formation of a corresponding dimer which comprises the chiral internucleotidic linkage and the two nucleosides it is bonded to. In some embodiments, stereopurity/stereoselectivity of a chiral internucleotidic linkage can be assessed by stereopurity/stereoselectivity during oligonucleotide synthesis when the chiral internucleotidic linkage is formed (e.g., an assessment of a product after the formation of the chiral internucleotidic linkage but before the formation of the next internucleotidic linkage). In some embodiments, stereopurity/stereoselectivity of a chiral internucleotidic linkage in a product oligonucleotide, e.g., WVE-003, is considered to be the stereopurity/stereoselectivity observed in dimer formations or during oligonucleotide synthesis. In some embodiments, stereopurity/stereoselectivity observed in dimer formations or during oligonucleotide synthesis are the same or very close to each other. Those skilled in the art appreciate that stereopurity/stereoselectivity of each chiral internucleotidic linkage may be different, though according to various provided technologies it is consistently high at each chiral internucleotidic linkage (e.g., about 97%, 98%, 99% or more). In some embodiments, each chiral internucleotidic linkage independently has a stereopurity of about 97%, 98%, 99% or more. In some embodiments, at least 1, 2, 3, 4, 5, 6, 7, 8, 9, 10, 11, 12, 13, 14, or 15 internucleotidic linkage independently has a stereopurity of about 98%, 99% or more. In some embodiments, at least 1, 2, 3, 4, 5, 6, or 7 internucleotidic linkage independently has a stereopurity of about 99% or more. In some embodiments, no more than 1, 2, 3, 4, or 5 independently has a stereopurity of less than about 98%. In some embodiments, no more than 1, 2, 3, 4, or 5 independently has a stereopurity of less than about 98% but each is still independently higher than about 97%. In some embodiments, stereopurity of WVE-003 is about 75% or more. In some embodiments, stereopurity of WVE-003 is about 80% or more (e.g., as product of stereopurity of corresponding dimers). In some embodiments, stereopurity of WVE-003 is about 81% or more (e.g., as product of stereopurity of corresponding dimers). In some embodiments, stereopurity of WVE-003 is about 82% or more (e.g., as product of stereopurity of corresponding dimers). In some embodiments, stereopurity of WVE-003 is about 83% or more (e.g., as product of stereopurity of corresponding dimers). In some embodiments, stereopurity of WVE-003 is about 84% or more (e.g., as product of stereopurity of corresponding dimers). In some embodiments, stereopurity of WVE-003 is about 85% or more (e.g., as product of stereopurity of corresponding dimers). In some embodiments, amount of an oligonucleotide administered include amount of stereoisomers of the oligonucleotide (e.g., to the extent cannot be separated during manufacturing). In some embodiments, amount of an oligonucleotide administered does not include other impurities such as shorter oligonucleotides observed during oligonucleotide synthesis (as appreciated by those skilled in the art, to the extent that a utilized purification method can remove such impurities).

As appreciated by those skilled in the art, oligonucleotides may be administered in various forms including one or more pharmaceutically acceptable salt forms. For example, in some embodiments, oligonucleotides are administered as dissolved oligonucleotides and/or salts thereof in suitable solutions, e.g., water or suitable buffer systems. In some embodiments, an amount of an oligonucleotide includes all forms of the oligonucleotide, and when expressed in weight (e.g., mg), such weight includes weights of all forms of the oligonucleotide but all converted to weight of the acid form (e.g., for WVE-003 using molecular weight 7257.94 free acid). Alternatively or additionally, amount of an oligonucleotide may be expressed in mole, which in some embodiments include moles of all form of the oligonucleotides (e.g., about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg of WVE-003 correspond to about 1.4, about 2.8, about 4.1, about 5.5, about 6.9, about 8.3, about 9.6, about 10.9, about 12.4, about 13.8, about 15.2, about 16.5, about 17.9, about 19.3, about 20.7, about 22.0 or about 23.1 umol WVE-003).

As described herein, multiple doses may be administered at various suitable frequencies, e.g., about every 3 weeks, about every 4 weeks, about every 8 weeks, about every 12 weeks, about once a month, about once bi-monthly or about once quarterly as described herein. In some embodiments, each does is independently equivalent to about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg WVE-003 free acid form. In some embodiments, a HTT oligonucleotide or composition is administered to a subject at a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg per month. In some embodiments, a HTT oligonucleotide or composition is administered to a subject at a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg every 8 weeks. In some embodiments, a HTT oligonucleotide or composition is administered to a subject at a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg every 12 weeks. In some embodiments, provided technologies provide long duration. In some embodiments, doses are administered every two months or less frequently. In some embodiments, doses are administered quarterly or less frequently.

In some embodiments, the present disclosure pertains to: a method of treatment and/or prevention of Huntington's disease in a subject (e.g., a patient such as a human patient) in need thereof; a method of allele-specific knockdown of a mutant Huntingtin transcript in a subject; and/or a method for reducing the severity of and/or delaying the onset of one or more symptoms of Huntington's disease, wherein the method comprises administering WVE-003 in multiple doses, each of which is independently equivalent to about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, or about 168 mg WVE-003 free acid form. In some embodiments, in the method of the present disclosure, the amount of each dose remains constant (e.g., each dose a patient receives is about 30 mg; or each dose a patient receives is about 60 mg; or each dose a patient receives is about 90 mg; or each dose a patient receives is about 120 mg); In some embodiments, the amount of each dose changes over time (e.g., a patient may be maintained for one or more doses at a particular dose, and later, due to efficacy and/or safety or other issues, the amount of each dose may be increased or decreased). In some embodiments, multiple doses are administered and each is independently about 30 mg WVE-003 free acid form. In some embodiments, multiple doses are administered approximately monthly or once approximately every 4 weeks. In some embodiments, the doses are administered approximately monthly or once approximately every 4 weeks, except for approximately two months or approximately eight weeks between the second and fourth doses. In some embodiments, multiple doses are administered once approximately every 8 weeks. For example, in some embodiments, a method comprises administering multiple doses once approximately every 8 weeks, each of which is independently equivalent to about 30 mg WVE-003 free acid form. In some embodiments, multiple doses are administered once approximately every 12 weeks. For example, in some embodiments, a method comprises administering multiple doses once approximately every 12 weeks, each of which is independently equivalent to about 30 mg WVE-003 free acid form. In some embodiments, each dose is administered as a pharmaceutical composition as described herein. In some embodiments, the doses are administered at least about 2 times. In some embodiments, the doses are administered at least about 3 times. In some embodiments, the doses are administered at least about 4 times. In some embodiments, interval between a dose and its immediate following dose is independently about 1, 2, 3, 4, 5, 6, 7, 8, 9, 10, 11, or 12 weeks, or about 1, 2, 3, 4, 5, 6, 7, 8, 9, 10, 11, 12 or more months. In some embodiments, interval between each dose and its immediate following dose is independently about 1, 2, 3, 4, 5, 6, 7, 8, 9, 10, 11, or 12 weeks, or about 1, 2, 3, 4, 5, 6, 7, 8, 9, 10, 11, 12 or more months. In some embodiments, an interval is about 1 week. In some embodiments, an interval is about 2 weeks. In some embodiments, an interval is about 3 weeks. In some embodiments, an interval is about 4 weeks. In some embodiments, an interval is about 5 weeks. In some embodiments, an interval is about 6 weeks. In some embodiments, an interval is about 7 weeks. In some embodiments, an interval is about 8 weeks. In some embodiments, an interval is about 9 weeks. In some embodiments, an interval is about 10 weeks. In some embodiments, an interval is about 9 weeks. In some embodiments, an interval is about 11 weeks. In some embodiments, an interval is about 9 weeks. In some embodiments, an interval is about 12 weeks. In some embodiments, an interval is about 1 month. In some embodiments, an interval is about 2 month. In some embodiments, an interval is about 3 or more months. In some embodiments, an interval is about 3, 4, 5, 6, 7, 8, or 9 months. In some embodiments, an interval is about 3 months. In some embodiments, an interval is about 6 months. In some embodiments, an interval is about 9 months. In some embodiments, each interval is independently about 1 or more weeks. In some embodiments, each interval is independently about 2 or more weeks. In some embodiments, each interval is independently about 3 or more weeks. In some embodiments, each interval is independently about 4 or more weeks. In some embodiments, each interval is independently about 5 or more weeks. In some embodiments, each interval is independently about 6 or more weeks. In some embodiments, each interval is independently about 7 or more weeks. In some embodiments, each interval is independently about 8 or more weeks. In some embodiments, each interval is independently about 9 or more weeks. In some embodiments, each interval is independently about 10 or more weeks. In some embodiments, each interval is independently about 11 or more weeks. In some embodiments, each interval is independently about 12 or more weeks. In some embodiments, each interval is independently about 1 or more months. In some embodiments, each interval is independently about 2 or more months. In some embodiments, each interval is independently about 3 or more months. In some embodiments, each interval is independently about 3, 4, 5, 6, 7, 8, or 9 months. In some embodiments, each interval is independently about 1 months. In some embodiments, each interval is independently about 2 months. In some embodiments, each interval is independently about 3 months. In some embodiments, each interval is independently about 6 months. In some embodiments, each interval is independently about 9 months. In some embodiments, all intervals are about the same. In some embodiments, one or more intervals are longer than others. In some embodiments, one or more intervals are shorter than others. In some embodiments, dose frequency for multiple or all doses is about 1, 2, 3, 4, 5, 6, 7, 8, 9, 10, 11 or 12 weeks, or about 1, 2, 3, 4, 5, 6, 7, 8, 9, 10, 11, 12 or more months. In some embodiments, dose frequency for multiple or all doses is about 1, 2, 3, 4, 5, 6, 7, 8, 9, 10, 11, 12 or more months. In some embodiments, one or more loading doses are administered. In some embodiments, no loading doses are administered.

In some embodiments, use of WVE-003 in various methods is tested in a clinical trial designated the SELECT-HD Phase 1b/2a trial. SELECT-HD is a 1b/2a multicenter, randomized, double-blind, placebo-controlled study to evaluate the safety, tolerability, PK, pharmacodynamics (PD), and clinical effects of WVE-003 in adult patients with early-manifest Huntington's disease (HD) who carry a targeted single nucleotide polymorphism (SNP) rs362273 (SNP3). Period 1 of this study evaluates single ascending doses (SAD) of WVE-003. Period 2 evaluates multiple ascending doses (MAD) of WVE-003. In some embodiments, a subject is with early manifest Huntington's disease. In some embodiments, a subject is 25 years old or older. In some embodiments, a subject is 60 years old or younger.

In some embodiments, an oligonucleotide, e.g., WVE-003, or a composition is administered intrathecally. In some embodiments, intrathecal administration is interspinal administration. In some embodiments, a method described herein comprises administering a HTT oligonucleotide, e.g., WVE-003, or a composition thereof intrathecally and in an amount described herein, e.g., equivalent to about 10, about 20, about 30, about 40, about 50, about 60, about 70, about 80, about 90, about 100, about 110, about 120, about 130, about 140, about 150, or about 168 mg oligonucleotide (e.g., WVE-003) free acid form. In some embodiments, a method described herein comprises administering a HTT oligonucleotide (e.g., WVE-003) or a composition thereof intrathecally and each independently in an amount described herein, e.g., equivalent to about 10, about 20, about 30, about 60, about 90, about 120, about 150, or about 168 mg oligonucleotide (e.g., WVE-003) free acid form with dose intervals described herein, e.g., monthly.

In some embodiments, a therapeutically effective amount of a HTT oligonucleotide, or a salt form thereof, or a HTT oligonucleotide composition elicits a desired biological response when administered as part of a therapeutic regimen. In some embodiments, a therapeutically effective amount of a HTT oligonucleotide, or a salt form thereof, or a HTT oligonucleotide composition is an amount (e.g., about 30, about 60, about 90, about 120, about 150, or about 168 mg free acid form) that is sufficient, when administered to a subject suffering from or susceptible to Huntington's disease, to treat, prevent, and/or delay the onset of Huntington's disease (e.g., at least one symptom of Huntington's disease).

In some embodiments, a method described herein comprises the step of administering a therapeutically effective amount of a HTT oligonucleotide or HTT oligonucleotide composition. In some embodiments, a HTT oligonucleotide or HTT oligonucleotide composition is or comprises WVE-003.

In some embodiments, a therapeutically effective amount of a HTT oligonucleotide, or a salt form thereof, or a HTT oligonucleotide composition is capable of mediating a clinically significant amount of allele-specific knockdown of a mutant HTT transcript. In some embodiments, a therapeutically effective amount of a HTT oligonucleotide, or a salt form thereof, or a HTT oligonucleotide composition does not significantly decrease the amount of wild-type Huntingtin transcript or a gene product thereof. In some embodiments, a therapeutically effective amount of a HTT oligonucleotide, or a salt form thereof, or a HTT oligonucleotide composition does not decrease the amount of wild-type Huntingtin transcript or a gene product thereof to a level associated with a clinically-manifested adverse event or side effect.

In some embodiments, a therapeutically effective amount of a HTT oligonucleotide, or a salt form thereof, or a HTT oligonucleotide composition is low enough to prevent or reduce the occurrence and/or reduce the severity of at least about one adverse event mediated by and/or associated with administration of the oligonucleotide or oligonucleotide composition to the subject. In some embodiments, a therapeutically effective amount of a HTT oligonucleotide, or a salt form thereof, or a HTT oligonucleotide composition is sufficient to mediate a clinically significant amount of allele-specific knockdown of a mutant HTT transcript in a subject, but is low enough to prevent or reduce the occurrence and/or reduce the severity of at least about one adverse event mediated by and/or associated with administration of the oligonucleotide or oligonucleotide composition to the subject.

In some embodiments, an adverse event is an adverse effect. In some embodiments, an adverse event is mild, moderate, severe, or serious. In some embodiments, an adverse event is: pyrexia, headache, vomiting, or tachycardia. In some embodiments, an adverse event is or is, is measured by, or is related to an increase in pro-inflammatory markers (e.g., C-reactive protein and complement), prolongation of a HTT, thrombocytopenia, liver enzyme changes (e.g., AST and ALT) or kidney biomarker changes (e.g., BUN, creatinine).

In some embodiments, a therapeutically effective amount of a HTT oligonucleotide, or a salt form thereof, or a HTT oligonucleotide composition is sufficient to achieve a clinically significant maximum plasma concentration of the HTT oligonucleotide or oligonucleotide composition. In some embodiments, a therapeutically effective amount of a HTT oligonucleotide, or a salt form thereof, or a HTT oligonucleotide composition is about 30 mg. In some embodiments, a therapeutically effective amount of a HTT oligonucleotide, or a salt form thereof, or a HTT oligonucleotide composition is about 60 mg. In some embodiments, a therapeutically effective amount of a HTT oligonucleotide, or a salt form thereof, or a HTT oligonucleotide composition is about 90 mg. In some embodiments, a therapeutically effective amount of a HTT oligonucleotide, or a salt form thereof, or a HTT oligonucleotide composition is about 120 mg. In some embodiments, a therapeutically effective amount of a HTT oligonucleotide, or a salt form thereof, or a HTT oligonucleotide composition is about 150 mg. In some embodiments, a therapeutically effective amount of a HTT oligonucleotide, or a salt form thereof, or a HTT oligonucleotide composition is about 168 mg. In some embodiments, a therapeutically effective amount of a HTT oligonucleotide, or a salt form thereof, or a HTT oligonucleotide composition is 30±25% mg. In some embodiments, a therapeutically effective amount of a HTT oligonucleotide, or a salt form thereof, or a HTT oligonucleotide composition is 60±25% mg. In some embodiments, a therapeutically effective amount of a HTT oligonucleotide, or a salt form thereof, or a HTT oligonucleotide composition is 90±25% mg. In some embodiments, a therapeutically effective amount of a HTT oligonucleotide, or a salt form thereof, or a HTT oligonucleotide composition is 120±25% mg. In some embodiments, a therapeutically effective amount of a HTT oligonucleotide, or a salt form thereof, or a HTT oligonucleotide composition is 150±25% mg. In some embodiments, a therapeutically effective amount of a HTT oligonucleotide, or a salt form thereof, or a HTT oligonucleotide composition is 168±25% mg. In some embodiments, a therapeutically effective amount of a HTT oligonucleotide, or a salt form thereof, or a HTT oligonucleotide composition is about 30 mg, about 60 mg, about 90 mg, about 120 mg, or about 150 mg (irrespective of body weight, as the total volume of the central nervous system is similar in most adult humans). In some embodiments, a therapeutically effective amount of a HTT oligonucleotide, or a salt form thereof, or a HTT oligonucleotide composition is about 30 mg. In some embodiments, a therapeutically effective amount of a HTT oligonucleotide, or a salt form thereof, or a HTT oligonucleotide composition is about 60 mg. In some embodiments, a therapeutically effective amount of a HTT oligonucleotide, or a salt form thereof, or a HTT oligonucleotide composition is about 90 mg. In some embodiments, a therapeutically effective amount of a HTT oligonucleotide, or a salt form thereof, or a HTT oligonucleotide composition is about 120 mg. In some embodiments, a therapeutically effective amount of a HTT oligonucleotide, or a salt form thereof, or a HTT oligonucleotide composition is about 150 mg. In some embodiments, a therapeutically effective amount of a HTT oligonucleotide, or a salt form thereof, or a HTT oligonucleotide composition is about 168 mg. In some embodiments, a HTT oligonucleotide is WVE-003.

In some embodiments, the present disclosure provides methods comprising administering to a subject a HTT oligonucleotide or HTT oligonucleotide composition in an amount of about 30, about 60, about 90, about 120, about 150, or about 168 mg equivalent to the free acid form of the oligonucleotide.

In some embodiments, the present disclosure provides methods comprising administering to a subject a HTT oligonucleotide or HTT oligonucleotide composition in an dose of about 30, about 60, about 90, about 120, about 150, or about 168 mg, wherein the oligonucleotide is WVE-003. In some embodiments, doses are administered approximately once a month, or approximately every four weeks. In some embodiments, doses are administered approximately once every two months, or approximately every eight weeks. In some embodiments, doses are administered approximately once every three months, or approximately every twelve weeks. In some embodiments, the time between the first and second doses is about 2 months or about 8 weeks, and each subsequent dose is administered approximately once a month, or approximately every four weeks. In some embodiments, the time between the first and second doses is about 3 months or about 12 weeks, and each subsequent dose is administered approximately once every two months, or approximately every 8 weeks. In some embodiments, each dose is administered approximately once every two months, or approximately every 8 weeks. In some embodiments, each dose is administered approximately once every two months, or approximately every 12 weeks. In some embodiments, the doses are administered for at least about 12 weeks. In some embodiments, the doses are administered for at least about 16 weeks. In some embodiments, the doses are administered for at least about 3 months. In some embodiments, the doses are administered for at least about 4 months. In some embodiments, a patient is maintained on the same dose or a constant dose (e.g., each dose a patient receives is 30 mg; or each dose a patient receives is 60 mg; or each dose a patient receives is 90 mg; or each dose a patient receives is 120 mg). In some embodiments, a patient receives multiple doses of different amounts. For example, a patient may receive one or more initial dose at a particular amount of HTT oligonucleotide or HTT oligonucleotide composition, and the subsequent doses the patient receives may be increased or decreased, due to efficacy, safety, or other issues.

Among other things, the present disclosure recognizes challenges of providing oligonucleotides with high efficacy and low toxicity, and methods of use thereof. In some embodiments, the present disclosure provides oligonucleotide, e.g., WVE-003, and compositions and methods with reduced toxicity. In some embodiments, provided technologies provide reduced toxicity levels when about the same or comparable amount of oligonucleotides are delivered, and/or when about the same or comparable levels of desired effects and/or effects are achieved. In some embodiments, at about the same or comparable or lower toxicity levels, more oligonucleotide, e.g., WVE-003, can be delivered, and/or higher levels of desired effects and/or effects are achieved. In some embodiments, a reference oligonucleotide is a non-selective oligonucleotide. In some embodiments, a reference oligonucleotide has a different structure compared to WVE-003. In some embodiments, a reference oligonucleotide has the same base sequence but different structure compared to WVE-003. In some embodiments, a reference oligonucleotide comprises different modifications and/or patterns thereof. In some embodiments, a reference oligonucleotide comprises different internucleotidic linkages and/or patterns thereof. In some embodiments, a reference oligonucleotide comprises different linkage stereochemistry or patterns thereof. In some embodiments, a reference oligonucleotide is provided as a stereorandom composition. In some embodiments, a reference oligonucleotide is provided as a chirally controlled oligonucleotide composition. In some embodiments, a reference oligonucleotide is of lower purity. In some embodiments, a reference oligonucleotide is of lower stereopurity. In some embodiments, a reference oligonucleotide is WV-1092. In some embodiments, a reference oligonucleotide is WV-2603.

In some embodiments, the present disclosure provides oligonucleotide compositions and methods with reduced immune responses. In some embodiments, the present disclosure recognizes that various toxicities induced by oligonucleotides may be related to cytokine and/or complement activation.

In some embodiments, a HTT oligonucleotide composition (e.g., WVE-003) is chirally controlled (e.g., stereopure). Among other things, a stereorandom HTT oligonucleotide preparations contain a plurality of distinct chemical entities that differ from one another, e.g., in the stereochemical structure of individual backbone chiral centers (e.g., phosphorothioates) within the HTT oligonucleotide chain. Without control of stereochemistry of backbone chiral centers, stereorandom HTT oligonucleotide preparations provide uncontrolled (or stereorandom) compositions comprising undetermined levels of HTT oligonucleotide stereoisomers. Even though these stereoisomers may have the same base sequence and/or chemical modifications, they are different chemical entities at least about due to their different backbone stereochemistry, and they can have, as demonstrated herein, different properties, e.g., activities, toxicities, distribution etc. Among other things, the present disclosure provides chirally controlled compositions that are or contain particular stereoisomers of HTT oligonucleotides of interest; in contrast to chirally uncontrolled compositions, chirally controlled compositions comprise controlled levels of particular stereoisomers of HTT oligonucleotides, or in a chirally controlled composition a controlled level of all oligonucleotides of the composition, or a controlled level of all oligonucleotides of the composition that share a particular base sequence (e.g., that of WVE-003) share a common pattern of linkage phosphorus stereochemistry configuration (e.g., that of WVE-003). In some embodiments, a controlled level of all oligonucleotides that share the same constitution as a form of WVE-003 are WVE-003. In some embodiments, diastereopurity of WVE-003 is about or above a controlled level.

In some embodiments, a controlled level is at least about 10%. In some embodiments, a controlled level is at least about 20%. In some embodiments, a controlled level is at least about 25%. In some embodiments, a controlled level is at least about 30%. In some embodiments, a controlled level is at least about 40%. In some embodiments, a controlled level is at least about 50%. In some embodiments, a controlled level is at least about 60%. In some embodiments, a controlled level is at least about 70%. In some embodiments, a controlled level is at least about 75%. In some embodiments, a controlled level is at least about 80%. In some embodiments, a controlled level is at least about 85%. In some embodiments, a controlled level is at least about 90%. In some embodiments, a controlled level is at least about 95%. In some embodiments, a controlled level is at least about 96%. In some embodiments, a controlled level is at least about 97%. In some embodiments, a controlled level is at least about 98%. In some embodiments, a controlled level is at least about 99%.

In some embodiments, a therapeutically effective amount of a HTT oligonucleotide, a HTT oligonucleotide composition, WVE-003, is about 30, about 60, about 90, about 120, about 150 or about 168 mg.

In some embodiments, a HTT oligonucleotide, or a salt form thereof, or a HTT oligonucleotide composition is or comprises WVE-003, which is a chirally controlled HTT oligonucleotide composition capable of mediating an allele-specific decrease in the level, expression and/or activity of a mHTT transcript.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of 10 to about 168 mg, such that disease progression in the subject is delayed, and/or the onset of Huntington's disease is delayed, and/or the severity of a symptom of Huntington's disease is reduced.

In some embodiments, a HTT gene in a human subject is: (A) heterozygous at the CAG repeat (wherein one allele comprises a deleterious CAG repeat expansion and the other allele does not), and (B) heterozygous at a SNP targeted by a HTT oligonucleotide, wherein the HTT oligonucleotide is capable of differentiating between two alleles of the same SNP (e.g., due to a difference in the sequence of the SNP alleles); and (C) the deleterious CAG repeat is on the same chromosome as the SNP allele targeted by the HTT oligonucleotide; and such an arrangement is referenced as the CAG repeat expansion and the mutant SNP allele being in phase, and the HTT gene or a mutation thereof can be described as amenable to allele-specific knockdown.

In some embodiments, a mutant HTT gene comprising a mutation that is amenable to an allele-specific knockdown of the mutant HTT gene is: a mutant HTT gene comprising a deleterious mutation (e.g., a CAG repeat expansion), wherein the deleterious mutation is on the same chromosome as (e.g., in phase with, or in the same phase as) a particular allele of a SNP which is targeted by a particular HTT oligonucleotide (e.g., WVE-003), wherein targeting by the oligonucleotide of the SNP allele also targets the deleterious mutation.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 60 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 90 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 120 mg, such that disease progression in the subject is delayed, and/or the onset of Huntington's disease is delayed, and/or the severity of a symptom of Huntington's disease is reduced.

In some embodiments, a subject is determined to express a mtHTT transcript that is full complementary to the sequence an administered oligonucleotide, including at a SNP site (e.g., rs362273). In some embodiments, a subject is determined to have a mtHTT allele that comprises the same sequence (as appreciated by those skilled in the art U and T may be properly considered the same) or is fully complementary to the sequence of an administered oligonucleotide, including at a SNP site (e.g., rs362273). In some embodiments, a subject is homozygous with respect to a SNP site and/or wild-type or mutant HTT. In some embodiments, a subject is heterozygous with respect to a SNP site and/or wild-type or mutant HTT. In some embodiments, a subject expresses a mtHTT transcript that is fully complementary to an administered oligonucleotide, and a wtHTT transcript that is not fully complementary to an administered oligonucleotide. In some embodiments, a subject has that a mtHTT allele that comprises the same sequence (as appreciated by those skilled in the art U and T may be properly considered the same) or is fully complementary to the sequence of an administered oligonucleotide, and a wtHTT allele that does not comprise a sequence that is the same (as appreciated by those skilled in the art U and T may be properly considered the same) or is fully complementary to the sequence of an administered oligonucleotide. In some embodiments, levels of transcripts, proteins and/or activities of a mtHTT allele is reduced more than those of a wtHTT. a mtHTT allele that is fully complementary to an administered oligonucleotide, and a wtHTT transcript that is not fully complementary to an administered oligonucleotide. Various technologies are available and can be utilized in accordance with the present disclosure to detect and/or determine if a particular isoform of a SNP is on the same allele or transcript.

In some embodiments, the present disclosure provides for a method for determining the suitability of treatment of a subject for administration of WVE-003 (or a salt form thereof), said method comprising the steps of:
i) determining the presence of mutant allele in a patient with the targeted single nucleotide polymorphism (SNP) rs362273 (SNP3) versus the wild type allele;
ii) comparing the level of mutant allele in a patient with the targeted single nucleotide polymorphism (SNP) rs362273 (SNP3) of step i) with one or more reference samples or reference values;
iii) determining whether the subject is likely to be, or is suitable for, treatment with WVE-003, wherein the subject is suffering from or at risk of developing an neurodegenerative disorder, such as Huntington's disease; and
iv) administering an effective amount of WVE-003 to the subject.

In some embodiments, the present disclosure provides for the use of a phasing assay in the detection of the presence of a mutant allele in a patient with the targeted single nucleotide polymorphism (SNP) rs362273 (SNP3) versus the wild type allele to determine suitability of treatment with WVE-003.

Various techniques can be used to determine if a particular SNP allele is on the same chromosome as a disease-associated sequence, e.g., CAG repeat expansion for HTT. Typically, if the SNP allele and the CAG repeat expansion are on the same chromosome, an HTT oligonucleotide that targets that SNP allele can also “target” the disease-associated CAG repeat expansion, thereby allowing a decrease in the expression, level and/or activity of the HTT allele with the disease-associated mutation. In such a way, for example, an HTT oligonucleotide can be used in a treatment for an HTT-related disorder such as Huntington's disease. An HTT oligonucleotide targeting a SNP can thus preferentially decrease the expression, level and/or activity of a mutant allele of HTT compared to the wild-type allele.

Humans, among other living things, are diploid, and determining the linkage of alleles of genetic loci on the same or different chromosomes is desirable for phasing techniques. The sequences on corresponding chromosomes are known as haplotypes. The process of determining which alleles are on which chromosomes is known as phasing, haplotype phasing or haplotyping. Phasing information is useful in patient stratification, forensics and various other applications in the treatment of HTT-related diseases and disorders such as Huntington's diseases. For additional general information about phasing, see, for example: Twehey et al. 2011 Nat. Rev. Genet. 12: 215-223; and Glusman et al. 2014 Genome Med. 6:73.

Phasing data can be important in allele-specific therapies for diseases such as Huntington's disease. In some diseases, a genetic lesion such as a deleterious repeat, deletion, insertion, inversion or other mutation has been identified, such as an expanded CAG repeat expansion in mutant (and disease-associated) HTT alleles. In some patients, one allele of a gene such as HTT can comprise a disease-associated mutation at a genetic locus, while the other allele is normal, wild-type or otherwise not or less disease-associated. In some embodiments, an allele-specific therapy can target an allele of HTT comprising a disease-associated mutation, but not the corresponding wild-type allele. In some embodiments, an allele-specific therapy can target an HTT allele comprising a disease-associated mutation at a particular locus, such as a CAG repeat expansion (or expanded CAG tract), but not by directly targeting the locus, but rather by targeting a different locus on the mutant allele. As a non-limiting example, an allele-specific therapy can target an allele comprising a disease-associated mutation at a locus by targeting a different locus in the same allele, such as a SNP (single nucleotide polymorphism) in the same gene.

As a non-limiting example, some disease-associated genetic lesions may be difficult to target or otherwise not readily amenable to targeting. As a non-limiting example, some genes such as mutant HTT comprise repeats (e.g., trinucleotide or tetranucleotide repeats); in some cases, such as Huntington's disease, a small number of repeats is not disease-associated, but an abnormally large number of repeats, or a repeat expansion, is disease-associated. Because the repeats exist on both the wild-type and mutant alleles, it may be difficult to target the disease-associated repeats directly. However, if a particular SNP variant exists on the same allele as the disease-associated repeat expansion but not on the wild-type allele, that SNP variant can be used to target an allele-specific therapy which targets the mutant allele but not the wild-type allele.

As a non-limiting example, phasing data for an individual indicates if a particular SNP is in phase (e.g., on the same chromosome or transcript) as the lesion and thus that SNP can be targeted with a therapeutic nucleic acid. The therapeutic can then target the mutant gene, while not targeting the wild-type allele. Obtaining the phasing data to target only the mutant allele can be especially useful if expression of the wild-type allele is essential.

As another non-limiting example, phasing information is useful if it is known that an individual has both a wild-type and a mutant allele of each of two genetic loci on the same gene. Phasing information will reveal if both copies of the gene each have one mutant allele, or if one copy of the gene has two mutations, while the other is wild-type at both alleles.

In some embodiments, the present disclosure presents, inter alia, various methods for phasing genetic loci on a nucleic acid template. As non-limiting examples, the present disclosure presents methods for phasing a genetic locus such as a genetic lesion (such as an inversion, fusion, deletion, insertion or other mutation) and another genetic locus (such as a SNP) on a chromosome; the two genetic loci can be in the same gene, or in different genes.

In a non-limiting example, an example patient may have Huntington's Disease, which is linked to a mutation in the Huntingtin gene (HTT) comprising an excessive number of repeats (e.g., a repeat expansion) of the sequence CAG. In some embodiments, the patient may be under consideration for treatment with an allele-specific therapeutic (e.g., an antisense oligonucleotide or RNAi agent) which recognizes a particular allelic variant of a genetic locus in the HTT gene (which is outside the repeat expansion), as a non-limiting example, a SNP. If phasing reveals that the same chromosome of the patient comprises both the repeat expansion and the particular allelic variant of a genetic locus (e.g., a SNP) recognized by the allele-specific therapeutic, then the patient is eligible for treatment with the allele-specific therapeutic.

Various methods for phasing are known in the art, including but not limited to those described in: WO2018/022473; and Berger et al. 2015 Res. Comp. Mol. Biol. 9029: 28-29; Castel et al. 2015 Genome Biol. 16: 195; Castel et al. 2016 phASER: Long range phasing and haplotypic expression from RNA sequencing, doi: http://dx.doi.org/10.1101/039529; Delaneau et al. 2012 Nat. Methods 9: 179-181; Garg et al. 2016 Read-Based Phasing of Related Individuals; Hickey et al. 2011 Genet. Select. Evol. 43:12; Kuleshov et al. 2014 Nat. Biotech. 32: 261-266; Laver et al. 2016 Nature Scientific Reports|6:21746|DOI: 10.1038/srep21746; O'Connell et al. 2014 PLoS ONE 10: e1004234; Regan et al. 2015 PloS ONE 10: e0118270; Roach et al. 2011 Am. J. Hum. Genet. 89: 382-397; and Yang et al. 2013 Bioinformatics 29: 2245-2252. In some embodiments, sequencing, particularly sequencing that can produce long single reads, can be utilized for phasing.

One example of a phasing assay that can be suitably employed for detection of mutant allele in a patient is allele-specific PCR or allele-specific long-range PCR, such assay can be performed which employs allele-specific primers to detect mutations in nucleic acid sequences in the presence of wild-type variants of the sequences. Allele-specific PCR is a technique in which the variant of the nucleic acid sequence present in the PCR reaction mixture is selectively amplified and detected. Allele-specific PCR employs at least one “allele-specific primer.” The term “allele-specific” primer generally refers to a primer whose extension occurs in a PCR reaction only when a specific variant of a nucleic acid sequence is present in the reaction mixture. In other words, allele-specific primers are designed in such a way that they discriminate between variants of nucleic acids and selectively multiply nucleic acid templates that include a variant to be detected.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 150 mg, such that disease progression in the subject is delayed, and/or the onset of Huntington's disease is delayed, and/or the severity of a symptom of Huntington's disease is reduced.

In some embodiments of the methods described herein, nucleic acid sequences are detected by suitable methods, such as quantitative amplification and/or nucleic acid sequencing. Methods of quantitative amplification are disclosed in, e.g., U.S. Pat. Nos. 5,210,015; 5,804,375; 6,127,155; 6,180,349; 6,033,854; and 5,972,602, as well as in, e.g., Holland et al, Proc. Natl. Acad. Sci. 88:7276-7280 (1991), Gibson et al, Genome Research 6:995-1001 (1996); DeGraves, et al, Biotechniques 34(1):106-10, 112-5 (2003); Deiman B, et al, Mol Biotechnol. 20(2):163-79 (2002). Amplifications may be monitored in “real time.” Though standard Sanger dideoxy or other older nucleotide sequencing methods can be used, sequencing can be particularly effective when high throughput sequencing is used, e.g., “next generation sequencing” methods such as HiSeq™, MiSeq™, or Genome Analyzer (each available from Illumina), SOLiD or Ion Torrent™ (each available from Life Technologies) and 454™ sequencing (from Roche Diagnostics). For example, in high-throughput sequencing, parallel sequencing reactions using multiple templates and multiple primers allows rapid sequencing of genomes or large portions of genomes. See, e.g., WO 03/004690, WO 03/054142, WO 2004/069849, WO 2004/070005, WO 2004/070007, WO 2005/003375, WO 00/06770, WO 00/27521, WO 00/58507, WO 01/23610, WO 01/57248, WO 01/57249, WO 02/061127, WO 03/016565, WO 03/048387, WO 2004/018497, WO 2004/018493, WO 2004/050915, WO 2004/076692, WO 2005/021786, WO 2005/047301, WO 2005/065814, WO 2005/068656, WO 2005/068089, WO 2005/078130, and Seo, et al, Proc. Natl Acad. Sci. USA (2004) 101: 5488-5493. In some embodiments, the amplicons are sequenced by one of the methods selected from a base-incorporation method, e.g., a pyrosequencing method (U.S. Pat. Nos. 6,274,320, 6,258,568 and 6,210,891); a hydrogen ion detection method (ISFET) (e.g., U.S. Pat. No. 8,262,900), or a dye-terminator detection method (U.S. Pat. Nos. 7,835,871, 8,244,479, 8,315,817 and 8,412,467.) Deep sequencing technology and instruments (e.g., technology and instrument capable of digital sequence readout) may also be employed. Without limitation, the examples of instruments include GS family of instruments (454 Life Sciences, Branford, Conn.); ION PROTON* and PGM™ (Life Technologies, Grand Island, N.Y.); HISEQ* and MISEQ* (Illumina, San Diego, Cal.) or any improvements and modifications of thereof. In some embodiments, a sequencing technology is or comprises long-read sequencing. In some embodiments, a long-read sequencing covers two or more sequence elements, for example, in some embodiments, one is a SNP and the other is a mutation (e.g., a point mutation, a CAG repeat, etc.).

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 168 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 60 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 90 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 120 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 150 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 168 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 60 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 90 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 120 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 150 mg, such that disease progression in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 168 mg, such that disease progression in the subject is delayed, and/or the onset of Huntington's disease is delayed, and/or the severity of a symptom of Huntington's disease is reduced.

In some embodiments, the present disclosure pertains to: A method delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 30 mg.

In some embodiments, the present disclosure pertains to: A method delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 at a dose of about 60 mg.

In some embodiments, the present disclosure pertains to: A method delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 90 mg.

In some embodiments, the present disclosure pertains to: A method delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 120 mg.

In some embodiments, the present disclosure pertains to: A method delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 150 mg.

In some embodiments, the present disclosure pertains to: A method delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 168 mg.

As described herein, an amount of WVE-003 typically include all forms of WVE-003 being administered but all converted to the amount of the acid form.

In some embodiments, the present disclosure pertains to: The method of any of the previous embodiments, wherein the subject is administered a steroid at least about one month prior to the first dose of WVE-003.

In some embodiments, the present disclosure pertains to: The method of any of the previous embodiments, wherein the subject is administered WVE-003 approximately monthly for at least about 4 months.

In some embodiments, the present disclosure pertains to: The method of any of the previous embodiments, wherein the subject is administered WVE-003 approximately monthly for at least about 8 months.

In some embodiments, the present disclosure pertains to: The method of any of the previous embodiments, wherein the subject is administered the oligonucleotide approximately monthly for at least about 12 months.

In some embodiments, the present disclosure pertains to: The method of any of the previous embodiments, wherein the subject is administered the oligonucleotide approximately monthly for at least about 16 months.

In some embodiments, the present disclosure pertains to: The method of any of the previous embodiments, wherein the subject is administered the oligonucleotide approximately monthly for at least about 48 months.

In some embodiments, the present disclosure pertains to: The method of any of the previous embodiments, wherein the oligonucleotide is delivered intrathecally.

In some embodiments, the present disclosure pertains to: The method of any of the previous embodiments, wherein the method further comprises the step of confirming that the subject has a mutation in the HTT gene that is amenable to an allele-specific knockdown of the mutant HTT gene or a gene product thereof transcript.

In some embodiments, the present disclosure pertains to: The method of any of the previous embodiments, wherein the oligonucleotide is formulated as a liquid formulation.

In some embodiments, the method includes the step of lyophilization (e.g., freeze-drying or freeze-drying under a vacuum).

In some embodiments, the present disclosure pertains to: The method of any of the previous embodiments, wherein the oligonucleotide is formulated as a liquid formulation reconstituted from a lyophilized (e.g., freeze-dried) preparation of the oligonucleotide.

In some embodiments, the present disclosure pertains to: a lyophilized (e.g., freeze-dried) preparation of WVE-003.

In some embodiments, the present disclosure pertains to: The method of any of the previous embodiments, wherein the oligonucleotide is formulated as a liquid formulation, wherein the liquid formulation comprises the oligonucleotide, sodium chloride and water.

In some embodiments, the present disclosure pertains to: The method of any of the previous embodiments, wherein the oligonucleotide is formulated as a liquid formulation, wherein the liquid formulation is reconstituted from a lyophilized preparation with a solution of sodium chloride.

In some embodiments, the present disclosure pertains to: The method of any of the previous embodiments, wherein the oligonucleotide is formulated as a liquid formulation, wherein the liquid formulation is reconstituted from a lyophilized preparation with a solution of 0.9% sodium chloride.

In some embodiments, the present disclosure pertains to: The method of any of the previous embodiments, wherein the oligonucleotide is formulated as a liquid formulation, wherein the liquid formulation is reconstituted from a lyophilized preparation with a solution of sterile sodium chloride.

In some embodiments, the present disclosure pertains to: The method of any of the previous embodiments, wherein the oligonucleotide is formulated as a liquid formulation, wherein the liquid formulation is reconstituted from a lyophilized preparation with 0.9% sterile sodium chloride.

In some embodiments, a lyophilized preparation of WVE-003 is a dry (or dried) solid.

In some embodiments, a lyophilized preparation of WVE-003 is a dry powder.

In some embodiments, a lyophilized preparation of WVE-003 is a dry powder prepared by lyophilization of a liquid formulation of WVE-003 in water.

In some embodiments, a lyophilized preparation of WVE-003 is a dry powder in a single-use clear glass vial.

In some embodiments, a lyophilized preparation of WVE-003 is about 20 mg of a dry powder in a vial.

In some embodiments, a lyophilized preparation of WVE-003 is a dry powder in a 10 mL vial.

In some embodiments, a lyophilized preparation of WVE-003 is about 20 mg of a dry powder in a 10 mL vial.

In some embodiments, the subject is administered a steroid prior to the first dose of WVE-003.

In some embodiments, the subject is administered a steroid at least about one month prior to the first dose of WVE-003.

In some embodiments, the subject is administered a dose of WVE-003 approximately once a month, or approximately every four weeks.

In some embodiments, the subject is administered a dose of WVE-003 approximately once every two months or approximately every eight weeks.

In some embodiments, the subject is administered a dose of WVE-003 approximately once a month, or approximately every four weeks, except that the time between the first and second doses is about 2 months or about 8 weeks, and each subsequent dose is administered approximately once a month, or approximately every four weeks.

In some embodiments, the subject is administered a dose of WVE-003 approximately once every two months, or approximately every 8 weeks, except that the time between the first and second doses is about 3 months or about 12 weeks, and each subsequent dose is administered approximately once every two months, or approximately every 8 weeks.

In some embodiments, the subject is administered WVE-003 approximately every 8 weeks for at least about 16 weeks.

In some embodiments, the subject is administered WVE-003 approximately every 12 weeks for at least about 12 weeks.

In some embodiments, the subject is administered WVE-003 approximately monthly for at least about 4 months.

In some embodiments, the subject is administered WVE-003 approximately monthly for at least about 8 months.

In some embodiments, the subject is administered WVE-003 approximately monthly for at least about 12 months.

In some embodiments, the subject is administered WVE-003 approximately monthly for at least about 16 months.

In some embodiments, the subject is administered WVE-003 approximately monthly for at least about 48 months.

In some embodiments, the subject is administered a steroid prior to the first dose of a HTT oligonucleotide or a HTT oligonucleotide composition.

In some embodiments, the subject is administered a steroid at least about one month prior to the first dose of a HTT oligonucleotide or a HTT oligonucleotide composition.

In some embodiments, the subject is administered hydrocortisone and/or acetaminophen within 24 hours of administration of a HTT oligonucleotide or a HTT oligonucleotide composition.

In some embodiments, the subject is administered a HTT oligonucleotide or a HTT oligonucleotide composition approximately monthly for at least about 8 months.

In some embodiments, the subject is administered a HTT oligonucleotide or a HTT oligonucleotide composition approximately monthly for at least about 12 months.

In some embodiments, the subject is administered a HTT oligonucleotide or a HTT oligonucleotide composition approximately monthly for at least about 16 months.

In some embodiments, the subject is administered a HTT oligonucleotide or a HTT oligonucleotide composition approximately monthly for at least about 48 months.

In some embodiments, the method further comprises the step of confirming that the subject has a mutation in the HTT gene that is amenable to an allele-specific decrease in the level, expression and/or activity of a mHTT transcript.

In some embodiments, the method further comprises the step of confirming that the subject has a mutation in the HTT gene that is amenable to allele-specific knockdown of a mutant HTT transcript.

In some embodiments, the present disclosure provides a pharmaceutical composition comprising a HTT oligonucleotide or a HTT oligonucleotide composition of the present disclosure (e.g., WVE-003) and a pharmaceutically acceptable carrier.

In some embodiments, the present disclosure provides a pharmaceutical composition comprising a HTT oligonucleotide or a HTT oligonucleotide composition of the present disclosure (e.g., WVE-003), which can be utilized in pharmaceutical compositions by combining such oligomeric compounds with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in certain embodiments, employed in the methods described herein is a pharmaceutical composition comprising a HTT oligonucleotide or a HTT oligonucleotide composition of the present disclosure (e.g., WVE-003), and a pharmaceutically acceptable diluent. In certain embodiments, the pharmaceutically acceptable diluent is PBS. In certain embodiments, the pharmaceutically acceptable diluent is artificial CSF (aCSF).

In certain embodiments, pharmaceutical compositions are administered both directly to the CSF (e.g., IT and/or ICV injection and/or infusion) and systemically.

In some embodiments, the present disclosure provides a method for allele-specific knockdown of a target HTT transcript, comprising the step of administering a HTT oligonucleotide composition of the present disclosure. In some embodiments, the present disclosure provides a method for reducing level of a HTT transcript or a product thereof, comprising the step of administering a HTT oligonucleotide composition of the present disclosure. A method for treating Huntington's disease, comprising the step of administering to a subject susceptible thereto or suffering therefrom a composition described in the present disclosure.

In some embodiments, the present disclosure provides a method for treating Huntington's disease, comprising the step of administering to a subject susceptible thereto or suffering therefrom a composition comprising any HTT oligonucleotide disclosed herein.

In some embodiments, the present disclosure provides a method for treating Huntington's disease, comprising (a) administering to a subject susceptible thereto or suffering therefrom a composition comprising any HTT oligonucleotide disclosed herein, and (b) administering to the subject additional treatment which is capable of preventing, treating, ameliorating or slowing the progress of Huntington's disease. In some embodiments, the present disclosure provides a method comprising administering to a subject WVE-003, wherein the subject is determined to have a genetic sequence that is the same or fully complementary to the base sequence of WVE-003. In some embodiments, the present disclosure provides a method comprising administering to a subject WVE-003, wherein the subject is determined to have a genetic sequence that comprises a sequence that is the same or fully complementary to the base sequence of WVE-003 and a sequence that is or encodes an expanded CAG repeats. In some embodiments, the present disclosure provides a method comprising administering to a subject WVE-003, wherein the subject is determined to have a genetic sequence that encodes a transcript that comprises an expanded CAG repeat in HTT and is fully complementary to the base sequence of WVE-003. In some embodiments, the present disclosure provides a method comprising administering to a subject WVE-003, wherein the subject is determined to express a HTT transcript that comprises an expanded CAG repeat and is fully complementary to the base sequence of WVE-003.

The present disclosure also provides various formulations of a HTT oligonucleotide or HTT oligonucleotide composition, any of which can be used in any method described herein.

In some embodiments, in any method described herein, the level, expression and/or activity of mutant HTT (e.g., a transcript and/or a gene product thereof) is reduced by at least about 5%.

In some embodiments, in any method described herein, the level, expression and/or activity of mutant HTT (e.g., a transcript and/or a gene product thereof) is reduced by at least about 10%.

### BRIEF DESCRIPTION OF THE DRAWINGS

FIG. 1. An example flow diagram for WVE-003 drug substance manufacturing process including in-process controls.

FIG. 2. An example manufacturing process flow diagram for a WVE-003 drug product. Abbreviations: DS: drug substance; IPC: in process control; IPT: in process test.

FIG. 3. Example schematic of a study design. Abbreviations: DEC=Dose Escalation Committee; MAD: multiple ascending dose; N: number of patients; PxCx: Period x Cohort x; SAD: single ascending dose. ^{a}If P1C2 is not conducted, then 6 new patients (2:1 active:placebo) are enrolled in P2C1.

FIG. 4. Blinded CSF PK data comparing dose of WVE-003 to doses of WVE-120101 (WV-1092) and WVE-120102 (WV-2603).

FIG. 5. Provided technologies can reduce mHTT protein levels without reducing wtHTT protein levels in human subjects. Shown are certain percentage changes from baseline in CSF mHTT and wtHTT proteins through day 85 in certain subjects to whom placebo (aCSF), WVE-003 30 mg or WVE-003 60 mg single doses were administered, respectively. As demonstrated, in several human subjects 30 mg and 60 mg single doses of WVE-003 resulted in durable mHTT reductions (A) without reducing wtHTT protein levels (B). For pooled single 30 mg and 60 mg doses, a 22% mean reduction (30% median reduction) of mHTT was observed (C) while no reduction of wtHTT was observed (D). Mixed model for repeated measures was used displaying geometric mean ratio to baseline. For each time point, two bars (representing 95% CI) are presented: solid for WVE-003 (30 mg or 60 mg) and dashed for placebo.

FIG. 6. Example schematic of a study design.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; and (v) where ranges are provided, endpoints are included.

Unless otherwise specified, description of oligonucleotides and elements thereof (e.g., base sequence, sugar modifications, internucleotidic linkages, linkage phosphorus stereochemistry, etc.) is from 5′ to 3′. Unless otherwise specified, oligonucleotides described herein may be provided and/or utilized in salt forms, particularly pharmaceutically acceptable salt forms. As those skilled in the art will appreciate after reading the present disclosure, in some embodiments, oligonucleotides may be provided as salts, but are not limited to, e.g., sodium or potassium salts. As those skilled in the art will appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition)), and will understand that, so long as their acid forms (e.g., replacing all cations, if any, with H) are of the same constitution and/or structure, such individual oligonucleotides may properly be considered to be of the same constitution and/or structure.

Approximately: As used herein, the terms “about” and “approximately” may be understood to permit standard variation as would be understood by those of ordinary skill in the art. In some embodiments, the terms “approximately” or “about” in reference to a number are generally taken to include numbers that fall within a range of 5%, 10%, 15%, 20%, 25%, or 30%, in either direction (greater than or less than) of the number unless otherwise stated or otherwise evident from the context.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Pharmaceutical composition: As used herein, the term “pharmaceutical composition” refers to an active agent, formulated together with one or more pharmaceutically acceptable carriers. In some embodiments, active agent is present in a unit dose amount appropriate for administration in a therapeutic regimen that shows a statistically significant probability of achieving a controlled therapeutic effect when administered to a relevant population.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, a provided compound comprises more than one acid groups, for example, a provided oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), each acidic group having sufficient acidity independently exists as its salt form (e.g., in an oligonucleotide comprising natural phosphate linkages and phosphorothioate internucleotidic linkages, each of the natural phosphate linkages and phosphorothioate internucleotidic linkages independently exists as its salt form). In some embodiments, a pharmaceutically acceptable salt of an oligonucleotide is a sodium salt of a provided oligonucleotide. In some embodiments, a pharmaceutically acceptable salt of an oligonucleotide is a sodium salt of a provided oligonucleotide, wherein each acidic linkage, e.g., each natural phosphate linkage and phosphorothioate internucleotidic linkage, exists as a sodium salt form (all sodium salt).

Subject: As used herein, the term “subject” or “test subject” refers to any organism to which a provided compound or composition is administered in accordance with the present disclosure e.g., for experimental, diagnostic, prophylactic, and/or therapeutic purposes. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: In some embodiments, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount. In some embodiments, a single dose is an infusion, which may take up to one or more hours.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Chirally controlled oligonucleotide composition: The terms “chirally controlled (stereocontrolled or stereodefined) oligonucleotide composition”, “chirally controlled (stereocontrolled or stereodefined) nucleic acid composition”, and the like, as used herein, refers to a composition that comprises a plurality of oligonucleotides (or nucleic acids, chirally controlled oligonucleotides or chirally controlled nucleic acids) which share 1) a common base sequence, 2) a common pattern of backbone linkages; 3) a common pattern of backbone chiral centers, and 4) a common pattern of backbone phosphorus modifications (oligonucleotides of a particular type), wherein the plurality of oligonucleotides (or nucleic acids) share the same stereochemistry at one or more chiral internucleotidic linkages (chirally controlled internucleotidic linkages, whose chiral linkage phosphorus is Rp or Sp, not a random Rp and Sp mixture as non-chirally controlled internucleotidic linkages). Level of the plurality of oligonucleotides (or nucleic acids) in a chirally controlled oligonucleotide composition is non-random (pre-determined, controlled). Chirally controlled oligonucleotide compositions are typically prepared through chirally controlled oligonucleotide preparation to stereoselectively form one or more chiral internucleotidic linkages (e.g., using chiral auxiliaries as exemplified in the present disclosure, compared to non-chirally controlled (stereorandom, non-stereoselective, racemic) oligonucleotide synthesis such as traditional phosphoramidite-based oligonucleotide synthesis using no chiral auxiliaries or chiral catalysts to purposefully control stereoselectivity). A chirally controlled oligonucleotide composition is enriched, relative to a substantially racemic preparation of oligonucleotides having the common base sequence, the common pattern of backbone linkages, and the common pattern of backbone phosphorus modifications, for oligonucleotides of the plurality. In some embodiments, a chirally controlled oligonucleotide composition comprises a plurality of oligonucleotides of a particular oligonucleotide type defined by: 1) base sequence; 2) pattern of backbone linkages: 3) pattern of backbone chiral centers; and 4) pattern of backbone phosphorus modifications, wherein it is enriched, relative to a substantially racemic preparation of oligonucleotides having the same base sequence, pattern of backbone linkages, and pattern of backbone phosphorus modifications, for oligonucleotides of the particular oligonucleotide type. As one having ordinary skill in the art readily appreciates, such enrichment can be characterized in that compared to a substantially racemic preparation, at each chirally controlled internucleotidic linkage, a higher level of the linkage phosphorus has the desired configuration. In some embodiments, each chirally controlled internucleotidic linkage independently has a diastereopurity of at least 80%, 85%, 90%, 91%, 92%, 93%, 94%, 95%, 96%, 97%, 98%, or 99% with respect to its chiral linkage phosphorus. In some embodiments, each independently has a diastereopurity of at least 90%. In some embodiments, each independently has a diastereopurity of at least 95%. In some embodiments, each independently has a diastereopurity of at least 97%. In some embodiments, each independently has a diastereopurity of at least 98%. In some embodiments, oligonucleotides of a plurality have the same constitution. In some embodiments, oligonucleotides of a plurality have the same constitution and stereochemistry, and are structurally identical.

In some embodiments, the plurality of oligonucleotides in a chirally controlled oligonucleotide composition share the same base sequence, the same, if any, nucleobase, sugar, and internucleotidic linkage modifications, and the same stereochemistry (Rp or Sp) independently at linkage phosphorus chiral centers of one or more chirally controlled internucleotidic linkages, though stereochemistry of certain linkage phosphorus chiral centers may differ. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence are oligonucleotides of the plurality. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, about 0.1%-100%, (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications (e.g., of a plurality of oligonucleotide or an oligonucleotide type), or of all oligonucleotides in a composition that share a common base sequence, a common patter of base modifications, a common pattern of sugar modifications, a common pattern of internucleotidic linkage types, and/or a common pattern of internucleotidic linkage modifications (e.g., of a plurality of oligonucleotide or an oligonucleotide type), or of all oligonucleotides in a composition that share the same constitution, are oligonucleotides of the plurality. In some embodiments, a percentage is at least (DP)^{NCI}, wherein DP is a percentage selected from 85%-100%, and NCI is the number of chirally controlled internucleotidic linkage. In some embodiments, DP is at least 85%, 90%, 91%, 92%, 93%, 94%, 95%, 96%, 97%, 98%, or 99%. In some embodiments, DP is at least 85%. In some embodiments, DP is at least 90%. In some embodiments, DP is at least 95%. In some embodiments, DP is at least 96%. In some embodiments, DP is at least 97%. In some embodiments, DP is at least 98%. In some embodiments, DP is at least 99%. In some embodiments, DP reflects diastereopurity of linkage phosphorus chiral centers chirally controlled internucleotidic linkages. In some embodiments, diastereopurity of a linkage phosphorus chiral center of an internucleotidic linkage may be typically assessed using an appropriate dimer comprising such an internucleotidic linkage and the two nucleoside units being linked by the internucleotidic linkage. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 0.1%-100% (e.g., about 1%-100%, 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. For instance, in some embodiments a chirally controlled oligonucleotide composition comprises one oligonucleotide type at a predetermined level (e.g., as described above). In some embodiments, a chirally controlled oligonucleotide composition comprises more than one oligonucleotide type, each independently at a predetermined level. In some embodiments, a chirally controlled oligonucleotide composition comprises multiple oligonucleotide types, each independently at a predetermined level. In some embodiments, a chirally controlled oligonucleotide composition is a composition of oligonucleotides of an oligonucleotide type, which composition comprises a predetermined level of a plurality of oligonucleotides of the oligonucleotide type.

### DETAILED DESCRIPTION OF CERTAIN EMBODIMENTS

In some embodiments, the present disclosure pertains to, inter alia, oligonucleotide compositions, oligonucleotide drug products, and methods thereof (e.g., WVE-003, an oligonucleotide composition thereof, a chirally controlled oligonucleotide composition thereof, or a therapeutically effective amount thereof, manufacturing methods thereof, therapeutic methods thereof, etc.) for treatment of Huntington's disease (HD) or a symptom thereof.

In some embodiments, the present disclosure pertains to WVE-003, and compositions and methods thereof.

In some embodiments, the present disclosure pertains to WVE-003 or a composition thereof, wherein WVE-003 or the composition is administered in accordance with any of various dosing regimens described herein.

In some embodiments, a dosing regimen pertains to: the amount of an individual dose of an oligonucleotide, an oligonucleotide composition, a chirally controlled oligonucleotide composition, or a therapeutically effective amount of an oligonucleotide, an oligonucleotide composition, a chirally controlled oligonucleotide composition; and/or the interval between multiple or successive doses thereof, and/or the total length or duration of time during which a subject receives one or more doses thereof; and/or a particular formulation thereof.

### Huntingtin (HTT)

In some embodiments, the present disclosure provides technologies, e.g., oligonucleotides, compositions, methods, etc., related to the Huntingtin (HTT) gene or a product encoded thereby (a transcript, a protein (e.g., various variants of the Huntingtin protein, etc.). In some embodiments, the present disclosure provides technologies, including HTT oligonucleotides and compositions and methods thereof, for treatment of Huntington's disease. In some embodiments, HTT comprises one or more mutations. In some embodiments, such mutations are associated with reduced biological functions of Huntingtin protein in a subject suffering from and/or susceptible to Huntington's disease. In some embodiments of Huntington's disease, one or both alleles of HTT are mutant.

In some embodiments, the Huntingtin (HTT) gene or a product thereof, or a variant or portion thereof, may be referred to as HTT.

In some embodiments, HTT refers to a gene or a gene product thereof (including but not limited to, a nucleic acid, including but not limited to a DNA or RNA, or a wild-type or mutant protein encoded thereby), from any species, and which may be also known as: HTT, HD, IT15, huntingtin, Huntingtin, or LOMARS; External IDs: OMIM: 613004, MGI: 96067, HomoloGene: 1593, GeneCards: HTT; Species: Human: Entrez: 3064; Ensembl: ENSG00000197386; UniProt: P42858; RefSeq (mRNA): NM_002111; RefSeq (protein): NP_002102; Location (UCSC): Chr 4: 3.04-3.24 Mb; Species: Mouse: Entrez: 15194; Ensembl: ENSMUSG00000029104; UniProt: P42859; RefSeq (mRNA): NM_010414; RefSeq (protein): NP_034544; Location (UCSC): Chr 5: 34.76-34.91 Mb. Additional HTT sequences, including variants thereof, from human, mouse, rat, monkey, etc., are readily available to those of skill in the art. In some embodiments, HTT is a human or mouse HTT, which is wild-type or mutant.

In some embodiments, a HTT protein is unmodified or modified. In some embodiments, a HTT protein has any one or more modifications of: 9 N6-acetyllysine; 176 N6-acetyllysine; 234 N6-acetyllysine; 343 N6-acetyllysine; 411 Phosphoserine; 417 Phosphoserine; 419 Phosphoserine; 432 Phosphoserine; 442 N6-acetyllysine; 640 Phosphoserine; 643 Phosphoserine; 1179 Phosphoserine; 1199 Phosphoserine; 1870 Phosphoserine; and/or 1874 Phosphoserine.

Without wishing to be bound by any particular theory, the present disclosure notes that a mutation (e.g., a CAG repeat expansion) in HTT is reportedly a key factor in diseases and disorders such as Huntington's disease.

In some embodiments, a mutant HTT is designated mHTT, muHTT, m HTT, mu HTT, MU HTT, or the like, wherein m or mu indicate mutant. In some embodiments, a wild type HTT is designated wild-type HTT, wtHTT, wt HTT, WT HTT, WTHTT, or the like, wherein wt indicates wild-type. In some embodiments, a mutant HTT comprises an expanded CAG repeat region (as appreciated by those skilled in the art, the number of CAG repeats may vary from subject to subject; e.g., in some embodiments, comprising 36 or more CAG repeats). In some embodiments, a mutant HTT comprises a mutant allele of one or more SNP (the allele on the same DNA strand or chromosome as the expanded CAG repeat region). In some embodiments, in a population wild-type HTT are reported to frequently have about or no more than about 18 CAG repeats, and mutant HTT are reported to frequently have about or more than about 43 CAG repeats. In some embodiments, in a population it is reported that heterozygosity of SNP rs362273 was identified in about 71% individuals. In some embodiments, in a population it is reported that SNP3 is associated with mHTT in about 40-45% of patients with HD.

In some embodiments, a mutant HTT comprises both an expanded CAG repeat region and a mutant allele of a particular SNP on the same chromosomal strand. In some embodiments, in such a case, wherein the mutant allele of a particular SNP is targeted by a HTT oligonucleotide (e.g., WVE-003) and the mutant allele is on the same chromosome as the deleterious CAG repeat expansion, the oligonucleotide is capable of targeting the deleterious HTT allele and mediating allele-specific knockdown.

In some embodiments, a human HTT is designated hHTT. In some embodiments, a mutant HTT is designated mHTT. In some embodiments, when a mouse is utilized, a mouse HTT may be referred to as mHTT as those skilled in the art will appreciate.

In some embodiments, an oligonucleotide capable of mediating allele-specific knockdown of a mutant HTT gene or a gene product thereof is WVE-003.

### Huntington's Disease

Compositions comprising one or more HTT oligonucleotides described herein can be used to treat Huntington's disease or a symptom thereof.

Huntington's disease (HD) is reportedly a neurodegenerative disorder reportedly caused by a mutation of the HTT (huntingtin) gene. Alteration of this widely expressed single gene reportedly results in a progressive, neurodegenerative disorder with a large number of characteristic symptoms.

Huntington's disease is reportedly a rare, progressive neurological disease that results in motor, cognitive, and psychiatric disability and is invariably fatal. Bates G, Tabrizi S, Jones L, (editors). Huntington's Disease, 4th Edition. Oxford (UK): Oxford University Press; 2014. Because it is a genetic, hereditary disease, it can reportedly affect multiple family members across generations. Sturrock A et al. J Geriatr Psychiatry Neurol. 2010; 23(4): 243-259. Although cognitive and psychiatric symptoms may reportedly develop first, the clinical diagnosis of HD is usually based on the presence of chorea, one of the most visually prominent symptoms of this disease. Chorea is reportedly an abnormal involuntary movement disorder, which occurs in 90% of subjects and is moderate to severe in approximately 70% of these subjects. These physical symptoms can reportedly appear at any age, but typically appear between the ages of 30 and 50. Bates G, Tabrizi S, Jones L, (editors). Huntington's Disease, 4th Edition. Oxford (UK): Oxford University Press; 2014. A physical examination, sometimes combined with a neurological examination, can reportedly determine whether the onset of the disease has begun. Life expectancy after symptom onset is reduced to around 15 to 20 years. Bates G, Tabrizi S, Jones L, (editors). Huntington's Disease, 4th Edition. Oxford (UK): Oxford University Press; 2014; and Sturrock A et al. J Geriatr Psychiatry Neurol. 2010; 23(4): 243-259. As symptoms progress, individuals reportedly become increasingly or totally dependent on others for care. Suicidal ideation is reportedly increased in the early stages of the disease, likely associated with a perceived loss of independence, and subjects with HD have a significantly higher rate of suicide as compared with a normal healthy adult population (138 of 100,00 persons per year and 12-13 per 100,000 persons per year, respectively). Bird T D. Am J Hum Genet. 1999; 64(5): 1289-1292; and Paulsen J S et al. Am J Psychiatry. 2005; 162(4): 725-731.

Some of the symptoms of HD can reportedly be managed with medication and therapies such as antipsychotics and drugs affecting the dopamine pathways, which modulate the movement disorder. Sturrock A et al. J Geriatr Psychiatry Neurol. 2010; 23(4): 243-259.

Huntington's disease is reportedly caused by a known mutation on a single gene, an expansion of a cytosine-adenine-guanine (CAG) triplet repeat in the Huntingtin (HTT) gene. The Huntington's Disease Collaborative Research Group. Cell. 1993; 72(6): 971-983. Wild type HTT protein is critical for neuronal development. Dragatsis I et al. Nat Genet. 2000; 26(3): 300-306. Although the purpose of wtHTT in adults is reportedly not completely understood, some studies have shown that it may play an important role in neuronal functions. Dragatsis I et al. Nat Genet. 2000; 26(3): 300-306; Leavitt B R et al. J Neurochem. 2006; 96(4): 1121-1129; Rigamonti D et al. J Biol Chem. 2001; 276(18): 14545-14548; and Zhang Y et al. EMBO J. 2006; 25(24): 5896-5906. However, expansion in the CAG triplet repeat in the HTT gene reportedly results in production of the mHTT protein. Accumulation of this protein reportedly leads to progressive loss of neurons in the brain. Sturrock A et al. J Geriatr Psychiatry Neurol. 2010; 23(4): 243-259. In nonclinical studies, lowering the level of mHTT protein as measured in the CSF has reportedly been demonstrated to be therapeutic. DiFiglia et al. Proc Natl Acad Sci USA. 2007; 104(43): 17204-17209; and Kordasiewicz H B et al. Neuron. 2012; 74(6): 1031-1044. Therefore, a drug that can silence the mHTT gene transcript, while leaving the wild type allele intact, may be able to slow, stop, or even reverse the course of HD. Kay C et al. Clin Genet. 2014; 86(1): 29-36.

In some embodiments, a HD-related mutation is an expansion of a CAG repeat region in the HTT gene, wherein a larger expansion reportedly results in greater severity of the disease and an earlier age of onset. The mutation reportedly results in a variety of motor, emotional and cognitive symptoms, and results in the formation of huntingtin aggregates in brain.

The CAG expansion reportedly results in the expansion of a poly-glutamine (poly-Q) tract in the huntingtin protein, a 350 kDa protein (Huntington Disease Collaborative Research Group, 1993. Cell. 72:971-83). A CAG repeat expansion is reportedly associated with Huntington's disease. Longer repeat sequences are reportedly associated with earlier disease onset. The absence of an HD phenotype in individuals deleted for one copy of huntingtin, or increased severity of disease in those homozygous for the expansion reportedly suggests that the mutation does not result in a loss of function (Trottier et al., 1995, Nature Med., 10:104-110). Transcriptional deregulation and loss of function of transcriptional coactivator proteins have reportedly been implicated in HD pathogenesis. Mutant huntingtin has reportedly been shown specifically to disrupt activator-dependent transcription in the early stages of HD pathogenesis (Dunah et al., 2002. Science 296:2238-2243).

In one report gene profiling of human blood identified 322 mRNAs that show significantly altered expression in HD blood samples as compared to normal or presymptomatic individuals. Expression of marker genes was similarly substantially altered in post-mortem brain samples from HD caudate, suggesting that upregulation of genes in blood samples reflects disease mechanisms found in brain. Monitoring of gene expression may provide a sensitive and quantitative method to monitor disease progression, especially in the early stages of disease in both animal models and human subjects (Borovecki et al., 2005, Proc. Natl. Acad. Sci. USA 102:11023-11028).

Huntington's disease has been reported to be an autosomal dominant disorder, with an onset generally in mid-life, although cases of onset from childhood to over 70 years of age have been documented. An earlier age of onset is reportedly associated with paternal inheritance, with 70% of juvenile cases being inherited through the father.

In some embodiments, symptoms of Huntington's disease have an emotional, motor and/or cognitive component. One symptom, chorea, is a characteristic feature of the motor disorder and is defined as excessive spontaneous movements which are irregularly timed, randomly distributed and abrupt. It can vary from being barely perceptible to severe. Other frequently observed abnormalities include dystonia, rigidity, bradykinesia, ocularmotor dysfunction, tremor, etc. Voluntary movement disorders as symptoms include fine motor incoordination, dysarthria, and dysphagia. Emotional disorders commonly include depression and irritability, and cognitive component comprises subcortical dementia (Mangiarini et al. 1996. Cell 87:493-506). It is reported that changes in HD brains are widespread and include neuronal loss and gliosis, particularly in the cortex and striatum (Vonsattel and DiFiglia. 1998. J. Neuropathol. Exp. Neurol. 57:369-384).

In some embodiments, Huntington's disease phenotypes range from mild HTT to severe, depending on the length of the CAG repeat expansion.

Certain information related to HTT and HTT-related conditions, disorders or diseases and symptoms thereof has been reported in the scientific literature, for example: Kremer et al. 1994. N. E. J. Med. 330: 1401; Kordasiewicz et al. 2012 Neuron 74: 1031-1044; Carroll et al. 2011 Mol. Ther. 19: 2178-2185; Warby et al. 2009 Am. J. Hum. Genet. 84: 351-366; Pfister et al. 2009 Current Biol. 19: 774-778; Kay et al. 2015 Mol. Ther. 23: 1759-1771; Kay et al. 2014 Clin. Genet. 86: 29-36; Lee et al. 2015. Am. J. Hum. Genet. 97: 435-444, Skotte et al. 2014. PLOS ONE 9: e107434; Southwell et al. 2014. Mol. Ther. 22: 2093-2106; Australian Pat. Publications AU2017276286 and AU2007210038; European Pat. Publications EP3277814 and EP3210633; International Pat. Publication WO2018145009; and US Pat. Publication US20180273945.

### Treatment of HTT-Related Conditions, Disorders or Diseases

In some embodiments, the present disclosure provides a HTT oligonucleotide which targets HTT (e.g., a HTT oligonucleotide comprising a HTT target sequence or a sequence complementary to a HTT target sequence) and directs target-specific knockdown of HTT, including, for example, WVE-003. In some embodiments, WVE-003 is administered intrathecally in an amount of about 30, about 60, about 90, about 120, about 150 or about 168 mg, and is administered as a liquid formulation (including, but not limited to, a solution in water, or an aCSF solution, or a solution in aCSF reconstituted from a lyophilized preparation or a solution in sodium chloride, or a solution in sodium chloride reconstituted from a lyophilized preparation). In some embodiments, the present disclosure provides a HTT oligonucleotide which directs target-specific knockdown of HTT mediated by RNase H and/or RNA interference.

In some embodiments, the present disclosure provides methods for preventing and/or treating HTT-related conditions, disorders or diseases using provided HTT oligonucleotides and compositions thereof. In some embodiments, a HTT-related condition is Huntington's disease, and/or one or more symptoms of Huntington's disease. In some embodiments, the present disclosure provides oligonucleotides and compositions thereof for use as medicaments, e.g., for HTT-related conditions, disorders or diseases. In some embodiments, the present disclosure provides oligonucleotides and compositions thereof for use in the treatment of HTT-related conditions, disorders or diseases. In some embodiments, the present disclosure provides oligonucleotides and compositions thereof for the manufacture of medicaments for the treatment of HTT-related conditions, disorders or diseases.

In some embodiments, the present disclosure provides a method for preventing, treating or ameliorating a HTT-related condition, disorder or disease in a subject susceptible thereto or suffering therefrom, comprising administering to the subject a therapeutically effective amount of a HTT oligonucleotide or a pharmaceutical composition thereof.

In some embodiments, the present disclosure provides a method for treating or ameliorating a HTT-related condition, disorder or disease in a subject suffering therefrom, comprising administering to the subject a therapeutically effective amount of a HTT oligonucleotide or a pharmaceutical composition thereof. In some embodiments, a HTT oligonucleotide is WVE-003. In some embodiments, a therapeutically effective amount is about 30, about 60, about 90, about 120, about 150 or about 168 mg.

In some embodiments, a HTT-related condition, disorder or disease is Huntington's disease (HD), also known as Huntington's Chorea. In some embodiments, a HTT-related condition, disorder or disease is: juvenile HD, akinetic-rigid, or Westphal variant HD.

In some embodiments, the present disclosure provides a method for reducing HTT gene expression in a cell, comprising: contacting the cell with a HTT oligonucleotide or a composition thereof. In some embodiments, the present disclosure provides a method for reducing the level of a HTT transcript in a cell, comprising: contacting the cell with a HTT oligonucleotide or a composition thereof. In some embodiments, the present disclosure provides a method for reducing the level of a HTT protein in a cell, comprising: contacting the cell with a HTT oligonucleotide or a composition thereof. In some embodiments, provided methods selectively reduce levels of HTT transcripts and/or products encoded thereby that are related to conditions, disorders or diseases.

Reportedly, HTT is expressed in all cells, with the highest concentrations are found in the brain and testes, with moderate amounts in the liver, heart, and lungs. In various embodiments, a cell is in brain, testes, liver, heart, or lungs.

In some embodiments, the present disclosure provides a method for decreasing HTT gene expression in a mammal in need thereof, comprising administering to the mammal a nucleic acid-lipid particle comprising a provided HTT oligonucleotide or a composition thereof.

In some embodiments, the present disclosure provides a method for in vivo delivery of a HTT oligonucleotide, comprising administering to a mammal a HTT oligonucleotide or a composition thereof.

In some embodiments, a mammal is a human. In some embodiments, a mammal is afflicted with, suffering from and/or susceptible to a HTT-related condition, disorder or disease.

In some embodiments, a subject or subject suitable for treatment of a HTT-related condition, disorder or disease, such as Huntington's disease (HD), can be identified or diagnosed by a health care professional. For example, for a neurological condition, disorder or disease, a physical exam may be followed by a thorough neurological exam. In some embodiments, an neurological exam may assess motor and sensory skills, nerve function, hearing and speech, vision, coordination and balance, mental status, and/or changes in mood or behavior. Example symptoms of neurological conditions, disorders or diseases, such as Huntington's disease (HD), include weakness in the arms, legs, feet, or ankles; slurring of speech; difficulty lifting the front part of the foot and toes; hand weakness or clumsiness; muscle paralysis; rigid muscles; involuntary jerking or writing movements (chorea); involuntary, sustained contracture of muscles (dystonia); bradykinesia; loss of automatic movements; impaired posture and balance; lack of flexibility; tingling parts in the body; electric shock sensations that occur with movement of the head; twitching in arm, shoulders, and tongue; difficulty swallowing; difficulty breathing; difficulty chewing; partial or complete loss of vision; double vision; slow or abnormal eye movements; tremor; unsteady gait; fatigue; loss of memory; dizziness; difficulty thinking or concentrating; difficulty reading or writing; misinterpretation of spatial relationships; disorientation; depression; anxiety; difficulty making decisions and judgments; loss of impulse control; difficulty in planning and performing familiar tasks; aggressiveness; irritability; social withdrawal; mood swings; dementia; change in sleeping habits; wandering; and/or change in appetite.

In some embodiments, a symptom of Huntington's disease is any of insoluble protein accumulation; huntingtin protein aggregate accumulation; neuronal aggregates in the striatum; alteration in the size and number of neuronal intranuclear inclusions and other markers of HD; alteration in regulation of DARPP-32 expression; striatal atrophy; striatal and cortical neurodegeneration; alteration of blood glucose and/or insulin levels; or neuronal loss and gliosis, particularly in the cortex and striatum.

In some embodiments, a symptom of Huntington's disease is any of behavioral and neuropathological abnormalities; in test animals, altered rotarod performance; reduction of weight loss; alteration of lifespan; behavioral disturbance; emotional, motor and cognitive alterations or impairment; depression; irritability; involuntary movements (chorea); choreiform movements; impaired coordination; excessive spontaneous movements which are irregularly timed, randomly distributed and abrupt; bradykinesia; dystonia; seizures; rigidity; ocularmotor dysfunction; tremor; fine motor incoordination; dysarthria; dysphagia; subcortical dementia; progressive dementia; or psychiatric disturbance.

In some embodiments, a provided oligonucleotide or a composition thereof prevents, treats, ameliorates, or slows progression of a HTT-related condition, disorder or disease, or at least one symptom of a HTT-related condition, disorder or disease. In some embodiments, a provided oligonucleotide or a composition thereof prevents, treats, ameliorates, or slows progression of a HTT-related condition, disorder or disease, or two or more symptoms of a HTT-related condition, disorder or disease. In some embodiments, a subject is assessed for one or more marker and/or symptoms of a HTT-related condition, disorder or disease, wherein the subject is subsequently prescribed and/or given an oligonucleotide or oligonucleotide administration (e.g., a dose or a dosage regimen of an oligonucleotide) as described herein. In some embodiments, a subject is assessed for one or more marker and/or symptoms of a HTT-related condition, disorder or disease, wherein the subject has been given an oligonucleotide or an oligonucleotide administration as described herein. In some embodiments, a HTT-related condition, disorder or disease is Huntington's disease.

In some embodiments, a method of the present disclosure is for the treatment of Huntington's disease in a subject wherein the method comprises administering to a subject a therapeutically effective amount of a HTT oligonucleotide or a pharmaceutical composition thereof. In some embodiments, the HTT oligonucleotide is WVE-003.

In some embodiments, a subject has an allele or transcript that comprises an expanded CAG repeat region and is fully complementary to the base sequence of WVE-003. In some embodiments, HTT transcripts comprising an expanded CAG repeat region are fully complementary to the base sequence of WVE-003. In some embodiments, a subject has an allele that does not contain an expanded CAG repeat region and is fully complementary to the base sequence of WVE-003. In some embodiments, a subject has a HTT allele that does not contain an expanded CAG repeat region and is not complementary to the base sequence of WVE-003 at rs362273. In some embodiments, HTT transcripts that do not contain an expanded CAG repeat region are not fully complementary to the base sequence of WVE-003 at rs362273.

In some embodiments, a provided method reduces at least one symptom of Huntington's disease wherein the method comprises administering to a subject a therapeutically effective amount of a HTT oligonucleotide or a pharmaceutical composition thereof.

In some embodiments, the present disclosure provides a method for treating and/or ameliorating one or more symptoms associated with a HTT-related condition, disorder or disease in a mammal in need thereof, the method comprising administering to the mammal a therapeutically effective amount of a HTT oligonucleotide or a composition thereof. In some embodiments, the present disclosure provides a method for reducing susceptibility to a HTT-related condition, disorder or disease in a mammal in need thereof, the method comprising: administering to the mammal a therapeutically effective amount of a HTT oligonucleotide or a composition thereof. In some embodiments, the present disclosure provides a method for preventing or delaying the onset of a HTT-related condition, disorder or disease in a mammal in need thereof, the method comprising: administering to the mammal a therapeutically effective amount of a HTT oligonucleotide or a composition thereof. In some embodiments, the present disclosure provides a method for treating and/or ameliorating one or more symptoms associated with a HTT-related condition, disorder or disease in a mammal in need thereof, the method comprising: administering to the mammal a therapeutically effective amount of a nucleic acid-lipid particle comprising a HTT oligonucleotide. In some embodiments, the present disclosure provides a method for reducing susceptibility to a HTT-related condition, disorder or disease in a mammal in need thereof, the method comprising: administering to the mammal a therapeutically effective amount of a nucleic acid-lipid particle comprising a HTT oligonucleotide. In some embodiments, the present disclosure provides a method for preventing or delaying the onset of a HTT-related condition, disorder or disease in a mammal in need thereof, the method comprising: administering to the mammal a therapeutically effective amount of a nucleic acid-lipid particle comprising a HTT oligonucleotide. In some embodiments, a mammal is a human. In some embodiments, a mammal is afflicted with and/or suffering from and/or susceptible to a HTT-related condition, disorder or disease. In some embodiments, a HTT oligonucleotide is WVE-003. In some embodiments, a therapeutically effective amount is about 30, about 60, about 90, about 120, about 150 or about 168 mg.

In some embodiments, the present disclosure pertains to compositions and methods related to specific doses of WVE-003 (or a salt form thereof) which are about 30 mg, about 60 mg, about 90 mg, about 120 mg, about 150 mg, or about 168 mg.

In some embodiments, about 30 mg is 30 mg±5%, about 60 mg is 60 mg±5%, about 90 mg is 90 mg±5%, about 120 mg is 120 mg±5%, about 150 mg is 150 mg±5% and/or about 168 mg is 168 mg±5%.

In some embodiments, about 30 mg is 30 mg-10%, about 60 mg is 60 mg±10%, about 90 mg is 90 mg 10%, about 120 mg is 120 mg±10%, about 150 mg is 150 mg±10% and/or about 168 mg is 168 mg±10%.

In some embodiments, about 30 mg is 30 mg±15%, about 60 mg is 60 mg±15%, about 90 mg is 90 mg±15%, about 120 mg is 120 mg 15%, about 150 mg is 150 mg±15% and/or about 168 mg is 168 mg±15%.

In some embodiments, about 30 mg is 30 mg±20%, about 60 mg is 60 mg±20%, about 90 mg is 90 mg 20%, about 120 mg is 120 mg±20%, about 150 mg is 150 mg 20% and/or about 168 mg is 168 mg±20%.

In some embodiments, about 30 mg is 30 mg±25%, about 60 mg is 60 mg±25%, about 90 mg is 90 mg 25%, about 120 mg is 120 mg±25%, about 150 mg is 150 mg 25% and/or about 168 mg is 168 mg±25%.

In some embodiments, about 30 mg is 30 mg±30%, about 60 mg is 60 mg±30%, about 90 mg is 90 mg±30%, about 120 mg is 120 mg±30%, about 150 mg is 150 mg 30% and/or about 168 mg is 168 mg±30%.

In some embodiments, about 30 includes but is not limited to: 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38, 38.1, 38.2, 38.3, 38.4, 38.5, 38.6, and 38.7.

In some embodiments, about 30 includes but is not limited to: 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, and 40.

In some embodiments, about 30 includes but is not limited to: 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, and 35.2.

In some embodiments, the present disclosure pertains to compositions and methods related to specific dose of WVE-003 (or a salt form thereof) which are about 30 mg, about 60 mg, about 90 mg, about 120 mg, about 150 mg, or about 168 mg.

In some embodiments, the present disclosure pertains to compositions and methods related to specific doses of WVE-003 (or a salt form thereof) which are about 30 mg, about 60 mg, about 90 mg, about 120 mg, about 150 mg, or about 168 mg, wherein the total amount of oligonucleotide in the dose is about 30 mg, about 60 mg, about 90 mg, about 120 mg, about 150 mg, or about 168 mg, respectively, and substantially all of the oligonucleotide in the dose is WVE-003.

In some embodiments, the present disclosure provides WVE-003 preparations of high purity. Various technologies are available for assess WVE-003 purity. In some embodiments, purity is assessed using Protocol A as described herein. In some embodiments, purity of a WVE-003 preparation is about 80% or more. In some embodiments, it is about 81%. In some embodiments, it is about 82%. In some embodiments, it is about 83%. In some embodiments, it is about 84%. In some embodiments, it is about 85%. In some embodiments, it is about 86%. In some embodiments, it is about 87%. In some embodiments, it is about 88%. In some embodiments, it is about 89%. In some embodiments, it is about 90%. In some embodiments, it is about 90% or more. In some embodiments, various batches of WVE-003 were found to be, for example, about 84%-88% pure, with about 11%-16% total being impurities. In some embodiments, impurities include n−x deletion sequences; n−1 deletion sequences and phosphodiesters; modified full length sequences and/or n+x addition sequences. Without wishing to be bound by any particular theory, this disclosure notes that at least some of the impurities will have at least some activity (e.g., capability to mediate allele-specific knockdown of a mutant HTT gene or gene product).

In some embodiments, WVE-003 is administered to a subject at a dose of about 30, about 60, about 90, about 120, about 150 or about 168 mg. In some embodiments, multiple doses of WVE-003 are administered to a subject. In some embodiments, multiple doses of WVE-003 are administered to a subject at regular intervals. In some embodiments, multiple doses of WVE-003 are administered to a subject approximately monthly (e.g., with an interval of about 1 month between dosages). In some embodiments, multiple doses of WVE-003 are administered to a subject approximately once every 2 months (e.g., with an interval of about 2 months between dosages). In some embodiments, multiple doses of WVE-003 are administered to a subject approximately once approximately every 8 weeks (e.g., with an interval of about 8 weeks between dosages). In some embodiments, multiple doses of WVE-003 are administered to a subject approximately once approximately every 12 weeks (e.g., with an interval of about 12 weeks between dosages). In some embodiments, each of the multiple doses is about the same, e.g., about 30 mg.

In some embodiments, multiple doses of WVE-003 are administered to a subject once approximately every 4 weeks (e.g., with an interval of approximately 4 weeks between dosages). In some embodiments, multiple doses of WVE-003 are administered to a subject once approximately every 8 weeks (e.g., with an interval of approximately 8 weeks between dosages). In some embodiments, multiple doses of WVE-003 are administered to a subject once approximately every 12 weeks (e.g., with an interval of approximately 12 weeks between dosages).

In some embodiments, the subject is administered two or more doses of WVE-003, and the interval between any two of the doses is about a month or about 4 weeks. In some embodiments, the subject is administered two or more doses of WVE-003, and the interval between any two of the doses is about 2 months or about 8 weeks. In some embodiments, the subject is administered two or more doses of WVE-003, and the interval between any two of the doses is about 3 months or about 12 weeks.

In some embodiments, the subject is administered two or more doses of WVE-003, approximately monthly or approximately once every 4 weeks. In some embodiments, the subject is administered two or more doses of WVE-003, approximately once every 2 months or approximately once every 8 weeks. In some embodiments, the subject is administered two or more doses of WVE-003, approximately once every 3 months or approximately once every 12 weeks.

In some embodiments, the subject is administered WVE-003 approximately monthly for at least about 2 months. In some embodiments, the subject is administered WVE-003 approximately once every two months for at least about 2 months.

In some embodiments, the subject is administered WVE-003 approximately monthly for at least about 4 months. In some embodiments, the subject is administered WVE-003 approximately once every two months for at least about 4 months.

In some embodiments, the subject is administered WVE-003 approximately monthly for at least about 8 months. In some embodiments, the subject is administered WVE-003 approximately once every two months for at least about 8 months.

In some embodiments, the subject is administered WVE-003 approximately once every 4 weeks for at least about 8 weeks. In some embodiments, the subject is administered WVE-003 approximately once every 8 weeks for at least about 16 weeks.

In some embodiments, the subject is administered WVE-003 approximately once every 4 weeks for at least about 12 weeks. In some embodiments, the subject is administered WVE-003 approximately once every 12 weeks for at least about 12 weeks.

In some embodiments, the subject is administered WVE-003 approximately once every 4 weeks for at least about 16 weeks. In some embodiments, the subject is administered WVE-003 approximately once every 8 weeks for at least about 16 weeks.

In some embodiments, the subject is administered WVE-003 approximately once every 8 weeks for at least about 24 weeks. In some embodiments, the subject is administered WVE-003 approximately once every 12 weeks for at least about 24 weeks.

In some embodiments, administration of a HTT oligonucleotide to a subject or patient is capable of mediating any one or more of: slowing Huntington's disease progression, delaying the onset of HD or at least one symptom thereof, improving one or more indicators of HD, and/or increasing the survival time or lifespan of the subject or patient.

In some embodiments, slowing disease progression relates to the prevention of, or delay in, a clinically undesirable change in one or more clinical parameters in an individual and/or susceptible to HD, such as those described herein. It is well within the abilities of a physician to identify a slowing of disease progression in an individual suffering from HD, using one or more of the disease assessment tests described herein. Additionally, it is understood that a physician may administer to the individual diagnostic tests other than those described herein to assess the rate of disease progression in an individual suffering from HD.

In some embodiments, delaying the onset of HD or a symptom thereof relates to delaying one or more undesirable changes in one or more indicators of HD that are negative for HD. A physician may use family history of HD or comparisons to other HD patients (e.g., subjects, or human beings receiving or in need of treatment for HD) with similar genetic profile (e.g., number of CAG repeats) to determine an expected approximate age of HD onset to HD to determine if onset of HD is delayed.

In some embodiments, indicators of HD include parameters employed by a medical professional, such as a physician, to diagnose or measure the progression of HD, and include, without limitation, genetic testing, hearing, eye movements, strength, coordination, chorea (rapid, jerky, involuntary movements), sensation, reflexes, balance, movement, mental status, dementia, personality disorder, family history, weight loss, and degeneration of the caudate nucleus. Degeneration of the caudate nucleus is assessed via brain imaging techniques such as magnetic resonance imaging (MRI) or computed tomography (CT) scan.

In some embodiments, an improvement in an indicator of HD relates to the absence of an undesirable change, or the presence of a desirable change, in one or more indicators of HD. In one embodiment, an improvement in an indicator of HD is evidenced by the absence of a measurable change in one or more indicators of HD. In another embodiment, an improvement in an indicator of HD is evidenced by a desirable change in one or more indicators of HD.

In some embodiments, a slowing of disease progression may further comprise an increase in survival time in an individual suffering from and/or susceptible to HD. In some embodiments, an increase in survival time relates to mean increasing the survival of an individual suffering from and/or susceptible to HD, relative to an approximate survival time based upon HD progression and/or family history of HD. A physician can use one or more of the disease assessment tests described herein to predict an approximate survival time of an individual suffering from and/or susceptible to HD. A physician may additionally use the family history of an individual suffering from and/or susceptible to HD or comparisons to other HD patients with similar genetic profile (e.g., number of CAG repeats) to predict expected survival time.

In some embodiments, the present disclosure provides a method of inhibiting HTT expression in a cell, the method comprising: (a) contacting the cell with a HTT oligonucleotide; and (b) maintaining the cell produced in step (a) for a time sufficient to obtain degradation of a mRNA transcript of a HTT gene, thereby inhibiting expression of the HTT gene in the cell. In some embodiments, HTT expression is inhibited by at least 30%.

In some embodiments, the present disclosure provides a method of treating a condition, disorder or disease mediated by HTT expression comprising administering to a human suffering therefrom a therapeutically effective amount of a HTT oligonucleotide or a composition thereof. In some embodiments, administration causes a decrease in the expression, activity and/or level of a HTT transcript. In some embodiments, administration is associated with a decrease in the expression, activity and/or level of a HTT transcript. In some embodiments, administration is followed by a decrease in the expression, activity and/or level of a HTT transcript.

In some embodiments, the present disclosure provides a HTT oligonucleotide for use in a subject to treat a HTT-related condition, disorder or disease. In some embodiments, a HTT-related condition, disorder or disease is Huntington's disease.

In some embodiments, a provided method reduces amount and/or percentage of mHTT protein. In some embodiments, a provided method reduces amount and/or percentage of mHTT protein in CSF. In some embodiments, a provided method increases percentage of wtHTT protein. In some embodiments, a provided method increases percentage of wtHTT protein in CSF. In some embodiments, a provided method does not reduce amount of wtHTT protein, or does not reduce amount of wtHTT protein by more than about 5%, 10%, 15%, 20%, 25%, 30%, 35%, 40%, 45%, or 50%. In some embodiments, a provided method does not reduce amount of wtHTT protein, or does not reduce amount of wtHTT protein by more than about 5%, 10%, 15%, 20%, 25%, 30%, 35%, 40%, 45%, or 50% in CSF. In some embodiments, amount and/or percentage of mHTT, wtHTT and/or total HTT are independently assessed after a suitable period of time (e.g., 1, 2, 3, 4, 5, 6, 7, 8, 9, or 10 days, or 1, 2, 3, 4, 5, 6, 7, 8, 9, or 10 weeks, or 1, 2, 3, 4, 5, 6, 7, 8, 9, or 10 months) after one or more doses each independently as described herein. Certain useful technologies for assessing mHTT, wtHTT and/or total HTT proteins are described in WO 2022/046723 and are incorporated herein by reference.

In some embodiments, a subject is administered an oligonucleotide, e.g., a HTT oligonucleotide (e.g., WVE-003), or a salt form thereof, or a composition thereof and an additional agent and/or method, e.g., an additional therapeutic agent and/or method. In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components are administered as one composition. In some embodiments, at a time point a subject being administered is exposed to both provided oligonucleotides and additional components at the same time.

### Allele-Specific Knockdown of a Mutant HTT Transcript as a Treatment for Huntington's Disease

In some embodiments, a treatment for Huntington's disease comprises the use of a HTT oligonucleotide which is capable of mediating allele-specific knockdown of a mutant HTT transcript. In some embodiments, the present disclosure provides methods for treatment of Huntington's disease comprising the step of administering to a subject suffering therefrom or susceptible thereto a HTT oligonucleotide, or a composition comprising a HTT oligonucleotide. Particularly, among other things, the present disclosure demonstrates that chirally controlled oligonucleotide/chirally controlled oligonucleotide compositions are unexpectedly effective for modulating allele-specific knockdown of a mutant HTT transcript compared to otherwise identical but non-chirally controlled oligonucleotide/oligonucleotide compositions. In some embodiments, the term chirally controlled can be equated with stereopure or stereodefined.

In some embodiments, a treatment for Huntington's disease employs the use of a HTT oligonucleotide, wherein the oligonucleotide is capable of providing allele-specific knockdown of a mutant HTT transcript. In some embodiments, a HTT oligonucleotide is capable of mediating the allele-specific knockdown of a mutant HTT transcript which comprises a mutation (e.g., CAG repeat expansion), wherein a decrease in the level, expression and/or activity of mHTT is capable of treating, preventing, and/or ameliorating Huntington's disease or a symptom thereof, and/or decreasing the severity of or delaying the onset of a symptom of Huntingtin's Disease.

In some embodiments, a composition comprising a HTT oligonucleotide is useful for treatment of a Huntingtin-related disorder of the central nervous system. In some embodiments, the present disclosure provides a method of treatment of a Huntingtin-related disorder of the central nervous system, wherein the method comprises the step of administering a therapeutically effective amount of a HTT oligonucleotide to a subject suffering from a Huntingtin-related disorder of the central nervous system. In some embodiments, a HTT oligonucleotide is administered outside the central nervous system (as non-limiting examples, intrathecally or intramuscularly) to a subject suffering from and/or susceptible to a Huntingtin-related disorder of the central nervous system, and the HTT oligonucleotide is capable of passing through the blood-brain barrier into the central nervous system. In some embodiments, a HTT oligonucleotide is administered directly into the central nervous system (as non-limiting example, via intrathecal, intraventricular, intracranial, etc., delivery).

In some embodiments, in a HTT subject, a HTT gene or transcript has a CAG repeat expansion.

In some embodiments, a HTT subject has a CAG repeat expansion. In some embodiments, allele-specific knockdown of a mutant HTT transcript can be employed to remove or reduce effects of such a mutation by decreasing the level, expression and/or activity of a protein expressed from a mHTT transcript.

In some embodiments, a HTT subject or a subject suspected to have HTT is analyzed for HTT genotype prior to administration of a composition comprising a HTT oligonucleotide.

In some embodiments, a HTT subject or a subject suspected to have HTT is analyzed for HTT phenotype prior to administration of a composition comprising a HTT oligonucleotide.

In some embodiments, a HTT subject is analyzed for genotype and phenotype to determine the relationship of HTT genotype and HTT phenotype prior to administration of a composition comprising a HTT oligonucleotide.

In some embodiments, a subject is genetically verified to have Huntington's disease prior to administration of a composition comprising a HTT oligonucleotide.

In some embodiments, analysis of HTT genotype or genetic verification of mHTT in a subject comprises determining if the subject has one or more deleterious mutations in HTT.

In some embodiments, analysis of HTT genotype or genetic verification of mHTT in a subject comprises determining if the subject has, on the same chromosome, a CAG repeat expansion and a SNP targeted by a particular HTT oligonucleotide; in some embodiments, such an analysis is referenced as phasing.

In some embodiments, a target nucleic acid sequence and a reference nucleic acid sequence differs at one or more sites, e.g., a mutation site, a single-nucleotide polymorphism (SNP) site, etc. In some embodiments, a target nucleic acid sequence and a reference nucleic acid sequence comprise a difference at a SNP site. In some embodiments, a site in a target nucleic acid is fully complementary to a site in an oligonucleotide of the present disclosure while the corresponding site in a reference nucleic acid is not.

In some embodiments, analysis of HTT genotype or genetic verification of mHTT informs the selection of a composition comprising a HTT oligonucleotide useful for treatment.

In some embodiments, an abnormal or mutant HTT gene or a portion thereof is removed or copied from a subject or a subject's cell(s) or tissue(s) and the abnormal or mutant HTT gene, or a portion thereof comprising the abnormality or mutation, or a copy thereof, is inserted into a cell. In some embodiments, this cell can be used to test various compositions comprising a HTT oligonucleotide to predict if such a composition would be useful as a treatment for the subject. In some embodiments, the cell is a myoblast or myotubule.

In some embodiments, prior to a subject receiving treatment with a HTT oligonucleotide, phasing is performed on the subject's genome to determine if the CAG repeat expansion is on the same chromosome as a SNP targeted by a HTT oligonucleotide capable of mediating allele-specific knockdown.

In some embodiments, a HTT oligonucleotide capable of mediating allele-specific knockdown of a mutant HTT gene or gene product thereof, and useful for methods of treatment of Huntington's disease is WVE-003.

### WVE-003

In some embodiments, the present disclosure provides WVE-003, and preparations and compositions thereof. In some embodiments, the present disclosure provides technologies for manufacturing WVE-003. In some embodiments, the present disclosure provides technologies for assessing and/or characterizing WVE-003. In some embodiments, the present disclosure provide technologies for assessing purity of WVE-003, e.g., Protocol A. In some embodiments, the present disclosure provide technologies for confirming stereochemical identity of WVE-003 (or a stereoisomer thereof (e.g., with respect to chiral linkage phosphorus)), e.g., using IP-RP-UPLC (e.g., according to Protocol B). In some embodiments, the present disclosure provide technologies for assessing stereopurity of WVE-003, e.g., using IP-RP-UPLC (e.g., according to Protocol B), dimer modeling, etc. In some embodiments, the present disclosure provides methods for using WVE-003. For example, in some embodiments, the present disclosure provides methods for treating Huntington's disease, comprising administering to a subject suffering therefrom an amount of WVE-003 as described herein.

In some embodiments, a therapeutic uses of oligonucleotides include modulating the function of target mHTT RNAs to reduce the production of disease-associated mHTT proteins. In some embodiments, the mechanism of action used by many oligonucleotides, including antisense oligonucleotides, is to promote degradation of the target mHTT RNA. In some embodiments, modifications of phosphodiester linkages, e.g., use of phosphorothioate linkages, improve the stability, biodistribution, and cellular uptake of oligonucleotides. The use of chiral internucleotidic linkages in oligonucleotide synthesis can create a chiral center at the linkage phosphorus. A chiral linkage phosphorus center can have either an “Sp” or “Rp” configuration. A conventional stereorandom preparation of an oligonucleotide that contains n chiral linkage phosphorus is a mixture of 2ⁿ stereoisomers with respect to chiral linkage phosphorus centers, each of which stereoisomers share the same constitution but differs in stereochemistry along its backbone. For an oligonucleotide in which there are 17 chiral linkage phosphorus centers, a stereorandom preparation can have over 131,000 (2¹⁷) stereoisomers with respect to chiral linkage phosphorus, each of which stereoisomers exists at a very low level (˜1/131000).

Contrary to stereorandom preparations, WVE-003 utilized herein are prepared stereoselectively. As described herein, in some embodiments, each chiral internucleotidic linkage is independently formed with about 97% or more diastereoselectivity (e.g., as measured through preparation of a suitable dimer). In some embodiments, most chiral internucleotidic linkages are independently formed with about 98% or more diastereoselectivity. In some embodiments, one or more, e.g., 1, 2, 3, 4, 5, 6, 7, 8 or more chiral internucleotidic linkages are independently formed with about 99% or more diastereoselectivity. In some embodiments, overall diastereoselectivity (as product of diastereoselectivity of all chiral internucleotidic linkages) is about 80% or more. In some embodiments, it is about 81% or more. In some embodiments, it is about 82% or more. In some embodiments, it is about 83% or more. In some embodiments, it is about 84% or more. In some embodiments, it is about 85% or more.

In some embodiments, an oligonucleotide is WVE-003 which is a stereodefined oligonucleotide that can selectively target mHTT, leaving wtHTT relatively unaffected. WVE-003 can specifically targets the mHTT mRNA transcript, at the A variant of SNP rs362273 (SNP3). A SNP is a single variation that in some instances can be associated with a mutated gene. One of the most frequent SNPs in the mHTT gene is SNP3, which has been reported to be present in approximately 40% to 45% of patients with HD. Kay C et al. Clin Genet. 2014; 86(1): 29-36; Kay C et al. Mol Ther. 2015; 23(11): 1759-1771; Pfister E L et al. Curr Biol. 2009; 19(9): 774-778. By selectively targeting the SNP3 variant associated with the pathogenic CAG expansion (≥36 repeats), administration of WVE-003 may result in selective reduction of the level of mHTT protein.

In some embodiments, WVE-003 is utilized as a disease-modifying agent for the treatment of subjects with Huntington's disease (HD). In some embodiments, WVE-003 is a stereopure antisense oligonucleotide (ASO) utilized to selectively target the mutant form of the huntingtin (mHTT) gene transcript.

In some embodiments, a HTT oligonucleotide, or a salt form thereof, is WVE-003 or a salt form thereof. In some embodiments, an oligonucleotide composition comprises WVE-003 or a salt form thereof.

The base sequence of WVE-003 is 5′-GUUGATCTGTAGCAGCAGCT-3′.

In some embodiments, WVE-003 may be described as: 5′-mG*SmUn001RmUmGn001RmA*ST*SC*ST*SG*ST*RA*SG*SC*SA*SG*Rm5Ceon001RAeoGeon0 01Rm5Ceo*STeo-3′, wherein:
*S represents a Sp phosphorothioate linkage;
*R represents a Rp phosphorothioate linkage;
mX represents 2′-O-methylribonucleoside;
X represents 2′-deoxyribonucleoside;
Xeo represents 2′-O-(2-methoxyethyl)ribonucleoside;
m5Ceo represents 2′-O-(2-methoxyethyl)-5-methylcytidine;
n001R represents Rp N-(1,3-dimethylimidazolidin-2-ylidene) phosphoramidate diester linkage (PN); and
no “*R”, “*S”, or “n001R” between nucleosides represents a natural phosphate linkage (PO).

In some embodiments, WVE-003 is in the form of a sodium salt. In some embodiments, a sodium salt of WVE-003 may be described as: [P(S)]-2′-O-methyl-P-thioguanylyl-(3′→5′)-[P(R)]-2′-O-methyl-P-deoxy-P-[(1,3-dimethylimidazolidin-2-ylidene)amino]uridylyl-(3′→5′)-2′-O-methyluridylyl-(3′→5′)-[P(R)]-2′-O-methyl-P-deoxy-P-[(1,3-dimethylimidazolidin-2-ylidene)amino]guanylyl-(3′→5′)-[P(S)]-2′-O-methyl-P-thioadenylyl-(3′→5′)-[P(S)]-P-thiothymidylyl-(3′→5′)-[P(S)]-2′-deoxy-P-thiocytidylyl-(3′→5′)-[P(S)]-P-thiothymidylyl-(3′→5′)-[P(S)]-2′-deoxy-P-thioguanylyl-(3′→5′)-[P(R)]-P-thiothymidylyl-(3′→5′)-[P(S)]-2′-deoxy-P-thioadenylyl-(3′→5′)-[P(S)]-2′-deoxy-P-thioguanylyl-(3′→5′)-[P(S)]-2′-deoxy-P-thiocytidylyl-(3′→5′)-[P(S)]-2′-deoxy-P-thioadenylyl-(3′→5′)-[P(R)]-2′-deoxy-P-thioguanylyl-(3′→5′)-[P(R)]-2′-O-(2-methoxyethyl)-P-deoxy-P-[(1,3-dimethylimidazolidin-2-ylidene)amino]-5-methylcytidylyl-(3′→5′)-2′-O-(2-methoxyethyl)adenylyl-(3′→5′)-[P(R)]-2′-O-(2-methoxyethyl)-P-deoxy-P-[(1,3-dimethylimidazolidin-2-ylidene)amino]guanylyl-(3′→5′)-[P(S)]-2′-O-(2-methoxyethyl)-P-thio-5-methylcytidylyl-(3′→5′)-2′-O-(2-methoxyethyl)-5-methyluridine, pentadecasodium salt.

In some embodiments, a structure of a WVE-003 sodium salt is presented as below: As appreciated by those skilled in the art, in an acid form, each of Na⁺ is replaced with H⁺. The zig-zag lines represent the linkage between oxygen at 3′ to the phosphorous in the internucleotide linkage.

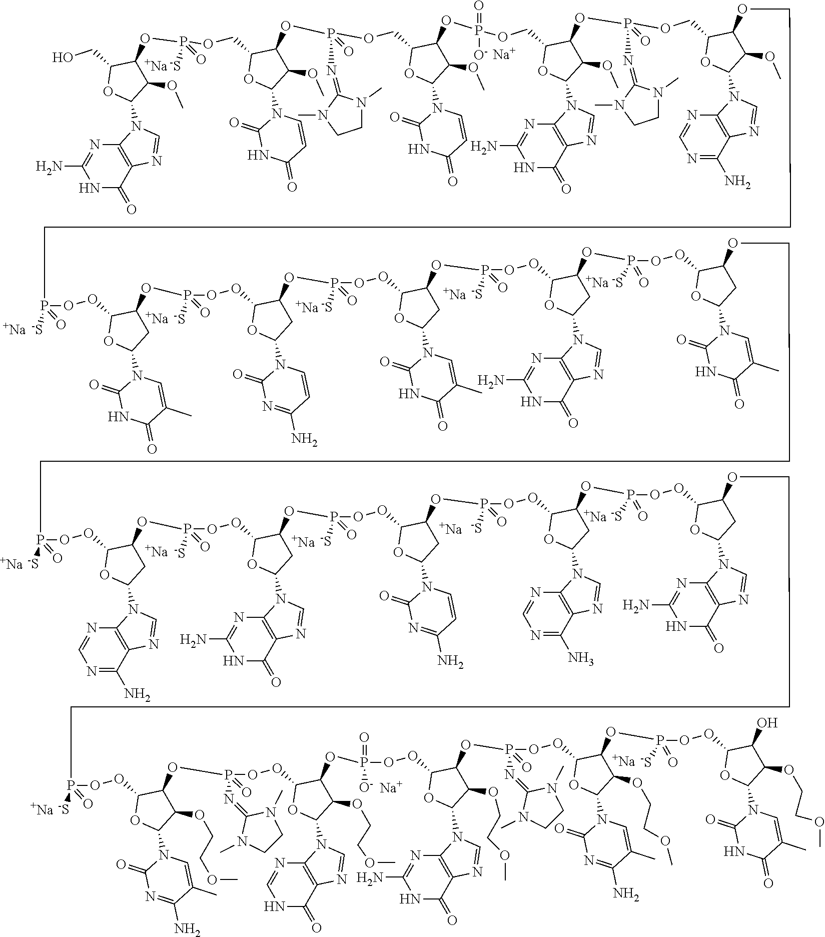

Certain information of WVE-003:
Molecular Formula (sodium salt form): C₂₃₆H₃₀₈N₈₆Na₁₅O₁₁₈P₁₉S₁₃
Molecular Weight (sodium salt form): 7587.67 g/mol
Molecular Formula (free acid): C₂₃₆H₃₂₃N₈₆O₁₁₈P₁₉S₁₃
Molecular Weight (free acid): 7257.94 g/mol
WVE-003 has 19 internucleotide linkages 2 of which are phosphodiester linkages and 17 stereodefined internucleotide linkages, 11 of which are Sp phosphorothioate diester, 2 of which are Rp phosphorothioate diester, and 4 of which are Rp N-(1,3-dimethylimidazolidin-2-ylidene) phosphoramidate diester. This combination of internucleotide linkages can be illustrated with the following sequence of letters: 5′-SnROnRSSSSSRSSSSRnROnRS-3′, where ‘S’, ‘R’, “nR”, and ‘O’ represent Sp phosphorothioate diester, Rp phosphorothioate diester, Rp N-(1,3-dimethylimidazolidin-2-ylidene) phosphoramidate diester and phosphodiester linkages, respectively.

In some embodiments, WVE-003 recognizes the disease-associated (e.g., mutant) allele of SNP rs362273 in the Huntingtin gene, is efficacious in reducing the level, expression and/or activity of a mHTT gene (or a gene product thereof), and is capable of mediating allele-specific knockdown of the mutant Huntingtin (mHTT) gene.

In some embodiments, the efficacy and allele-specificity of chirally controlled WVE-003 composition is superior to that of various stereorandom oligonucleotide compositions.

For additional information related to WVE-003, see, for example, WO 2020/227691 and WO 2021/071788, the entirety of each of which is incorporated herein by reference.

### HTT Oligonucleotides and HTT Oligonucleotide Compositions

In some embodiments, a HTT oligonucleotide, or a salt form thereof, or a HTT oligonucleotide composition (including but not limited to WVE-003) is capable of mediating an allele-specific decrease in the level, expression and/or activity of a mHTT transcript. In some embodiments, a HTT oligonucleotide, or a salt form thereof, or a HTT oligonucleotide composition is capable of mediating allele-specific knockdown of a mutant HTT transcript. In some embodiments, a HTT oligonucleotide, or a salt form thereof, or a HTT oligonucleotide composition is capable of mediating an allele-specific decrease in the level, expression and/or activity of a mHTT transcript. In some embodiments, a HTT oligonucleotide, or a salt form thereof, or a HTT oligonucleotide composition is chirally controlled.

In some embodiments, a HTT oligonucleotide, or a salt form thereof, or a HTT oligonucleotide composition is or comprises WVE-003. In some embodiments, a HTT oligonucleotide, or a salt form thereof, or a HTT oligonucleotide composition is capable of mediating an allele-specific decrease in the level, expression and/or activity of mHTT and is chirally controlled. When administered as described herein, WVE-003 is capable of mediating allele-specific knockdown of a mutant HTT transcript and a potential disease-modifying therapy for Huntington's disease. In some embodiments, a HTT oligonucleotide, or a salt form thereof, or a HTT oligonucleotide composition is capable of mediating an allele-specific decrease in the level, expression and/or activity of mHTT and is chirally controlled.

In some embodiments, the present disclosure provides methods of use of a HTT oligonucleotide or a HTT oligonucleotide composition which is capable of mediating allele-specific knockdown of a HTT transcript of Huntingtin (HTT) (e.g., WVE-003).

In some embodiments, the present disclosure provides compositions and methods for allele-specific knockdown of HTT transcripts, wherein allele-specific knockdown preferentially decreases the level, expression and/or activity of an allele(s) comprising Huntington's disease-associated mutation.

In some embodiments, a therapeutically effective amount of a HTT oligonucleotide, or a salt form thereof, or a HTT oligonucleotide composition is sufficient to mediate a clinically significant amount of allele-specific knockdown of a mutant HTT transcript in a subject.

In some embodiments, a therapeutically effective amount of a HTT oligonucleotide, or a salt form thereof, or a HTT oligonucleotide composition is low enough to prevent or reduce the occurrence and/or reduce the severity of at least about one adverse event mediated by administration of the oligonucleotide or oligonucleotide composition (e.g. when administered at a higher dose) to the subject. In some embodiments, a therapeutically effective amount of a HTT oligonucleotide, or a salt form thereof, or a HTT oligonucleotide composition is sufficient to mediate a clinically significant amount of allele-specific knockdown of a mutant HTT transcript in a subject, but is low enough to prevent or reduce the occurrence and/or reduce the severity of at least about one adverse event mediated by and/or associated with administration of the oligonucleotide or oligonucleotide composition (e.g. when administered at a higher dose) to the subject.

In some embodiments, an adverse event is an adverse effect. In some embodiments, an adverse event is mild, moderate, severe, or serious. In some embodiments, a serious adverse event is more severe than an adverse event categorized as severe, moderate or mild. In some embodiments, a serious adverse event is immediately life threatening, requires inpatient hospitalization or prolongation of existing hospitalization, results in persistent or significant disability/incapacity, or is a congenital anomaly/birth defect not present at screening. In some embodiments, an adverse event can be treated with hydrocortisone and/or acetaminophen.

In some embodiments, a severe adverse event is more severe than a moderate or mild adverse event. In some embodiments, a moderate adverse event is more severe than a mild adverse event. In some embodiments, an adverse event is: pyrexia, headache, vomiting, or tachycardia. In some embodiments, an adverse event is or is, is measured by, or is related to an increase in the inflammatory marker high-sensitivity C-reactive protein (hsCRP); an increase in complement factor Bb; or an increase in complement factor C3.

In some embodiments, a HTT oligonucleotide or a HTT oligonucleotide composition is an oligonucleotide or oligonucleotide composition which targets a HTT transcript and is capable of modulating allele-specific knockdown of a mutant HTT transcript of the target transcript. In some embodiments, a HTT oligonucleotide or a HTT oligonucleotide composition is useful for preparation of a medicament for treatment of Huntington's disease. In some embodiments, a HTT oligonucleotide or a HTT oligonucleotide composition is useful for treatment of Huntington's disease.

In some embodiments, a HTT oligonucleotide or a HTT oligonucleotide composition is useful for preparation of a medicament for treatment of Huntington's disease, wherein the oligonucleotide is WVE-003, and the medicament is administered in a dose equivalent to about 30, about 60, about 90, about 120, about 150 or about 168 mg WVE-003 free acid form.

In some embodiments, the present disclosure provides methods of use of a HTT oligonucleotide or HTT oligonucleotide composition. In some embodiments, a HTT oligonucleotide, or a salt form thereof, or a HTT oligonucleotide composition is capable of mediating an allele-specific decrease in the level, expression and/or activity of a mHTT transcript. In some embodiments, a HTT oligonucleotide, or a salt form thereof, or a HTT oligonucleotide composition is capable of mediating allele-specific knockdown of a mutant HTT transcript. In some embodiments, a HTT oligonucleotide, or a salt form thereof, or a HTT oligonucleotide composition is capable of mediating an allele-specific decrease in the level, expression and/or activity of mHTT transcript. In some embodiments, a HTT oligonucleotide, or a salt form thereof, or a HTT oligonucleotide composition is chirally controlled.

In some embodiments, a HTT oligonucleotide, or a salt form thereof, or a HTT oligonucleotide composition is or comprises WVE-003. In some embodiments, a HTT oligonucleotide, or a salt form thereof, or a HTT oligonucleotide composition is capable of mediating an allele-specific decrease in the level, expression and/or activity of mHTT and is chirally controlled. In some embodiments, a HTT oligonucleotide, or a salt form thereof, or a HTT oligonucleotide composition is capable of mediating an allele-specific decrease in the level, expression and/or activity of mHTT and is chirally controlled.

The present disclosure recognizes challenges of providing low toxicity HTT oligonucleotide compositions and methods of use thereof. In some embodiments, the present disclosure provides HTT oligonucleotide compositions and methods with reduced toxicity. In some embodiments, the present disclosure provides HTT oligonucleotide compositions and methods with reduced immune responses. In some embodiments, the present disclosure recognizes that various toxicities induced by HTT oligonucleotides are related to cytokine and/or complement activation. In some embodiments, the present disclosure provides HTT oligonucleotide compositions and methods with reduced or transient cytokine and/or complement activation. In some embodiments, the present disclosure provides HTT oligonucleotide compositions and methods with reduced complement activation via the alternative pathway. In some embodiments, the present disclosure provides HTT oligonucleotide compositions and methods with reduced complement activation via the classical pathway. In some embodiments, the present disclosure provides HTT oligonucleotide compositions and methods with reduced drug-induced vascular injury. In some embodiments, the present disclosure provides HTT oligonucleotide compositions and methods with reduced injection site inflammation. In some embodiments, reduced toxicity can be evaluated through one or more assays widely known to and practiced by a person having ordinary skill in the art, e.g., evaluation of levels of complete activation product, protein binding, etc.

### Chirally Controlled HTT Oligonucleotides and Compositions Thereof

Among other things, the present disclosure pertains to chirally controlled HTT oligonucleotides and oligonucleotides, e.g., WVE-003, and methods thereof.

Among other things, the present disclosure encompasses the recognition that, in contrast to chirally controlled HTT oligonucleotide compositions, stereorandom HTT oligonucleotide preparations contain a plurality of distinct chemical entities that differ from one another, e.g., in the stereochemical structure of individual backbone chiral centers within the HTT oligonucleotide chain. Without control of stereochemistry of backbone chiral centers, a stereorandom HTT oligonucleotide preparation (e.g., a random mixture of diastereoisomers) provides uncontrolled (or stereorandom) compositions comprising undetermined levels of HTT oligonucleotide stereoisomers. Even though these stereoisomers may have the same base sequence and/or chemical modifications, they are different chemical entities at least about due to their different backbone stereochemistry, and they can have different properties, e.g., activities, toxicities, distribution etc. Among other things, the present disclosure provides chirally controlled compositions that are or contain particular stereoisomers of HTT oligonucleotides of interest (e.g., WVE-003); in contrast to chirally uncontrolled compositions, chirally controlled compositions comprise controlled levels of particular stereoisomers of HTT oligonucleotides. In some embodiments, level of a particular stereoisomer, e.g., WVE-003, of a chirally controlled oligonucleotide composition is enriched as described herein (e.g., in some embodiments, each chiral internucleotidic linkage independently has a stereopurity of about 97%, 98%, 99% or more).

In some embodiments, a particular stereoisomer may be defined, for example, by its base sequence, its pattern of backbone linkages, its pattern of backbone chiral centers, and pattern of backbone phosphorus modifications, etc. As is understood in the art, in some embodiments, base sequence may refer solely to the sequence of bases and/or to the identity and/or modification status of nucleoside residues (e.g., of sugar and/or base components, relative to standard naturally occurring nucleotides such as adenine, cytosine, guanosine, thymine, and uracil) in a HTT oligonucleotide and/or to the hybridization character (i.e., the ability to hybridize with particular complementary residues) of such residues. In some embodiments, the present disclosure demonstrates that property improvements (e.g., improved activities, lower toxicities, etc.) achieved through inclusion and/or location of particular chiral structures within a HTT oligonucleotide can be comparable to, or even better than those achieved through use of chemical modifications, e.g., particular backbone linkages, residue modifications, etc. (e.g., through use of certain types of modified phosphates [e.g., phosphorothioate, substituted phosphorothioate, etc.], sugar modifications [e.g., 2′-modifications, etc.], and/or base modifications [e.g., methylation, etc.]). In some embodiments, the present disclosure demonstrates that chirally controlled HTT oligonucleotide compositions of HTT oligonucleotides (e.g., WVE-003, including various salt forms thereof) demonstrate unexpectedly high capability for mediating allele-specific knockdown of a mutant HTT transcript, and are useful for treatment and/or prevention of Huntington's disease.

In some embodiments, a composition of WVE-003 is a chirally controlled oligonucleotide composition capable of mediating an allele-specific decrease in the level, expression and/or activity of a mHTT transcript. In some embodiments, a chirally controlled WVE-003 composition is utilized to target human Huntingtin pre-messenger ribonucleic acid (mRNA) in order to induce allele-specific knockdown of a mutant HTT transcript and Huntingtin protein restoration in subjects with Huntington's disease (HTT). WVE-003 comprises a base sequence that can be antisense to and hybridize by complementary base pairing to a HTT transcript.

Applicant has developed technologies that enable the synthesis of linkage modified nucleic acid therapeutics in which stereochemistry at each chiral linkage phosphorus position is precisely controlled. This degree of control enables rational design and synthesis of optimized stereopure, oligonucleotides with improved pharmacological and toxicological properties. WVE-003 are developed and manufactured using certain such technologies.

In some embodiments, WVE-003 is utilized to target human Huntingtin (HTT) pre-messenger ribonucleic acid (mRNA) in order to induce allele-specific knockdown of a mutant HTT transcript in subjects with Huntington's disease (HTT).

In some embodiments, the present disclosure provides oligonucleotides, oligonucleotide compositions, and methods of use thereof for mediating an allele-specific decrease in the level, expression and/or activity of mHTT in HTT (e.g., of mouse, human, etc.).

In some embodiments, an oligonucleotide composition comprises WVE-003. In some embodiments, an oligonucleotide composition is a chirally controlled oligonucleotide composition of WVE-003. In some embodiments, such a composition is a pharmaceutical composition of WVE-003.

As appreciated by those skilled in the art, an oligonucleotide, e.g., WVE-003, may be administered in one or more forms (e.g., acid forms, various salt forms, etc.). In some embodiments, a form is an acid form. In some embodiments, a form is a salt form. In some embodiments, a form is a sodium salt form. In some embodiments, an oligonucleotide, e.g., WVE-003, is administered as a salt form, e.g., a sodium salt form, optionally in a solution. In some embodiments, a composition, e.g., a pharmaceutical composition, a chirally controlled oligonucleotide composition, etc., comprises one or more forms of WVE-003. As appreciated by those skilled in the art, in some embodiments, an amount of an oligonucleotide, e.g., an amount of an oligonucleotide being administered, is the corresponding amount in a particularly form (e.g., an acid form) of all forms, e.g., all forms being administered (e.g., one or more salt forms (e.g., sodium salt form). In some embodiments, multiple forms of an oligonucleotide may exist in a composition.

In some embodiments, compositions comprising WVE-003 are each a chirally pure or chirally controlled oligonucleotide composition of WVE-003.

In some embodiments, chirally controlled oligonucleotide compositions are typically prepared through chirally controlled oligonucleotide preparation to stereoselectively form one or more chiral internucleotidic linkages (e.g., using chiral auxiliaries as exemplified in the present disclosure, compared to non-chirally controlled (stereorandom, non-stereoselective, racemic) oligonucleotide synthesis such as traditional phosphoramidite-based oligonucleotide synthesis using no chiral auxiliaries or chiral catalysts to purposefully control stereoselectivity). A chirally controlled oligonucleotide composition of an oligonucleotide is enriched, relative to a substantially racemic preparation of oligonucleotides having the same base sequence and the same modifications, for the particular oligonucleotide (e.g., for chirally controlled oligonucleotide compositions of WVE-003, enriched for WVE-003). As one having ordinary skill in the art readily appreciates, such enrichment can be characterized in that compared to a substantially racemic preparation, at each chirally controlled internucleotidic linkage, a higher level of the linkage phosphorus has the desired configuration. In some embodiments, each chirally controlled internucleotidic linkage independently has a diastereopurity of at least 80%, 85%, 90%, 91%, 92%, 93%, 94%, 95%, 96%, 97%, 98%, or 99% with respect to its chiral linkage phosphorus. In some embodiments, a diastereopurity is at least 95%. In some embodiments, a diastereopurity is at least 96%. In some embodiments, a diastereopurity is at least 97%. In some embodiments, a diastereopurity is at least 98%. In some embodiments, a diastereopurity is about 99% or more.

As appreciated by those skilled in the art, a preparation typically comprises an oligonucleotide, e.g., WVE-003, and one or more other oligonucleotides and/or other impurities, as manufacturing processes rarely can reach 100% selectivity and/or purity (including but not limited to diastereomeric purity). Those skilled in the art appreciate that levels of such other oligonucleotides and/or impurities can and are properly controlled.

In some embodiments, a chirally controlled oligonucleotide composition can consist essentially of a desired oligonucleotide (e.g., WVE-003), e.g., the other oligonucleotides being impurities from manufacturing of that oligonucleotide. In some embodiments, impurities include oligonucleotides which are similar but different than the desired oligonucleotide (e.g., one or more internucleotidic linkage in the impurity may have an undesired configuration; and/or the impurity can be shorter or longer than the desired oligonucleotide). In some embodiments, purity levels of various preparations of WVE-003 are described herein. In some embodiments, characteristics of various impurities are as described herein. Without wishing to be bound by any particular theory, the present disclosure note that it is possible that one or more impurities may be similar enough to the desired oligonucleotide to mediate a desired activity (e.g., allele-specific knockdown of mHTT).

As described herein, in some embodiments, a purity (e.g., a purity level, for example of WVE-003) is about or at least about 75%. In some embodiments, a purity is about or at least about 80%. In some embodiments, a purity is about or at least about 81%. In some embodiments, a purity is about or at least about 82%. In some embodiments, a purity is about or at least about 83%. In some embodiments, a purity is about or at least about 84%. In some embodiments, a purity is about or at least about 85%. In some embodiments, a purity is about or at least about 86%. In some embodiments, a purity is about or at least about 87%. In some embodiments, a purity is about or at least about 88%. In some embodiments, a purity is about or at least about 89%. In some embodiments, a purity is about or at least about 90%. In some embodiments, a purity is about or at least about 91%. In some embodiments, a purity is about or at least about 92%. In some embodiments, a purity is about or at least about 93%. In some embodiments, a purity is about or at least about 94%. In some embodiments, a purity is about or at least about 95%. In some embodiments, a purity is about or at least about 96%. In some embodiments, a purity is about or at least about 97%. In some embodiments, a purity is about or at least about 98%. In some embodiments, a purity is about or at least about 99%.

In some embodiments, purity of WVE-003 in a preparation, composition, medicament, etc., can be determined using various suitable methods as described herein. In some embodiments, purity is assessed using chromatography with UV detection, e.g., UPLC-UV as described in, and purity is measured as peak area % at a given wavelength, e.g., 260 nm. In some embodiments, a purity is assessed as described in Protocol A. In some embodiments, a purity is assessed as described in Protocol B. In some embodiments, a purity is assessed by dimer modeling.

### Manufacturing

In some embodiments, the present disclosure provides technologies for manufacturing an oligonucleotide, e.g., WVE-003, that are particularly useful for stereoselective large scales preparations. In some embodiments, a preparation of WVE-003, e.g. WVE-003 drug substance or drug product, is a solid. In some embodiments, it is in a white to off-white powder. In some embodiments, solubility of prepared WVE-003 in water was determined to be at least 79.20 mg/mL as determined by UV spectrophotometry (e.g., at 260 nm). In some embodiments, pH of WVE-003 preparations in purified water is from 6.0-8.0. Unless noted otherwise, extinction coefficient of 181181 M⁻¹cm⁻¹ is utilized herein, e.g., to calculate WVE-003 concentrations/amounts from UV at 260 nm.

In some embodiments, an oligonucleotide, e.g., WVE-003, is chemically synthesized using commercially available synthesizers in compliance with appropriate cGMP regulations.

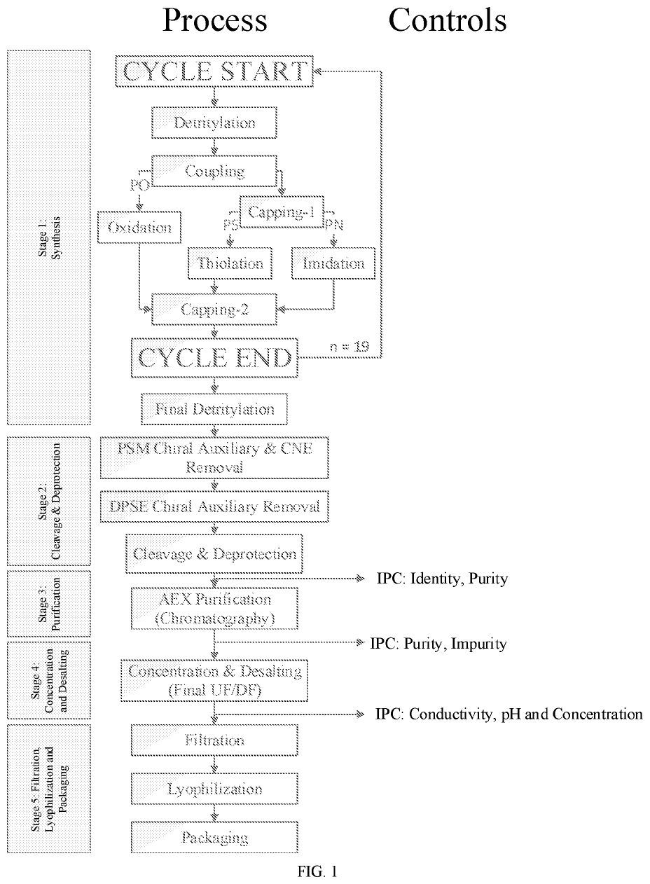

Manufacture of WVE-003, e.g., drug substance, is a multi-step process that includes solid-phase oligonucleotide synthesis, cleavage of the crude protected oligonucleotide from the solid support, removal of protecting groups (deprotection), preparative anion exchange (AEX) chromatographic purification, concentration and desalting, filtration, lyophilization and packaging. A flow diagram depicting the WVE-003 drug substance manufacturing process is shown in FIG. 1.

In some embodiments, stereochemistry of an oligonucleotide, e.g., WVE-003, is established through control of the starting materials for synthesis as well as the synthetic process. For example, use of phosphoramidites prepared from the chiral auxiliaries (L)-DPSE, (D)-DPSE and (L)-PSM during the coupling step ensures that the intended stereodefined Sp phosphorothioate diester, Rp phosphorothioate diester and Rp N-(1,3-dimethylimidazolidin-2-ylidene) phosphoramidate diester linkages, respectively, are obtained.

In some embodiments, the present disclosure provides methods for manufacturing various drug products as described herein.

In some embodiments, the present disclosure provides products (e.g., crude/purified oligonucleotides from stereoselective preparation, crude compositions, purified compositions, formulated compositions, pharmaceutical compositions, drug substances, drug products, etc.) by provided processes. In some embodiments, provided products are of certain purities as described herein. In some embodiments, provided products are suitable for therapeutic uses as described herein. In some embodiments, provided products achieve one or more properties and/or activities as described herein.

Certain processes useful for manufacturing WVE-003, and various compositions and products thereof, are described below as examples. Several batches of WVE-003 drug substance and/or drug products were manufactured.

As shown herein, preparation of WVE-003 comprises multiple cycles (e.g., as described below). In some embodiments, each cycle to introduce a stereodefined phosphorothioate or stereodefined phosphoramidate consists of 5′-detritylation, coupling, capping of exposed chiral auxiliary secondary amine (Capping-1), thiolation or imidation, respectively, and capping of unreacted 5′-hydroxyl groups (Capping-2), each cycle to introduce a phosphodiester consists of 5′-detritylation, coupling, oxidation, and Capping-2, and each coupling reaction is carried out by activation of the appropriate phosphoramidite and reaction with the free 5′-hydroxyl group of a support-immobilized protected nucleoside or oligonucleotide. In some embodiments, after the appropriate number of cycles and final detritylation, the (L)-PSM chiral auxiliary and cyanoethyl phosphate protecting groups are removed from the crude oligonucleotide by on-column treatment with diethylamine (DEA) in acetonitrile (ACN), and the (L) and (D) DPSE chiral auxiliaries are removed by treatment with TEA·HF (triethylamine hydrofluoride) solution. The crude oligonucleotide is then cleaved from the solid support by treatment with ammonium hydroxide in an appropriately sized pressure-rated vessel. This reaction also effects the global deprotection of exocyclic amino groups (acetyl, benzoyl, and isobutyryl). In some embodiments, the resulting crude oligonucleotide is purified using anion exchange (AEX) chromatography, and the purified oligonucleotide is concentrated and desalted by tangential flow filtration, followed by filtration, lyophilization and packaging to yield the WVE-003 drug substance. Certain steps to manufacture WVE-003 drug substance are described in further detail below as examples. Certain key functional groups are described below.

| X | Yⁿ | R | Br |
|---|---|---|---|
| S | H | (L)-DPSE-Ac | A^{Bz} |
| S | H | (L)-DPSE-Ac | C^{Ac} |
| S | H | (L)-DPSE-Ac | G^{iBu} |
| S | H | (D)-DPSE-Ac | G^{iBu} |
| S | H | (L)-DPSE-Ac | T |
| S | H | (D)-DPSE-Ac | T |
| S | OMe | (L)-DPSE-Ac | A^{Bz} |
| S | OMe | (L)-DPSE-Ac | G^{iBu} |
| S | MOE | (L)-DPSE-Ac | 5MeC^{Bz} |
| N | MOE | (L)-PSM-Ac | 5MeC^{Bz} |
| N | MOE | (L)-PSM-Ac | G^{iBu} |
| N | OMe | (L)-PSM-Ac | G^{iBu} |
| N | OMe | (L)-PSM-Ac | U |
| O | MOE | CNE | A^{Bz} |
| O | OMe | CNE | U |

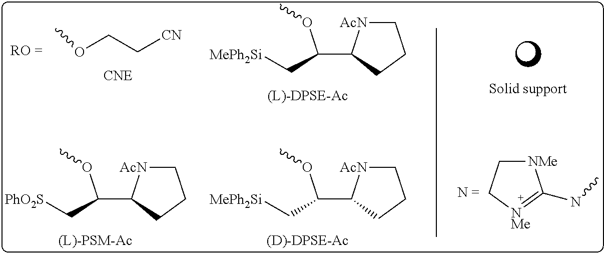

### Cycle for Phosphorothioate Assembly

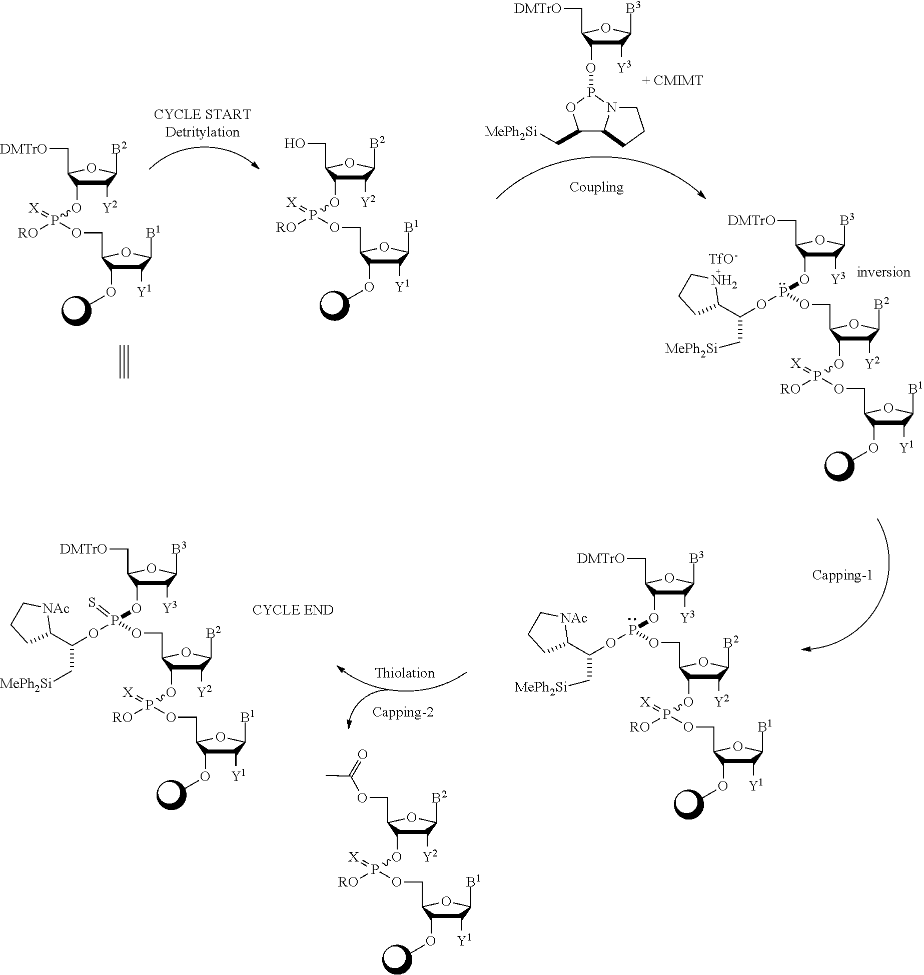

### Cycle for Phosphorimidate Assembly

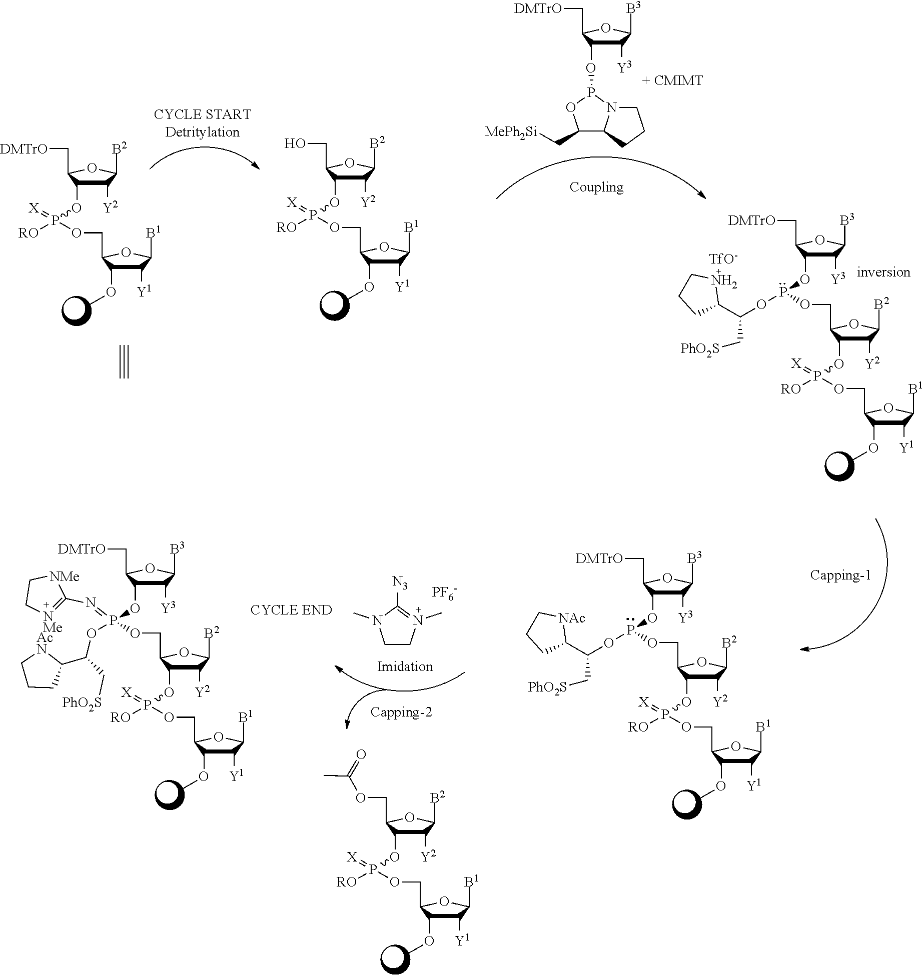

### Cycle for Phosphodiester Assembly

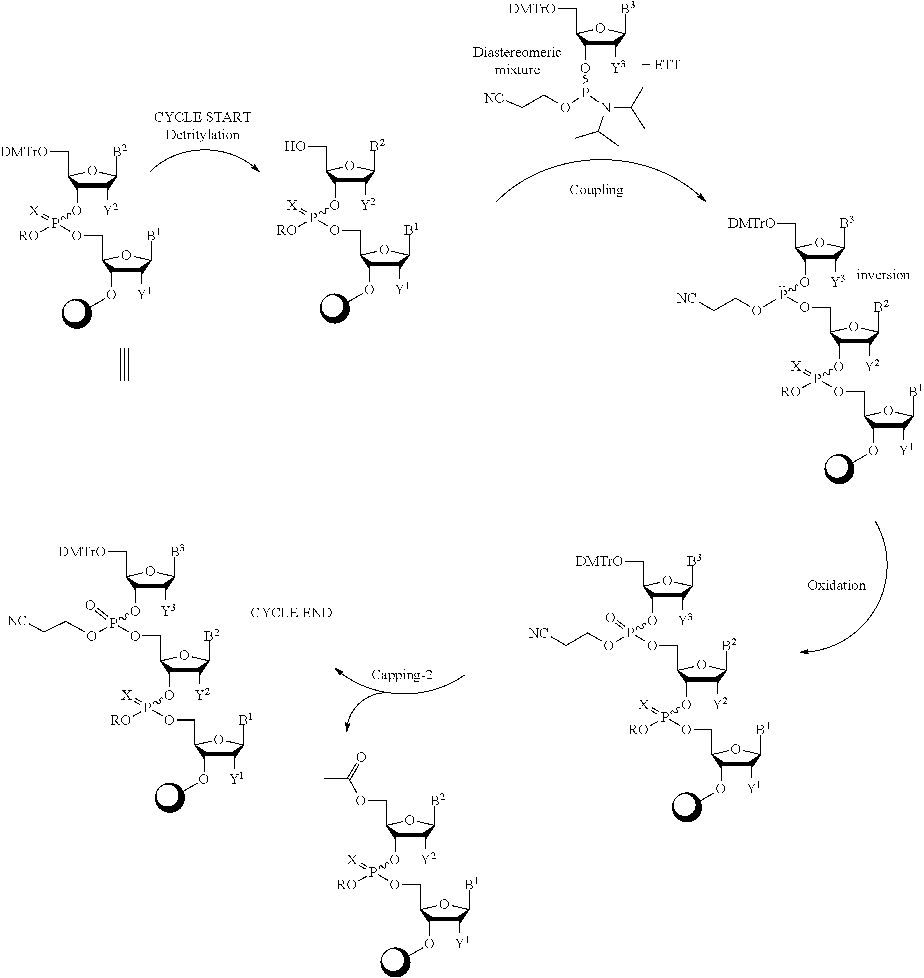

### Stage 1—Synthesis

Synthesis of the oligonucleotide is carried out on Controlled Pore Glass (CPG) solid support functionalized with 5′-ODMTr-2′-MOE-T, on an automated oligonucleotide synthesizer. All reactions take place on the solid support packed in a column.

### Detritylation

To initiate the synthesis, the CPG-5′-ODMTr-2′-MOE-T solid support is subjected to acid-catalyzed removal of the DMTr protecting group from the 5′-hydroxyl by treatment with 3% dichloroacetic acid (DCA) in toluene. Complete DMTr removal is ensured by inline UV monitoring based on a watch command in the synthesis program. The DMTr removal is performed in the same way at the beginning of each synthesis cycle and after the final cycle. In every case, following detritylation, the support-bound material is washed with acetonitrile in preparation for the next step of the synthesis.

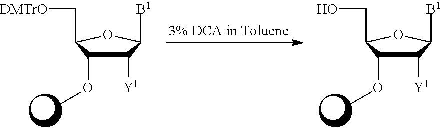

### Coupling

Elongation of the growing oligonucleotide chain is achieved by reaction of the 5′-hydroxyl group of the support-bound oligonucleotide with an excess of a solution of the protected phosphoramidite, in the presence of an activator, either 5-(ethylthio)-1H-tetrazole (ETT) or 1-cyanomethyl imidazolium triflate salt (CMIMT) dissolved in acetonitrile. The phosphoramidites are dissolved in acetonitrile or isobutyronitrile or an 80:20 v/v acetonitrile:isobutyronitrile mixture. Among other things, the present disclosure recognizes that ethyl acetate can be utilized for preparing phosphoramidite solutions for oligonucleotide preparations, e.g., WVE-003 preparations. In some embodiments, ethyl acetate can be utilized to replace isobutyronitrile. In some embodiments, utilization of ethyl acetate reduces manufacturing cost and/or simplifies operations. In some embodiments, phosphoramidites are dissolved in ethyl acetate or a mixture thereof. In some embodiments, a solvent is ethyl acetate. In some embodiments, a solvent is a mixture of ethyl acetate and acetonitrile (e.g., about 50:50 v/v ethyl acetate:acetonitrile, about 20:80 v/v ethyl acetate:acetonitrile, etc.). Among other things, the present disclosure recognizes that propylene carbonate can be utilized for preparing phosphoramidite solutions for oligonucleotide preparations, e.g., WVE-003 preparations. In some embodiments, propylene carbonate can be utilized to replace isobutyronitrile. In some embodiments, utilization of propylene carbonate reduces manufacturing cost and/or simplifies operations. In some embodiments, phosphoramidites are dissolved in propylene carbonate or a mixture thereof. In some embodiments, a solvent is propylene carbonate. In some embodiments, a solvent is a mixture of propylene carbonate and acetonitrile (e.g., about 50:50 v/v propylene carbonate:acetonitrile). In some embodiments, phosphoramidites are dissolved in acetonitrile, ethyl acetate, an ethyl acetate and acetonitrile mixture (e.g., about 20:80 v/v ethyl acetate:acetonitrile; about 50:50 v/v ethyl acetate:acetonitrile; etc.), or a propylene carbonate and acetonitrile mixture (e.g., about 50:50 v/v propylene carbonate:acetonitrile). The phosphoramidite required in each coupling step is determined by the oligonucleotide sequence. For introduction of phosphodiester linkage, phosphoramidites and ETT activator are used. For introduction of a stereodefined phosphorothioate or phosphoramidate linkage, (L)- or (D)-DPSE or (L)-PSM derived phosphoramidite respectively, and the CMIMT activator are used. In either case, the phosphoramidite/activator solutions are mixed inline, pushed onto the synthesis column, then recirculated through the column for appropriate amount of time. Subsequently, excess reagents are removed by flushing the synthesis column with acetonitrile.

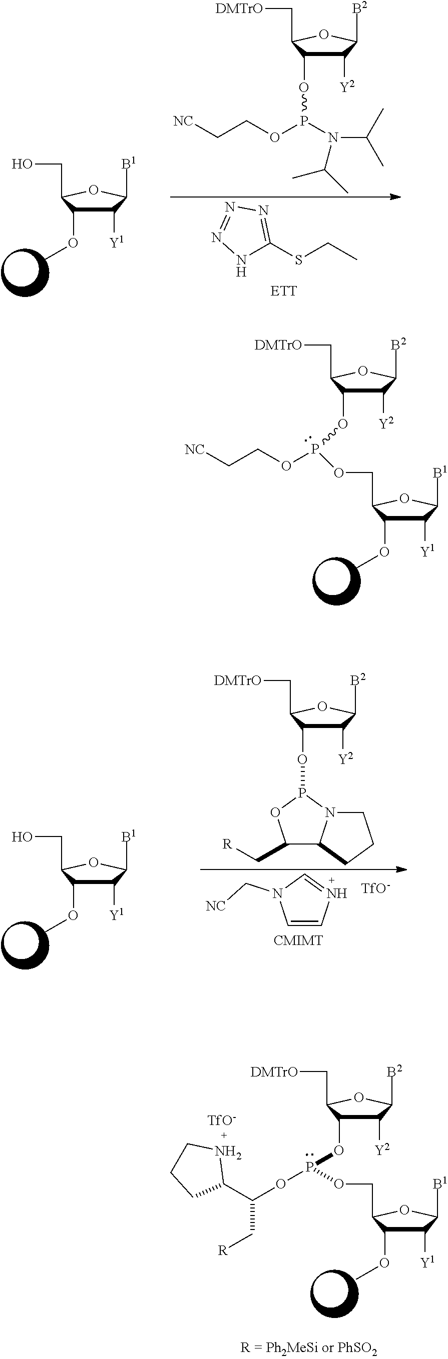

### Capping-1

The Capping 1 step is performed to protect the secondary amine which results from the oxazaphospholidine ring opening on the auxiliary group during the coupling step rendering it unreactive for the remainder of the synthesis. The secondary amine is capped by flowing capping reagent B ([Cap B]: acetic anhydride/2,6-lutidine/acetonitrile (20:30:50, v:v:v) through the synthesis column.

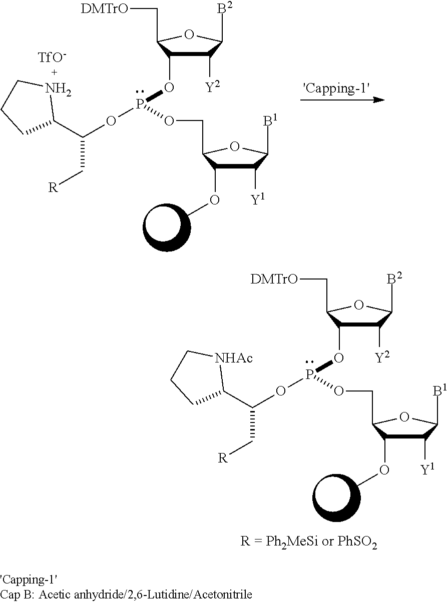

### Oxidation, Thiolation, Imidation

The newly created P(III) phosphite triester linkage is then treated in one of three ways: Oxidation by treatment with iodine in pyridine:water (90:10, v:v), resulting in generation of a P(V) phosphodiester linkage.

Thiolation by treatment with xanthane hydride (XH) in pyridine:acetonitrile resulting in the generation of a stereodefined P(V) phosphorothioate triester linkage.

Imidation by treatment with 2-azido-1,3-dimethylimidazolinium hexafluorophosphate (ADIH) in acetonitrile resulting in the generation of a stereodefined P(V) phosphorimidate triester linkage. In each case, after the step, the excess reagent is flushed from the synthesis column with acetonitrile.

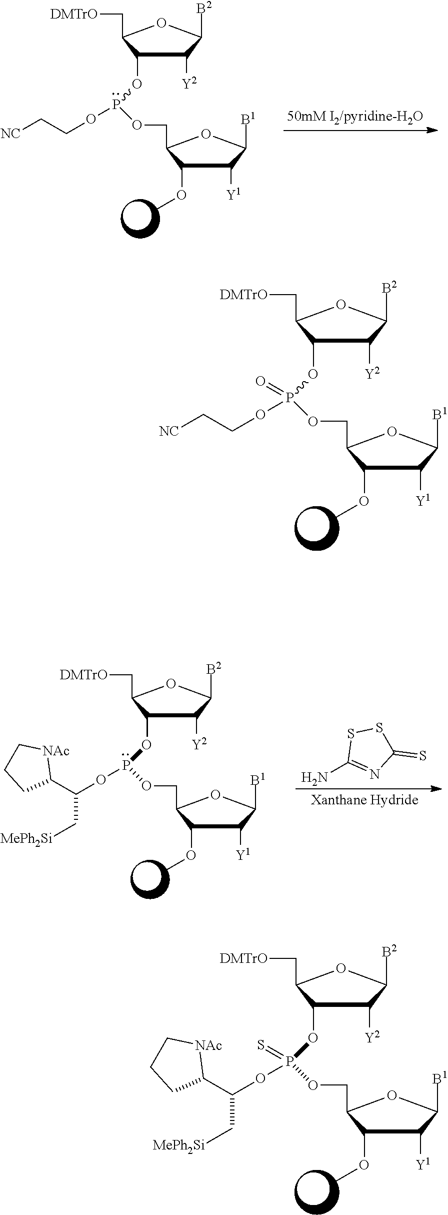

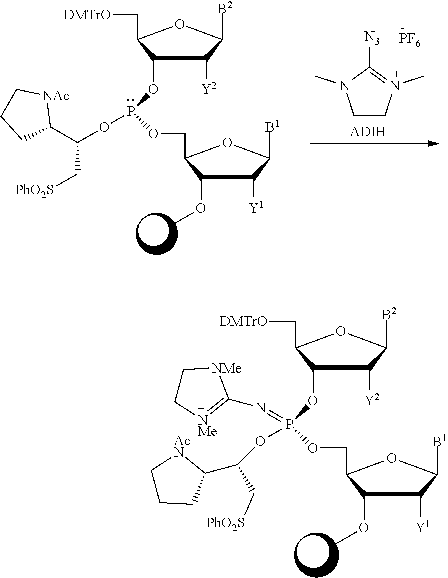

### Capping-2

Although the coupling reaction proceeds in very high yield it is not quantitative. A small proportion of the 5′-hydroxy groups, available in any given cycle fails to couple with the activated phosphoramidite. To prevent reaction during subsequent cycles, these sites are blocked by performing the capping-2 step, using capping reagents (1:1, v:v) (Capping Reagent A [Cap A]: N-methylimidazole/acetonitrile (20:80, v:v) and Cap B). As a result, 5′-O-acetylated (‘capped’) support-bound oligonucleotide sequences are formed. The excess capping reagents are washed from the column with acetonitrile.

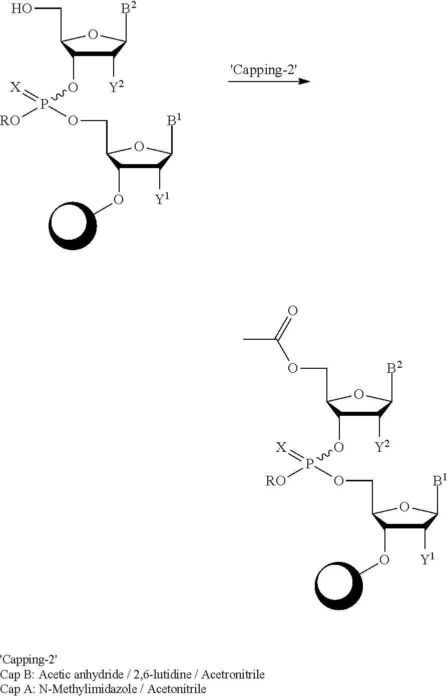

### Oligonucleotide Assembly and Final Step

Independent iteration of the cycles, using the appropriate protected phosphoramidites allows assembly of the entire protected sequence with the DMTr protecting group present at the 5-terminal position. After addition of the last nucleotide in the sequence, the 5-terminal DMTr group is removed during a final detritylation step.

### Stage 2—Cleavage and Deprotection

Removal of phosphate protecting groups, (L)-PSM and (L)- and (D)-DPSE chiral auxiliaries from stereodefined phosphorimidate triester and phosphorothioate triester, cleavage of the crude oligonucleotide from the solid support, and removal of exocyclic nucleobase protecting groups is performed in a three-step process.

Step 1: (L)-PSM Chiral Auxiliary and Cyanoethyl Removal with Diethylamine Treatment

The crude oligonucleotide on the solid support is treated with a solution of diethylamine in ACN which simultaneously removes cyanoethyl protecting groups from the phosphate triester linkages, and (L)-PSM chiral auxiliary from the phosphorimidate triester to generate phosphodiester and phosphoramidate diester linkages, respectively.

(L)-PSM chiral auxiliary and Cyanoethyl Removal with Diethylamine Treatment:

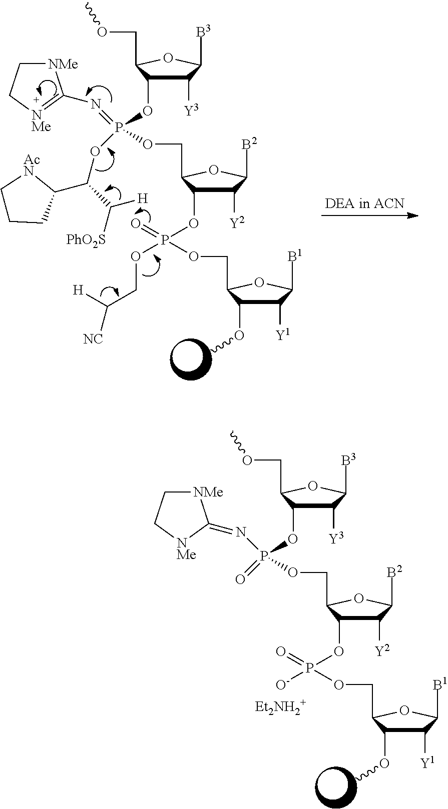

Step 2: (L)- and (D)-DPSE Chiral Auxiliary Removal with Fluoride Treatment

The crude oligonucleotide on the solid support is treated with a temperature-controlled solution of TEA·HF in a mixture of dimethyl sulfoxide (DMSO) and H₂O to effect removal of the chiral auxiliary. This process converts the stereodefined phosphorothioate triester to a stereodefined phosphorothioate diester.

(L)- and (D)-DPSE chiral auxiliary removal with Fluoride Treatment:

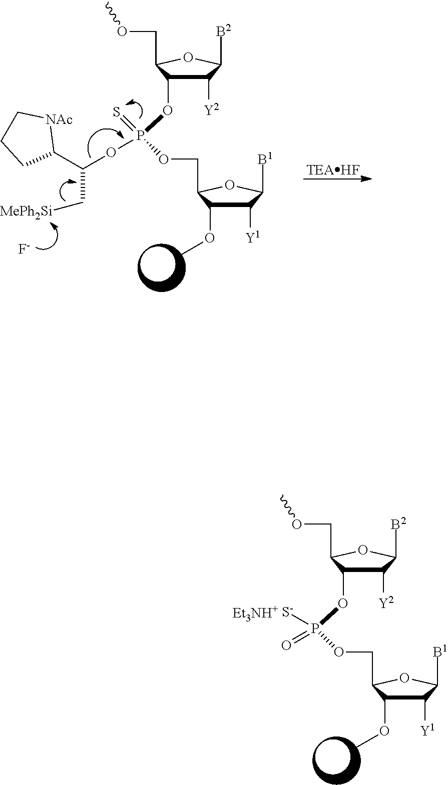

### Step 3: Cleavage and Deprotection

The crude oligonucleotide is then cleaved from the solid support by treatment with ammonium hydroxide in an appropriately sized pressure-rated vessel. This reaction effects the global deprotection of exocyclic amino groups (acetyl, benzoyl, and isobutyryl).

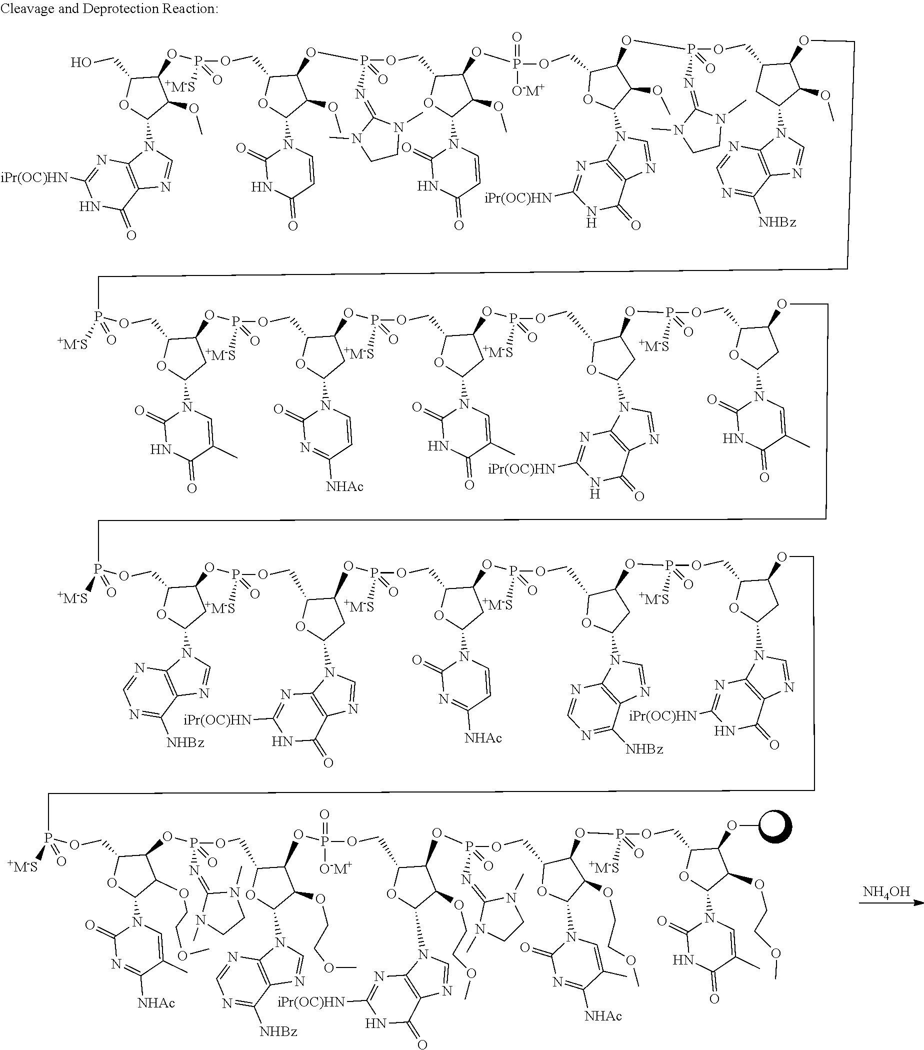

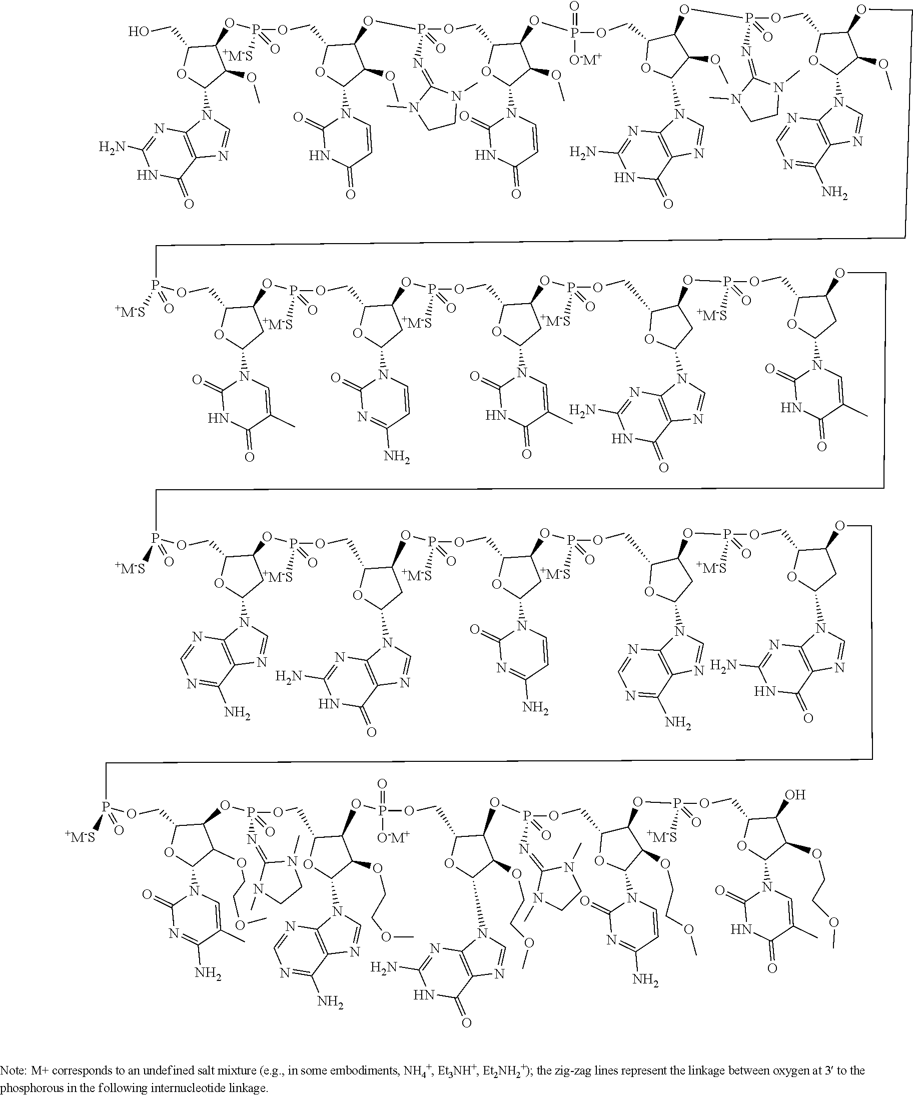

### Stage 3: Purification by Anion Exchange Chromatography

Purification of the crude oligonucleotide solution is accomplished by AEX chromatography. A solution of crude oligonucleotide is loaded onto the purification column packed with TSK-GEL Super Q-5PW media.

The purification is performed using sodium hydroxide buffered eluents. A sodium chloride gradient is used to elute the oligonucleotide from the column. The elution profile is monitored by ultraviolet (UV) spectrophotometry. Fractions are collected and neutralized with a sodium phosphate buffer. Mock pools are evaluated by IP-RP-UPLC. The pool containing oligonucleotide with the desired purity is subjected to the next step in the process.

### Stage 4: Concentration and Desalting (Final UF/DF)

The selected fraction pool is then concentrated and diafiltered against purified water to remove the purification buffer by tangential flow filtration (TFF) using regenerated cellulose membrane cassettes. The ultrafiltration/diafiltration (UF/DF) process proceeds as follows, the selected pool of fractions is pH neutralized with hydrochloric acid or sodium hydroxide and then concentrated. The concentrated oligonucleotide is diafiltered against purified water, further concentrated and collected. The system is flushed with purified water to maximize yield combining the concentrated desalted oligonucleotide with the rinses to give the final oligonucleotide solution.

### Stage 5: Filtration, Lyophilization and Packaging

The oligonucleotide solution is filtered through a 0.2-micron filter and then placed in freeze drying tray(s) for lyophilization. After lyophilization, the final drug substance is isolated as a solid powder, which is packaged in sterile high-density polyethylene (HDPE) bottles, each of which is labeled and sealed in a Mylar foil pouch and stored at −20° C.

### Control of Materials

### Starting Materials Used in the Manufacture of the Drug Substance

Useful raw materials (solvents, reagents and auxiliary materials) used in the manufacture of WVE-003, e.g., drug substance are listed below. In some embodiments, quality of all raw materials is controlled by material specifications and/or certificates of analysis.

The starting materials used in the preparation of WVE-003 drug substance include 15 phosphoramidites, the Controlled Pore Glass (CPG) solid support and the 2-azido-1,3-dimethylimidazolinium hexafluorophosphate (ADIH). Reactive exocyclic groups on nucleobases are typically appropriately protected to render them unreactive during oligonucleotide synthesis, and the 5′-hydroxy functionality is protected as a 4,4′-dimethoxytrityl ether (DMTr). In some embodiments, WVE-003 starting materials are released based on a set of material specifications. In some embodiments, phosphoramidites have purity levels of about 85% or more, in many cases about 90% or more, in many cases about 95% or about 98% or more (RP-HPLC at about 260 nm (area %) and/or ³¹P NMR by integration), about 97% or more P(III) purity (by P NMR integration), and moisture content of less than about 0.4% or in many cases less than (0.2) (w/w). In some embodiments, Controlled Pore Glass 5′-ODMTr-2′-OMOE T solid support is a white to off white powder, 120-200 mesh particle size (analytical sieving), 540-600 Å pore diameter (mercury intrusion), 0.20-024 g/cc density (tap density), ≥0.8 cc/g pore volume (mercury intrusion), ≥75 m²/g surface area (mercury intrusion) and 70-80 mol/g in DM1 ligand assay (spectrophotometric at 498 nm). In some embodiments, 2-Azido-1,3-dimethylimidazolinium hexafluorophosphate has a purity of about 98.0% or more (HPLC) and nitrogen content of about 23.70-24.80 (elemental analysis).

Certain Solvents, Reagents and Auxiliary Materials Used in the Manufacture of WVE-003, e.g., Drug Substance

| | Quality | |
|---|---|---|
| Material | Standard | Stop |
| Acetonitrile (ACN) | Supplier CoA | Synthesis (Solvent) |
| Capping reagent A: | Supplier CoA | Synthesis (Capping-2) |
| N-Methylimidazole (NMI) in acetonitrile (20% | | |
| NMI) | | |
| Capping reagent B: | Supplier CoA | Synthesis (Capping 1 and 2) |
| Acetic anhydride:2,6-Lutidine:Acetonitrile | | |
| (20:30:50) | | |
| Dichloroacetic acid in toluene (3%) | Supplier CoA | Synthesis (Detritylation) |
| 5-(Ethylthio)-1H-tetrazole (ETT) in acetonitrile | Supplier CoA | Synthesis (Coupling Activator) |
| 1-(Cyanomethyl)-1H-imidazole triflate (CMIMT) | Supplier CoA | Synthesis (Coupling Activator) |
| Xanthane Hydride (XH) | Supplier CoA | Synthesis (Thiolation) |
| Iodine (0.05M) in Pyridine:water (90:10) | Supplier CoA | Synthesis (Oxidation) |
| Pyridine | ACS | Synthesis (Solvent) |
| Isobutyronitrile (IBN) | Supplier CoA | Synthesis (Solvent) |
| Molecular Sieve 3A, 8 to 12 Mesh | Supplier CoA | Synthesis (Adsorbent) |
| Nitrogen | NF | Synthesis (Inert gas) |
| Dichloromethane | Supplier CoA | Amidite lines Clean-in-place |
| Ammonium hydroxide (28%) | ACS | Cleavage, Deprotection |
| Triethylamine (TEA) | Supplier CoA | Cleavage, Deprotection |
| Triethylamine•trihydrofluoride (TEA•3HF), 97% | Supplier CoA | Cleavage, Deprotection |
| Dimethyl Sulfoxide (DMSO) | ACS | Cleavage, Deprotection |
| Diethylamine (DEA) | Supplier CoA | Cleavage, Deprotection |
| Water for Injection (WFI) | USP | Solvent |
| Sodium chloride | USP/NF | Purification (Buffer salt) |
| Sodium hydroxide, 10N | Supplier CoA | Purification, Neutralization, CIP |
| Hydrochloric acid, 0.1N | Supplier CoA | PH Neutralization |
| Tosoh TSK-Gel Super Q-5PW | Supplier CoA | Purification media |

Abbreviations: CoA = Certificate of Analysis;
NF = National Formulary;
CIP = clean in place;
USP = United States Pharmacopoeia;
ACS = American Chemical Society

Certain starting Materials Used in the Manufacture of WVE-003 Drug Substance

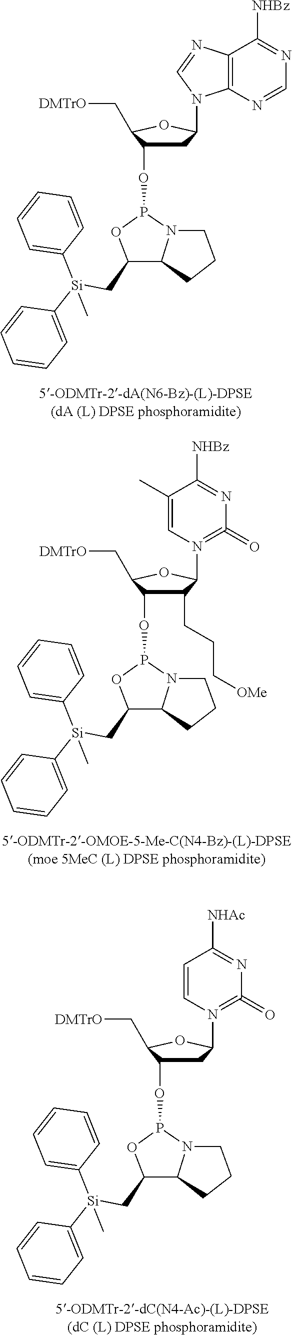

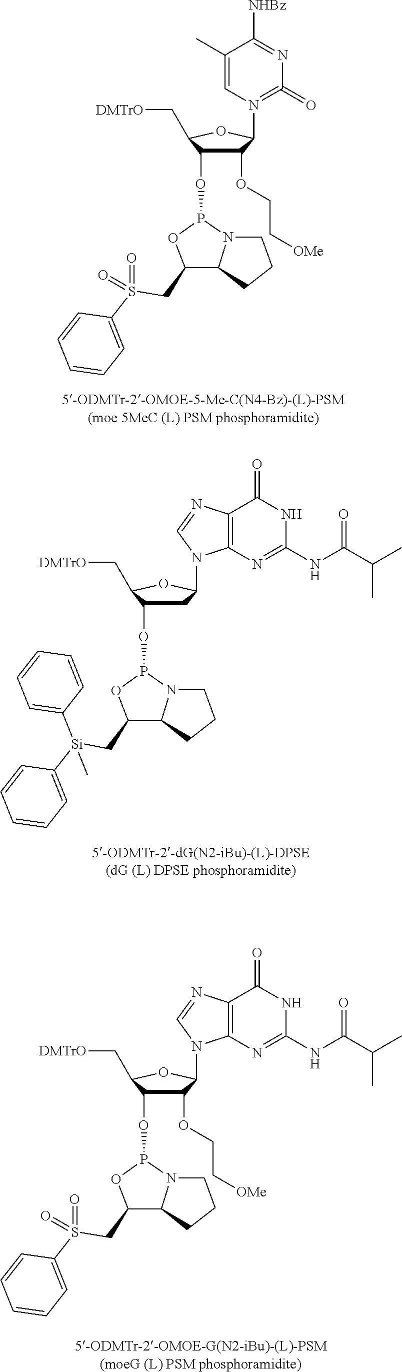

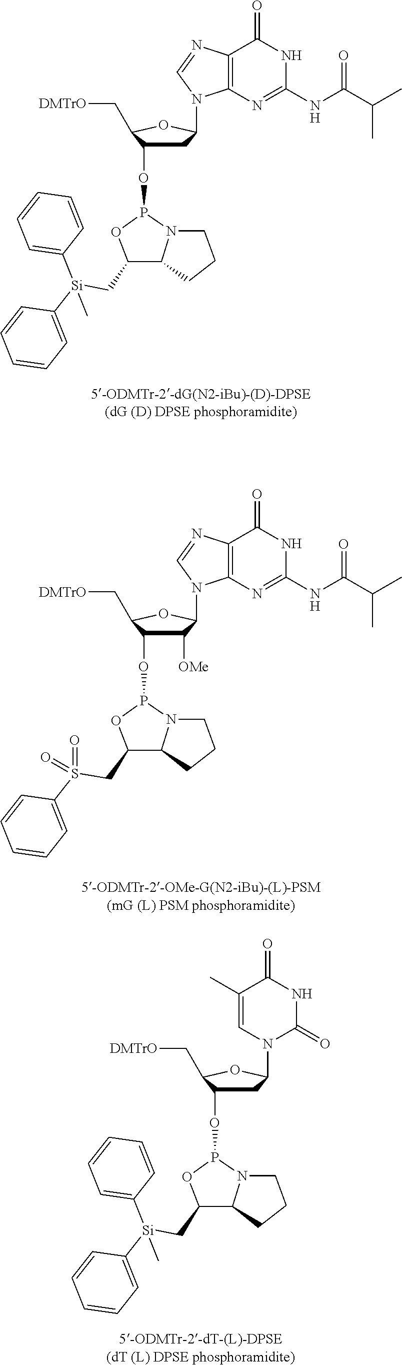

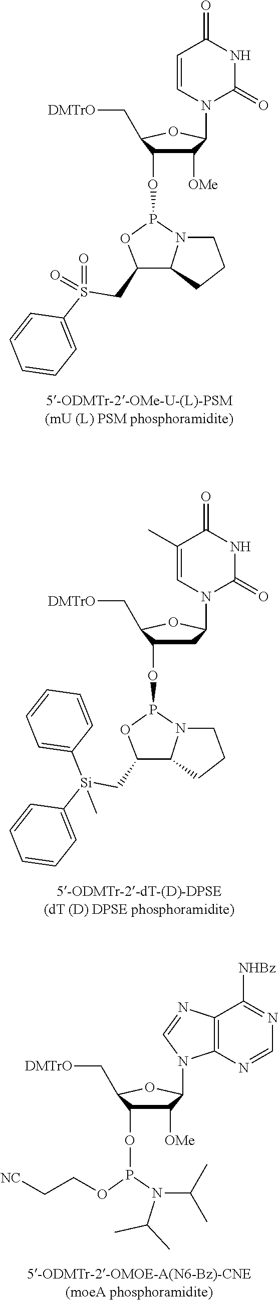

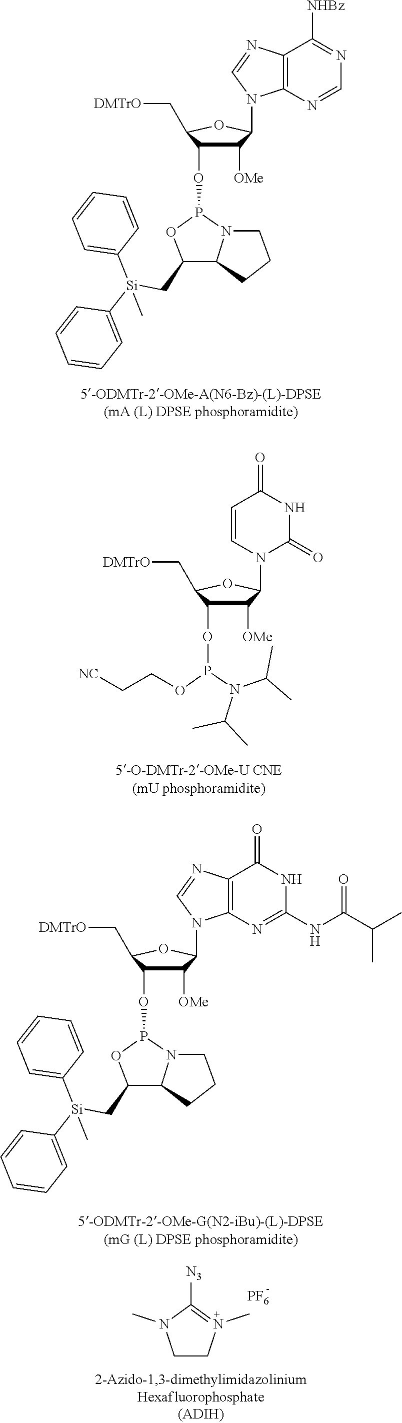

### Certain Solvents Useful for Preparing Phosphoramidite Solutions for Oligonucleotide Preparations

| Material | Solvent |
|---|---|
| dA(bz) (L) DPSE phosphoramidite | acetonitrile |
| dG(ibu) (L) DPSE phosphoramidite | acetonitrile |
| dG(ibu) (D) DPSE phosphoramidite | acetonitrile |
| dC(ac) (L) DPSE phosphoramidite | ethyl acetate |
| dT (L) DPSE phosphoramidite | acetonitrile |
| dT (D) DPSE phosphoramidite | acetonitrile |
| mA(bz) (L) DPSE phosphoramidite | 50% ethyl acetate/acetonitrile mixture (% v) |
| mG(ibu) (L) DPSE phosphoramidite | 20% ethyl acetate/acetonitrile mixture (% v) |
| mG(ibu) (L) PSM phosphoramidite | 50% propylene carbonate/acetonitrile mixture |
| | (% v) |
| mU (L) PSM phosphoramidite | acetonitrile |
| mU phosphoramidite (2′-cyanoethyl and N,N- | 20% ethyl acetate/acetonitrile mixture (% v) |
| diisopropyl) | |
| Aeo(bz) phosphoramidite (2′-cyanoethyl and N,N- | acetonitrile |
| diisopropyl) | |
| Geo(ibu) (L) PSM phosphoramidite | acetonitrile |
| m5Ceo (bz) (L) DPSE phosphoramidite | 20% ethyl acetate/acetonitrile mixture (% v) |
| m5Ceo (bz) (L) PSM phosphoramidite | acetonitrile |

Stereochemistry in WVE-003 can be established through control of the starting materials for synthesis as well as the synthetic process. Stereodefined phosphoramidite starting materials for manufacture of WVE-003 are prepared from appropriately protected nucleosides and a chiral auxiliary, (L)- and (D)-DPSE and (L)-PSM:

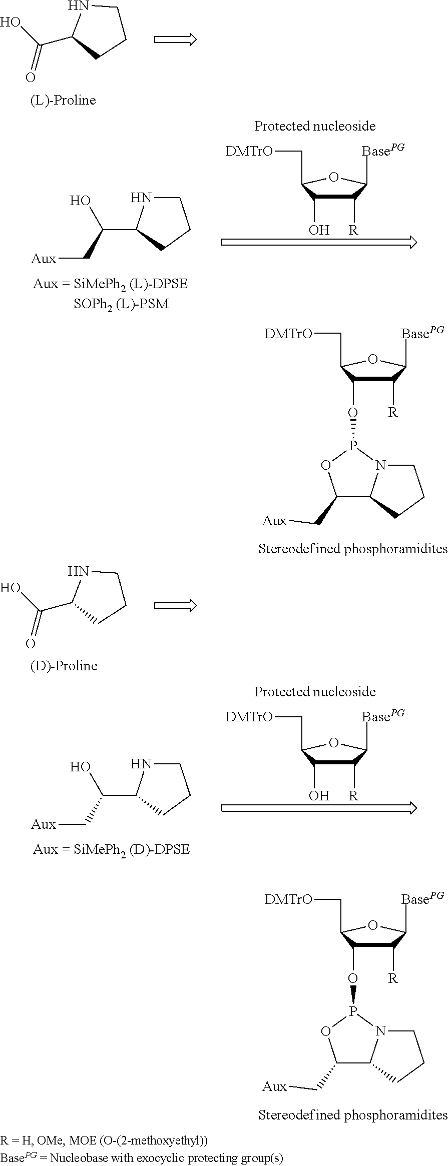

In some embodiments, the chemical purity of the stereodefined phosphoramidite is determined including by ³¹P NMR. Absolute stereogenic configuration can be determined by a combination of ³¹P NMR, ¹H NMR, and ¹³C NMR. As the stereochemical configuration of the protected nucleoside and chiral auxiliaries are fixed, there are 2 possible diastereomers (trans and cis) that can result from the phosphitylation reaction, of which the trans form is typically predominant, with the cis form being present as a minor impurity.

Certain Possible Stereoisomers Resulting from Starting Material Coupling Reactions

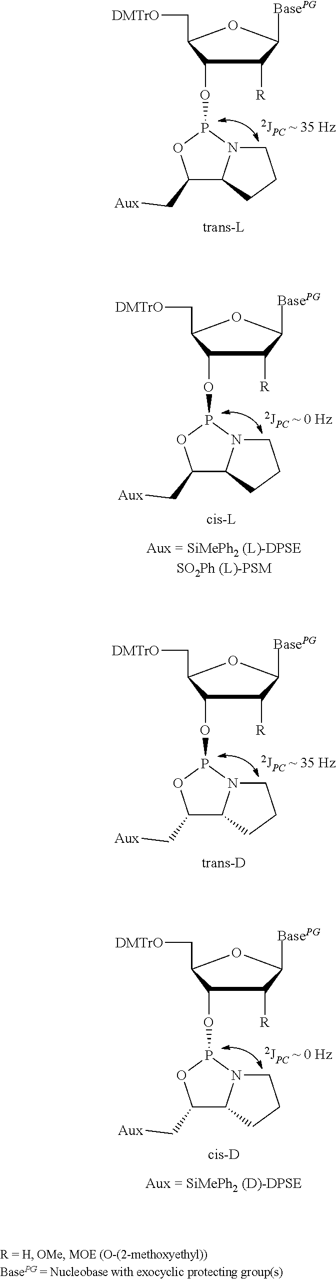

Throughout the manufacture of WVE-003, e.g., drug substance, various in-process controls are applied, e.g., as described below. Upon completion of the synthesis and the cleavage and deprotection stages, the presence of WVE-003 in the resulting crude oligonucleotide is identified by LC/MS (liquid chromatography mass spectrometry) (e.g., 7257.9±3 Da) and its purity quantified by IP-RP-UPLC (ion pair reverse phase ultraperformance liquid chromatography) (e.g., ≥84% according to Protocol A). During purification, selected mock pools are evaluated for purity and impurities by IP-RP-UPLC. Once selected fractions are pooled and concentrated, desalting of the resulting solution is controlled via in-process measurements of conductivity (e.g., ≤50 μS/cm), pH (e.g., 6.7-7.3) and concentration.

In some embodiments, a drug substance or a drug product is manufactured meeting one or more or all of the criteria described herein. In some embodiments, a drug substance, e.g., WVE-003 or a salt form thereof, has a certain level of purity as described herein. In some embodiments, WVE-003 pentadecasodium salt has a certain level of purity as described herein. In some embodiments, WVE-003 or a salt form thereof has a certain level of purity in a composition, e.g., a pharmaceutical composition. In some embodiments, WVE-003 or a salt form thereof has a certain level of purity in a drug product. In some embodiments, a salt form is WVE-003 pentadecasodium salt. In some embodiments, WVE-003 has a level of purity of about 70%-90%, about 80%-90%, about 84%-90%, or about 70%, 71%, 72%, 73%, 74%, 75%, 76%, 77%, 78%, 79%, 80%, 81%, 82%, 83%, 84%, 85%, 86%, 87%, 88%, 89%, or 90% or more as determined by IP-RP-UPLC described herein.

Multiple lots of WVE-003 drug substance have been manufactured. In some embodiments, scale was over 20 mmol. In some embodiments, scale was multiple of about 20 mmol. In some embodiments, manufactured WVE-003 was utilized in clinical trials.

### Characterization

Among other things, the present disclosure provides technologies for characterizing and/or assessing WVE-003 or compositions thereof, e.g., a WVE-003 preparation such as a drug substance, a drug product, etc.

Products can be characterized by various technologies. For example, WVE-003 may be characterized by mass spectrometry. For one preparation, electrospray ionization mass spectrometry (ESI-MS) analysis was performed, and the theoretical average mass (free acid form) of WVE-003 is consistent with the experimentally determined value of 7257 Da.

In some embodiments, the sequence of WVE-003 was confirmed by ESI-MS/MS sequencing. For example, in one assessment, at least one sequence-relevant fragment ion was observed for 19 nucleobases in the sequence within 5 ppm error of each calculated (expected) mass. The monoisotopic mass of the full-length oligonucleotide was also experimentally verified in the ESI-MS/MS analysis (7253.3428 Da), and the identities and locations of all 20 nucleobases have been demonstrated, confirming the sequence of WVE-003.

Stereochemical identity is established through control of the starting materials for synthesis. Synthetic coupling of phosphoramidites prepared from (L)-DPSE or (L)-PSM gives rise to phosphorothioate diester linkages of Sp chirality and phosphoramidate diester linkages of Rp chirality, respectively, and those prepared from (D)-DPSE or (D)-PSM gives rise to phosphorothioate diester linkages of Rp chirality and phosphoramidate diester linkages of Sp chirality. Consistent use of stereodefined phosphoramidites for each coupling reaction allows for control of stereochemical identity of each phosphorothioate linkage. Stereochemical identity can be confirmed using various technologies in accordance with the present disclosure, for example, stereochemical identity can be confirmed by measurement using several different techniques, which in combination, and by comparison with appropriate standards, provides accurate information about, and confirmation of, this key attribute. Useful analytical techniques include NMR (¹H, ¹⁹F, ³¹P, multidimensional, etc.) and enzymatic digestion. In some instances, NMR are performed in phosphate buffer (e.g., pH 7.0). Results including ¹H, ¹⁹F and ³¹P NMR and enzymatic digestion of various preparations are consistent with product structures. In some embodiments, NMR is referenced to water based on DSS standard. Observed data from certain experiments are described below.

In some embodiments, WVE-003 drug substance was prepared at a concentration of 1 mM in 600 L of 100 atom % D₂O solution containing 100 mM NaCl, 0.05 mM EDTA and 10 mM phosphate buffer (pH 7.0). In some embodiments, spectra were recorded at 334 K. In some embodiments, sodium trimethylsilylpropanesulfonate (DSS) in phosphate buffer was used as an external reference standard. In some embodiments, ¹H NMR spectra were calibrated indirectly based on DSS peak at 0 ppm. The ¹H NMR spectrum contains signals consistent with the WVE-003 structure and the chemical shifts of the individual regions of signals are consistent with the proposed structure. In some embodiments, ³¹P NMR spectrum of WVE-003, e.g., a prepared drug substance lot, was acquired and recorded at 161.98 MHz for phosphorus. In some embodiments, ³¹P NMR spectra were calibrated indirectly using the united scale based on ¹H NMR spectra of external standard where DSS peak was set as 0 ppm. In some embodiments, a sharp signal around 2 ppm is coming from the phosphate buffer used. The total of 19 PS/PN/PO peaks are consistent with the stereodefined mixed PO/PS/PN backbone of WVE-003 drug substance.

| Chemical Shift (ppm) | Integral | Possible Assignment |
|---|---|---|
| 55.86 | 1 | PS (phosphorothioate) |
| 55.41 | 1 | PS |
| 55.38 | 1 | PS |
| 55.27 | 1 | PS |
| 55.23 | 1 | PS |
| 55.12 | 1 | PS |
| 55.04 | 1 | PS |
| 54.89 | 1 | PS |
| 54.88 | 1 | PS |
| 54.85 | 1 | PS |
| 54.83 | 1 | PS |
| 54.59 | 1 | PS |
| 54.49 | 1 | PS |
| −1.03 | 1 | PO (phosphodiester) |
| −1.13 | 1 | PO |
| −1.89 | 1 | PN (N-(1,3-dimethylimidazolidin-2- |
| | | ylidenyl) phosphoramidate) |
| −2.30 | 1 | PN |
| −2.52 | 1 | PN |
| −2.69 | 1 | PN |

In some embodiments, stereochemical identity of WVE-003 was confirmed by enzymatic digestion assay.

In some embodiments, stereochemical purity is presented as the percentage of correct diastereomer associated with the assigned stereochemical identity to the total diastereomeric mixture. This is inclusive of small quantities of other diastereomers that may be present. The diastereoselectivity of each (L)- or (D)-DPSE phosphoramidite to its stereodefined phosphorothioate or phosphoramidate linkage in WVE-003 is very high and the overall stereochemical purity of the oligonucleotide is a product of the combination of the diastereoselectivities of these 13 phosphorothioate and 4 phosphoramidate linkages.

In some embodiments, diastereoselectivities of chiral linkages are assessed through preparation of corresponding dimers. The synthesis conditions used to produce each sequence dimer are identical to those applied to the whole molecule. These dimer units are separated and analyzed by RP-UPLC conditions and Rp and Sp diastereomers are separated and quantified. In some embodiments, WVE-003 sequence dimers and their respective references were analyzed by Ultra Performance Liquid Chromatography (UPLC) for purity and/or by Ultra Performance Liquid Chromatography-Mass Spectrometry (UPLC-MS) for mass confirmation of Sp and Rp diastereomers. The analysis of these samples was completed on the crude material to show the representative diastereoselectivity of the linkage during the synthesis process. The stereochemical purity of various dimers analyzed were presented below. All the sequence specific dimers were synthesized with the same process conditions as WVE-003, accurately representing the phosphorothiolated and phosphoramidated internucleotide linkages.

### Diastereoselectivity Summary of WVE-003 Drug Substance Model Dimers

| Dimer | % Sp | % Rp |
|---|---|---|
| A*SG | 98.21 | 1.79 |
| C*SA | 97.35 | 2.65 |
| C*ST | 98.29 | 1.71 |
| G*Rm5Ceo | 1.52 | 98.48 |
| G*SC | 98.41 | 1.59 |
| G*ST | 98.47 | 1.53 |
| Geon001Rm5Ceo | 0.13 | 99.87 |
| m5Ceo*STeo | 99.84 | 0.16 |
| m5Ceon001RAeo | 0.16 | 99.84 |
| mA*ST | 99.77 | 0.23 |
| mG*SmU | 99.77 | 0.23 |
| mGn001RmA | 0.31 | 99.69 |
| mUn001RmU | 0.15 | 99.85 |
| T*RA | 1.48 | 98.52 |
| T*SC | 98.31 | 1.69 |
| T*SG | 98.28 | 1.72 |

WVE-003 stereochemical purity can be attributed to the product of the stereochemical purities of each of the 13 phosphorothioate and the 4 phosphoramidate linkages in the sequence:

| Dimer | % Purity |
|---|---|
| A*SG | 98.21 |
| A*SG | 98.21 |
| C*SA | 97.35 |
| C*ST | 98.29 |
| G*Rm5Ceo | 98.48 |
| G*SC | 98.41 |
| G*ST | 98.47 |
| Geon001Rm5Ceo | 99.87 |
| m5Ceo*STeo | 99.84 |
| m5Ceon001RAeo | 99.84 |
| mA*ST | 99.77 |
| mG*SmU | 99.77 |
| mGn001RmA | 99.69 |
| mUn001RmU | 99.85 |
| T*RA | 98.52 |
| T*SC | 98.31 |
| T*SG | 98.28 |
| Stereochemical Purity | 82.69% |

In some embodiments, as demonstrated herein, the diastereomeric purity of WVE-003 is at about 82.7% by dimer modeling. In some embodiments, UPLC analysis is utilized in dimer modeling. As demonstrated herein, all 17 linkages are ≥97% stereopure, 9 linkages are ≥97% stereopure, and 7 linkages are ≥99% stereopure. In some embodiments, as demonstrated herein, an average stereochemical purity of all linkages is 98.9%.

In some embodiments, a reference standard, e.g., one characterized by NMR, enzymatic digestion, etc. is used to determine stereochemical identity by UPLC as part of batch release. In some embodiments, the provided technology provides reference standards. In some embodiments, a reference standards has purity (e.g., as assessed by Protocol A) and/or stereopurity (e.g., as assessed by Protocol B and/or dimer modeling as described herein).

FTIR spectrum of WVE-003 was obtained with an attenuated total reflectance (ATR) sampling accessory, with major absorbance bands at 1636 cm⁻¹ and 1600 cm⁻¹, absorption peak at 2938 cm⁻¹, and broad peaks at 3199 cm⁻¹ and 3339 cm⁻¹. The FTIR results is consistent with the structure of WVE-003.

Counter ions can be analyzed in accordance with the present disclosure. In some embodiments, sodium content is assessed by ICP-OES. In some embodiments, a sodium content value of 4.5% has been determined by ICP-OES for a WVE-003 drug substance, which is in agreement with the theoretical sodium content value of 4.5% (w/w).

For various preparations, WVE-003 drug substance is a white to off-white powder. The pH of WVE-003 drug substance in purified water was found to be 6.0-8.0. In some embodiments, it is about 6.5-7.5. In some embodiments, it is about 6.4. In some embodiments, it is about 6.5. In some embodiments, it is about 6.6. In some embodiments, it is about 6.7. In some embodiments, it is about 6.8. In some embodiments, it is about 6.9. In some embodiments, it is about 7.0. In some embodiments, it is about 7.1. In some embodiments, it is about 7.2. In some embodiments, it is about 7.3. In some embodiments, it is about 7.4.

The molar extinction coefficient of WVE-003 drug substance was experimentally determined in water to be 181181 M⁻¹cm⁻¹ at 260 nm, and was utilized to convert UV absorptions to concentrations, from which amounts may be calculated. It equates to an absorptivity factor of about 25.0 OD/mg.

In various embodiments, impurities are controlled at low levels as described herein. For example, in some embodiments, impurity level is lower than about 30%, about 25%, or about 20% as described herein. In some embodiments, impurities include various oligonucleotide impurities. Various technologies may be utilized to identify, characterize and/or assess impurities, e.g., mass spectrometry, LC, UV, etc.

WVE-003 drug substance was assessed in GLP in vitro genotoxicity studies and in an in vivo micronucleus study. No toxicity was observed.

Various preparations of WVE-003 drug substance have met specified limits for residual solvents, including acetonitrile, toluene and pyridine (ICH Class 2), and/or elemental impurities, including various elements in ICH Class 1, 2A, 3, etc. Elemental impurities for various preparations were within limits.

Release specifications of WVE-003 drug substance and/or drug product may include one or more specifications described herein, e.g., appearance (e.g., visual, white to off white powder), sequence identity (e.g., by MS/MS), molecular weight, stereochemical identity (e.g., IP-RP-UPLC), purity (e.g., area % by IP-RP-UPLC (e.g., Protocol A)), impurities (e.g., area % by IP-RP-UPLC (e.g., Protocol A)), sodium content (e.g., by ICP-OES, etc.), water content (% w/w; e.g., USP <921> and/or Ph. Eur. 2.5.12, etc.), assay (e.g., free acid, anhydrous, by UV), pH (e.g., of solution in purified water; e.g., USP <791> and/or Ph. Eur. 2.2.3, etc.), residual solvents (e.g., by gas chromatography), elemental impurities (e.g., by ICP-MS), bacterial endotoxins (e.g., by USP <85> and/or Ph. Eur 2.6.14), bioburden (total microbial aerobic count, total yeast and bolds count, etc. by USP <61>) and/or Ph. Eur 2.6.12), etc.

In some embodiments, the identity of WVE-003, e.g., a drug substance, is determined by liquid chromatography mass spectrometry (LC-MS). Samples and analytical reference material are prepared in water and injected on a Waters Acquity BEH C18 column. Analysis involves a gradient of mobile phase A (hexafluoroisopropanol [HFIP] and triethylamine [TEA] in water) and mobile phase B (acetonitrile). A summary of method parameters is provided below:

| Parameter | | Value | | |
|---|---|---|---|---|
| Column | | Waters Acquity BEH C18 Column, 1.7 μm, | | |
| | | 2.1 mm × 50 mm, or equivalent | | |
| Column Temperature | | 55° C. | | |
| Mobile Phase A | | 100 mM HFIP, 10 mM TEA in water | | |
| Mobile Phase B | | Acetonitrile | | |
| | | Time | | |
| | | (Min) | % MPA | % MPB |
| | Gradient | Initial | 99.0 | 1.0 |
| | | 2.00 | 99.0 | 1.0 |
| | | 11.00 | 85.0 | 15.0 |
| | | 13.00 | 50.0 | 50.0 |
| | | 14.00 | 50.0 | 50.0 |
| | | 14.10 | 99.0 | 1.0 |
| | | 15.00 | 99.0 | 1.0 |

| | | |
|---|---|---|
| Run Time | 15 | min |
| Injection Volume | 2 | μL |
| Flow Rate | 0.8 | mL/min |
| Detection | 260 | nm |
| Wavelength | | |

| | Parameter | Value |
|---|---|---|
| MS Acquisition | Scan Type | Full MS Scan |
| Parameters | Scan Range (m/z) | 500-3000 |
| | Polarity | Negative |

In some embodiments, system suitability is confirmed when the molecular weight (deconvoluted mass) for full length product (FLP) for 3 initial injections of the analytical reference material should be 7258±3 Da for 4WV-003. In addition, blank chromatogram has no interfering peaks other than the solvent front and gradient shift.

In some embodiments, identity by sequencing of WVE-003, e.g., in a drug substance, is determined by high resolution mass spectrometry (MS) and tandem mass spectrometry (MS/MS). A useful procedure is described below as an example. Samples are prepared in water and directly infused into the mass spectrometer for MS/MS analysis. Analysis involves an isocratic LC-method with a mixture of mobile phase A (hexafluoroisopropanol [HFIP] and triethylamine [TEA] in LC-MS grade water) and mobile phase

| Parameter | Value |
|---|---|
| Column | Flow Injection |
| Mobile Phase A | 400 mM HFIP, 15 mM TEA in water |
| Mobile Phase B | Acetonitrile |

| | Time (Min) | % MPA | % MPB |
|---|---|---|---|
| Isocratic Mobile | 1 minute | 70.0 | 30 |
| Phase Composition | | | |

| Injection Rate | 5 μL/min (using syringe) | |
|---|---|---|
| Flow Rate | 0.3 | mL/min |
| Detection | 260 | nm |
| Wavelength | | |

| | | |
|---|---|---|
| | Parameter | Value |
| MS Acquisition | Scan Type | Full MS Scan |
| Parameters | Scan Range (m/z) | 500-3000 |
| | Polarity | Negative |
| MS/MS Acquisition | MS/MS Scan Type | Targeted Fragmentation Scan |
| Parameters | Polarity | Negative |

| | | |
|---|---|---|
| Fragment ion | Charge State m/z 725 + | Scan Range |
| selected | 1.5 Da (z = −10) | 200-3000 |
| Fragment ion | Charge State m/z 1036 + | Scan Range |
| selected | 1.5 Da (z = −7) | 200-3000 |
| Fragment ion | Charge State m/z 1209 = | Scan Range |
| selected | 1.5 Da (z = −6) | 800-1600 |

In some embodiments, system suitability is confirmed when the m/z charge states used in the sequencing experiment are within ±1 Da of the theoretical values, the exact full length product mass is 7253.3302±0.1000 amu, and the mass spectrum in MS mode matches a reference spectrum in the method.

In some embodiments, the sodium content of WVE-003 preparation, e.g., drug substance, is determined by inductively coupled plasma optical emission spectroscopy (ICP-OES). Sample material in solution is introduced by pneumatic nebulization into radio frequency plasma where energy transfer processes cause desolvation, atomization, and ionization. Sodium emits light during this process and the instrument measures absorbance to quantitate levels of sodium. In some embodiments, a sodium content (anhydrous basis) is about 3.9-5.2% (w/w). In some embodiments, it is about 4-5%. In some embodiments, it is about 4.3%-4.7%. In some embodiments, it is about 4.3%. In some embodiments, it is about 4.4%. In some embodiments, it is about 4.5%. In some embodiments, it is about 4.6%. In some embodiments, it is about 4.7%.

In some embodiments, the present disclosure provides technologies for assessing purity and/or impurities of a WVE-003 preparation or composition. In some embodiments, purity and impurities of WVE-003 drug substance are determined by ion-pair reversed-phase UPLC (IP-RP-UPLC) using a Waters BEH C18 Column. A useful procedure is described below as example (Protocol A). The separation is provided using a gradient of mobile phase A (hexafluoroisopropanol [HFIP] and triethylamine [TEA] in water) and mobile phase B (50% acetonitrile in water). A summary of method parameters is provided below as an example (Set A parameters). In some embodiments, system suitability is established by the absence of interfering peaks in the blank as well as acceptance criteria for 1RSD for retention time, peak area % and peak area for the initial injections and all injections of WVE-003 analytical reference material (system suitability standard). In some embodiments, all individual impurities 0.10, as a function of relative retention time to the main peak, as well as total of all impurities 0.10 area are reported. In some embodiments, WVE-003 identity is confirmed by comparing relative retention time with system suitability standard. The method has been determined to be stability indicating in method development studies.

| Parameter | Value | |
|---|---|---|
| Column | Waters BEH Oligonucleotide C18 Column, 1.7 μm, | |
| | 2.1 mm × 100 mm, or equivalent | |
| Mobile Phase A | 100 mM HFIP, 10 mM TEA in water | |
| Mobile Phase B | 50% Acetonitrile in water | |
| Injection Volume | 2 | μL |
| Flow Rate | 0.5 | mL/min |
| Run Time | 30 | minutes |
| Detection | 260 | nm |
| Wavelength | | |

| | Time | % A | % B | Curve |
|---|---|---|---|---|
| Gradient | Initial | 94 | 6 | N/A |
| | 1 | 94 | 6 | 6 |
| | 8 | 84 | 16 | 6 |
| | 20 | 74 | 26 | 6 |
| | 25 | 0 | 100 | 6 |
| | 26 | 0 | 100 | 6 |
| | 26.5 | 94 | 6 | 1 |
| | 30 | 94 | 6 | 1 |

In some embodiments, purity of a WVE-003 preparation, e.g., a drug substance, a drug product, etc., is about 84% or more (e.g., assessed by Protocol A (% area)). In some embodiments, it is about 85% or more. In some embodiments, it is about 86% or more. In some embodiments, it is about 87% or more. In some embodiments, it is about 88% or more. In some embodiments, it is about 80%-90%. In some embodiments, it is about 84%-90%. In some embodiments, it is about 84%-88%. In some embodiments, total impurities arc about or no more than about 16% (e.g., assessed by Protocol A (% area)). In some embodiments, total impurities are about or no more than about 15%. In some embodiments, total impurities are about or no more than about 14%. In some embodiments, total impurities are about or no more than about 13%. In some embodiments, total impurities are about or no more than about 12%. In some embodiments, sum of WVE-003 and total impurities is about 99%-101%.

In some embodiments, assay of a WVE-003 preparation, e.g., a drug substance, a drug product, etc., is determined by UV spectrophotometry. A useful procedure is described herein as an example. A quantity of WVE-003 drug substance is accurately weighed and dissolved in water in volumetric glassware. The solution absorbance is determined at 260 nm, and this value is converted to a concentration of WVE-003 drug substance using the molar extinction coefficient (MEC; 181,181 M⁻¹cm⁻¹ and 25.0 OD/mg). The quantity of the weighed material is corrected by subtraction of the water content of the sample (e.g., by Karl Fischer titration USP <921> Method 1c) and subtraction of the sodium content. The Assay (%) is then determined as a ratio of the WVE-003 quantity measured by absorbance to the theoretical prepared quantity based on the accurate weight of the sample corrected for water and sodium content. In some embodiments, assay of a WVE-003 preparation, e.g., a drug substance, a drug product, etc., is about 100%±10%. In some embodiments, it is about 95%-105%. In some embodiments, it is about 95%. In some embodiments, it is about 96%. In some embodiments, it is about 97%. In some embodiments, it is about 98%.

In some embodiments, water content of a WVE-003 preparation, e.g., a drug substance, a drug product, etc., is determined by coulometric titration (Karl Fischer). In some embodiments, system suitability is established by required adequate recovery from a sodium tartrate monohydrate certified control standard of known water content. In some embodiments, water content of a WVE-003 preparation, e.g., a drug substance, a drug product, etc., is no more than about 15%, 10%, 5% or 1% (w/w). In some embodiments, it is no more than about 15%. In some embodiments, it is no more than about 10%. In some embodiments, it is no more than about 9%. In some embodiments, it is no more than about 8%. In some embodiments, it is no more than about 7%. In some embodiments, it is no more than about 6%. In some embodiments, it is no more than about 5%. In some embodiments, it is no more than about 1%.

In some embodiments, residual solvents within a WVE-003 preparation, e.g., a drug substance, a drug product, etc. are quantified using gas chromatography with flame ionization detection (GC-FID). In some embodiments, samples are prepared by being mixed well, weighed into crimped vials and dissolved in solvent. Sample is then introduced via direct injection and quantitation is performed as a limit test by comparison to an external standard. Blanks and calibration verifications are analyzed at appropriate intervals.

In some embodiments, the present disclosure provides technologies for confirming stereochemical identity of WVE-003 or a diastereomer thereof (e.g., with respect to one or more chiral linkage phosphorus centers). In some embodiments, stereochemical identity of WVE-003, e.g., in a WVE-003 preparation, a drug substance, a drug product, etc. is determined by IP-RP-UPLC using a Waters BEH C18 Column. A useful procedure is described below as an example (Protocol B). In some embodiments, the separation of WVE-003 from closely related diastereomeric impurities is provided using a gradient of mobile phase A (triethylamine acetate [TEAA] in water) and mobile phase B (50% acetonitrile in water). A summary of method parameters is provided below (Set B parameters). In some embodiments, system suitability is established by the absence of interfering peaks in the blank as well as acceptance criteria for % RSD for retention time and peak area for the initial injections of WVE-003 analytical reference material, for 00 difference between the trailing standard peak area and the average of the initial injections of standard, and % RSD for retention time for all standard injections. Method performance is also verified by demonstrating resolution of a reference sample from a closely related diastereomer. For stereochemical identification, stereochemical identity is confirmed if the % retention time difference of the main peak is no more than 3% from retention time of a structurally characterized reference material.

| Parameter | Value |
|---|---|
| Column | Waters BEH Oligonucleotide C18 Column, |
| | 1.7 μm, 2.1 × 150 mm, or |
| | equivalent |
| Mobile Phase A | 40 mM TEAA in water |
| Mobile Phase B | 50% Acetonitrile in water |
| Injection Volume | 2 μL |
| Flow Rate | 0.1 mL/min |
| Run Time | 60 minutes |
| Column Temperature | 18° C. |
| Detection | 260 nm |
| Wavelength | |

| | Time | % A | % B | Curve |
|---|---|---|---|---|
| Gradient | Initial | 68 | 32 | N/A |
| | 2 | 68 | 32 | 6 |
| | 10 (0.05 mL/min) | 66.5 | 33.5 | 6 |
| | 35 (0.05 mL/min) | 66 | 34 | 6 |
| | 45 (0.05 mL/min) | 65 | 35 | 6 |
| | 49 | 40 | 60 | 6 |
| | 51 | 40 | 60 | 6 |
| | 52 | 68 | 32 | 1 |
| | 60 | 68 | 32 | 1 |

In some embodiments, bacterial endotoxins are determined according to USP <85> and/or Ph. Eur. 2.6.14. In some embodiments, bioburden, e.g., both total aerobic microbial count (TAMC) and total yeast and mold counts (TYMC), is determined according to USP <61> and/or Ph. Eur. 2.6.12.

WVE-003 drug substance is packaged in a suitable container, e.g., sterile high-density polyethylene (HDPE) bottles with polypropylene screw closures, labeled and sealed in a protector, e.g., Mylar foil pouches which can provide a gas/moisture barrier with high levels of abrasion and puncture resistance.

Various batches of WVE-003 preparations have been manufactured. Certain preparations was utilized in nonclinical studies including GLP toxicology studies. Certain preparations were manufactured for clinical studies. Manufactured preparations are stable. In some embodiments, WVE-003 or a composition thereof is stored at about −20° C. In some embodiments, a long-term storage condition is at about −20° C., e.g. ±5° C. In some embodiments, a storage is at about 5° C., e.g., ±3° C.

### Drug Product

In some embodiments, the present disclosure provides a WVE-003 drug product. In some embodiments, a WVE-003 drug product comprises a WVE-003 drug substance, e.g., WVE-003 pentadecasodium salt manufactured using a process described above. In some embodiments, a WVE-003 drug product consists of WVE-003 drug substance, e.g., WVE-003 pentadecasodium salt, as a lyophilized solid in a vial. In some embodiments, a vial is a 10 mL vial. In some embodiments, a vial is a 10 mL USP/Ph. Eur. Type 1 clear glass vial sealed with a FluroTec® coated elastomeric rubber stopper and an aluminum overseal with a blue matte flip-off button. In some embodiments, a vial contains WVE-003 for a single dose as described herein. In some embodiments, a vial contains WVE-003 equivalent to about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg WVE-003 free acid form. In some embodiments, a vial contains about 20 mg WVE-003 (unless specified otherwise, based on free acid form). In some embodiments, vials filled with 2.53 mL of WVE-003 drug substance at 8 mg/mL concentration in water for injection (WFI) are lyophilized and backfilled with nitrogen gas, NF upon completion of the lyophilization cycle. In some embodiments, a WVE-003 drug product vial contains about 20 mg of WVE-003 drug substance. In some embodiments, a WVE-003 drug product vial contains about 30 mg of WVE-003 drug substance. In some embodiments, a WVE-003 drug product vial contains about 40 mg of WVE-003 drug substance. In some embodiments, a WVE-003 drug product vial contains about 50 mg of WVE-003 drug substance. In some embodiments, a WVE-003 drug product vial contains about 60 mg of WVE-003 drug substance. In some embodiments, a WVE-003 drug product vial contains about 70 mg of WVE-003 drug substance. In some embodiments, a WVE-003 drug product vial contains about 80 mg of WVE-003 drug substance. In some embodiments, a WVE-003 drug product vial contains about 90 mg of WVE-003 drug substance. In some embodiments, a WVE-003 drug product vial contains about 100 mg of WVE-003 drug substance. In some embodiments, a WVE-003 drug product vial contains about 110 mg of WVE-003 drug substance. In some embodiments, a WVE-003 drug product vial contains about 120 mg of WVE-003 drug substance. In some embodiments, a WVE-003 drug product vial contains about 130 mg of WVE-003 drug substance. In some embodiments, a WVE-003 drug product vial contains about 140 mg of WVE-003 drug substance. In some embodiments, a WVE-003 drug product vial contains about 150 mg of WVE-003 drug substance. In some embodiments, a WVE-003 drug product vial contains about 160 mg of WVE-003 drug substance. In some embodiments, a WVE-003 drug product vial contains about 170 mg of WVE-003 drug substance. In some embodiments, the present disclosure provides a pharmaceutical composition comprising or delivering WVE-003 or a pharmaceutically acceptable salt form thereof and a pharmaceutically acceptable carrier. In some embodiments, the present disclosure provides a pharmaceutical composition comprising WVE-003 pentadecasodium salt and a pharmaceutically acceptable carrier. In some embodiments, a pharmaceutical composition consists of a WVE-003 drug substance and a pharmaceutically acceptable carrier. In some embodiments, a pharmaceutically acceptable carrier is aCSF. In some embodiments, each WVE-003 drug product vial contains WVE-003 drug substance for a single dose. In some embodiments, WVE-003 drug substance is reconstituted and diluted in artificial cerebral spinal fluid (aCSF) as a sterile, preservative-free solution at the clinical site prior to intrathecal administration.

In some embodiments, a WVE-003 drug product contains no preservatives, inactive ingredients, or excipients.

In some embodiments, WVE-003 drug product is a white to off-white solid with no visible material and essentially free of particulates upon reconstitution. In some embodiments, pH of a 20 mg WVE-003 in 2 mL artificial cerebrospinal fluid (aCSF) solution is about 6.0-8.0. In some embodiments, pH of a 20 mg WVE-003 in 2 mL aCSF solution is about 6.4-7.2.

In some embodiments, a freezing temperature at about −45° C., primary and secondary drying temperatures at about −14° C. and 25° C., and 100 mTorr pressure are utilized. Other skilled in the art appreciate that other temperatures and/or pressure may also be utilized.

In a preparation, approximately 75 grams of WVE-003 drug substance is formulated with water for injection, Ph. Eur./USP to achieve 8 mg/mL solution. Approximately, 2.53 mL each of this solution is lyophilized to a powder yielding a nominal value of 20 mg drug/vial. A drug product lot may be made by pooling multiple drug substance lots. In some embodiments, as an example the total number of vials calculated from the amount of drug substance is as follows:

The purity, sodium content, and water content of drug substance was 86%, 4.3%, and 8%, respectively.

The drug substance (DS) amount used for manufacturing drug product (DP) batch is 75.04 g.

$$The total amount of oligonucleotide in DS for the batch = Amount \\ \\ of DS ( g ) \times 86 \% Purity / 100 \% \times ( 100 \% - 4.3 \% Sodium Content ) / 100 \% \times ( 100 \% - 8 \% Water Content ) / 100 \% = 75.04 g \times 0.86 \times ( 0.957 ) \times ( 0.92 ) \times 1000 mg / g = 56 , 788.38 mg .$$

$$The total batch size of drug product at 20.24 mg / vial = Amount of DS batch / DP vial content = 56 , 788 mg / 20.24 mg = 2 , 805 vials .$$

In some embodiments, as an example the total number of vials and batch volume calculated from the amount of drug substance is as follows:

The purity, sodium content, and water content of drug substance was 86%, 4.3%, and 8%, respectively.

The drug substance (DS) amount used for manufacturing drug product (DP) batch is 75.04 g.

$$The total amount of oligonucleotide in DS for the batch = Amount of DS ( g ) \times 86 \% Purity / 100 \% \times ( 100 \% - 4.3 \% Sodium Content - 8 \% Water Content ) / 100 \% = 75.04 g \times 0.86 \times 0.877 \times 1000 mg / g = 56 , 596.67 mg .$$

$$The total batch size of drug product at 20.2 4 mg / vial = Amount of DS batch / DP vial content = 56 , 596 mg / 20.24 mg = 2 , 824 vials .$$

In some embodiments, the following formula is utilized for calculating batch volume:

$$Theoretical Batch Volume ( mL ) = Theoretical total DS in mg \times ( Purity by UPLC ( \% ) ) / 100 ) \times [ ( 100 \% - Sodium Content ( \% ) - Water Content ( \% ) ) / 100 \% ] \div Concentration$$

For example, in one example, Theoretical Batch Volume (mL)=75000 mg×0.86×0.877÷8.0 mg/mL=7,070 mL.

Those skilled in the art appreciate that in various circumstances these example methods can provide vials with about the same amount of WVE-003 and can provide administration and delivery of about the same amount of WVE-003 to patients.

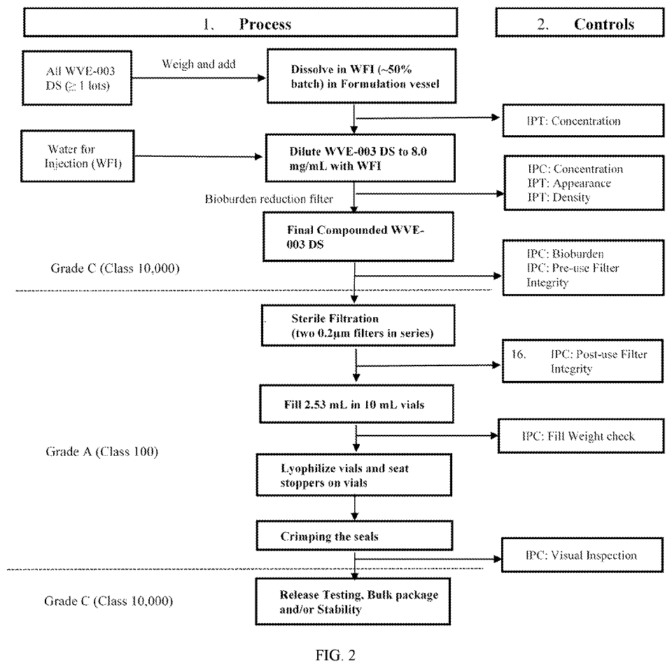

A follow diagram for a WVE-003 drug product manufacturing process is provided in FIG. 2 as an example. WVE-003 drug substance (DS) containers are thawed at 2-8° C. followed by equilibrating at room temperature (RT). Employing the purity factor, sodium content and % moisture, pure full-length product (FLP) is calculated as:

$$Pure FLP = [ DS ( g ) \times \frac{purity}{100} \times \frac{( 100 \% - sodium content \% )}{100 \%} \times \frac{( 100 \% - moisture \% )}{100 \%} ]$$

In some embodiments, a pure full-length product (FLP) is calculated as:

$$Pure FLP = [ DS ( g ) \times \frac{purity}{100} \times \frac{( 100 \% - sodium content \% - moisture \% )}{100 \%} ]$$

Those skilled in the art appreciate that in various circumstances (e.g., levels as described herein for purity, sodium content %, moisture (water content) %, etc., and methods as described herein for assessing purity (e.g., UPLC as described herein), sodium content % (e.g., ICP-OES as described herein), moisture % (e.g., Karl Fischer (e.g., USP <921>) as described herein), etc.), the two calculations can provide about the same results. In cases where multiple lots of drug substance are to be pooled, then a purity correction factor that is based on the quantity of DS from each lot is determined. Depending on pure FLP weight, the batch volume to manufacture 8 mg/mL solution is calculated.

WFI in the amount of ˜50% of the calculated batch volume is added to the formulation vessel by weight. The DS containers are weighed and DS from each container is transferred into the formulation vessel. Appropriate WFI rinses are used to ensure complete removal of DS from containers. The empty DS containers are dried and weighed to calculate total DS added to the formulation vessel.

The DS is mixed adequately and an in-process sample to measure concentration by ultraviolet (UV) assay is taken. The remaining solution mass in the formulation is weighed. Based on the results of purity corrected WVE-003 drug substance concentration, and solution mass, the required amount of WFI to accomplish WVE-003 drug substance final concentration of 8.0 mg/mL is calculated as follows:

$$[ DS conc . ( \frac{mg}{mL} ) \times \frac{Solution mass ( g )}{final DS conc . ( 8 \frac{mg}{mL} )} ] - Solution mass ( g )$$

Required amount of WFI is then slowly added directly to the formulation vessel and mixed to ensure homogenous solution is formed. In-process samples are collected for appearance, concentration by UV spectrophotometry, and density, which should meet various specifications. This final compounded formulation undergoes bioburden reduction filtration following which it can be stored overnight at 2-8° C., if required.

In some embodiments, a pre-filtration bioburden sample is taken prior to sterile filtration of the compounded bulk solution. The final compounded formulation is sterile filtered through two 0.2 pm filters in series prior to filling. Sterile filter units are checked for filter integrity by bubble point method prior to and post filtration.

The target fill weight is determined based on target fill volume of 2.53 mL and measured density. Filling occurs with periodic fill checks and filled vials with stoppers in lyo-position are placed on lyophilization-trays. HEPA carts are used for aseptic transfer of lyophilization-trays into the sterilized lyophilizer. In some embodiments, lyophilization cycle parameters are those provided below.

| Lyo | | TIME | TEMP. | | Pressure Set | Alarm Value |
|---|---|---|---|---|---|---|
| EP | Cycle Phase | (Min) | (° C.) | Activity | point (mTorr) | (mTorr) |
| 1 | Load | N/A | 5.0 | Load vials | N/A | N/A |

Freezing

| | | | | | | |
|---|---|---|---|---|---|---|
| 2 | Hold | * | 5.0 | Hold | NA | NA |
| 3 | Ramp | 135 | −45.0 | Ramp Rate: 0.37° C./min | N/A | N/A |
| 4 | Hold | 240 | −45.0 | Hold | N/A | N/A |

Primary Drying

| | | | | | | |
|---|---|---|---|---|---|---|
| 5 | Ramp | 90 | −14.0 | Ramp Rate: 0.342° C./min | 100 | 150 |
| 6 | Hold | 1200 | −14.0 | Hold | 100 | 150 |

Secondary Drying

| | | | | | | |
|---|---|---|---|---|---|---|
| 7 | Ramp | 80 | 25 | Ramp Rate: 0.49° C./min | 100 | 150 |
| 8 | Hold | 480 | 25 | Hold | 100 | 150 |

Upon completion of lyophilization, chamber pressure is increased to 9.6 psi to backfill chamber with sterile nitrogen (N2). The stoppers are seated prior to de-shelving the trays and seals are applied via crimping. The vials are then visually inspected before sampling for release, stability testing, and packaging or storage.

Process controls and in-process control tests and acceptance limits are applied during the manufacture of WVE-003 drug product. A primary control of critical steps during the manufacture of WVE-003 drug product is exerted via in-process concentration measurements via ultraviolet (UV) spectrophotometry. The in-process concentration measurement is used to calculate the amount of WFI required to dilute compounding solution to a final concentration of 8 mg/mL WVE-003 drug substance. Appropriate dilution is confirmed by measuring the concentration of final bulk drug solution. Microbial control is exerted by measuring pre-filtration bioburden and analyzing pre- and post-filtration integrity of the sterilizing filters. Drug product filled vials are subjected to periodic weight checks during the filling process and 100% visual inspection prior to sampling for release, bulk packaging, and/or stability.

Release specifications of WVE-003 drug product may include one or more specifications described herein, e.g., appearance (e.g., visual; white to off white solid), appearance after reconstitution (e.g., visual), identity (e.g., by LC-MS, retention time of IP-RP-UPLC, mass, etc.), purity (e.g., area % by IP-RP-UPLC (e.g., Protocol A)), impurities (e.g., area % by IP-RP-UPLC (e.g., Protocol A)), assay (e.g., % label claim; e.g., free acid, anhydrous, by UV), pH (e.g., after reconstitution: reconstitute 20 mg vial with 2 mL of aCSF diluent; e.g., USP <791> and/or Ph. Eur. 2.2.3), osmolality (e.g., after reconstitution: reconstitute 20 mg vial with 2 mL of aCSF diluent; e.g., USP <791> and/or Ph. Eur. 2.2.3), bacterial endotoxin (e.g., USP <85> and/or Ph. Eur. 2.6.14), sterility (e.g., USP <71> and/or Ph. Eur. 2.6.1), uniformity of dose (e.g., USP <905> and/or Ph. Eur. 2.9.40), particulate matter (e.g., USP <788> and/or Ph. Eur. 2.9.19), reconstitution time (e.g., visual), water content (e.g., % w/w; e.g., USP <921>, Ph. Eur. 2.5.12, Karl Fischer, etc.), container closure integrity testing (e.g., USP <1207>), etc. In some embodiments, a WVE-003 drug product demonstrates about the same or similar specification, e.g., purity, impurities, pH, water content, etc. as a WVE-003 drug substance as described herein.

For example, in some embodiments, purity of a WVE-003 drug product is about 84% or more (e.g., assessed by Protocol A (% area)). In some embodiments, it is about 85% or more. In some embodiments, it is about 86% or more. In some embodiments, it is about 87% or more. In some embodiments, it is about 88% or more. In some embodiments, it is about 80%-90%. In some embodiments, it is about 84%-90%. In some embodiments, it is about 84%-88%. In some embodiments, total impurities are about or no more than about 16% (e.g., assessed by Protocol A (% area)). In some embodiments, total impurities are about or no more than about 15%. In some embodiments, total impurities are about or no more than about 14%. In some embodiments, total impurities are about or no more than about 13%. In some embodiments, total impurities are about or no more than about 12%. In some embodiments, total impurities are about or no more than about 11%. In some embodiments, sum of WVE-003 and total impurities is about 99%-101%.

In some embodiments, the assay (% label claim) of the WVE-003 drug product is determined by ultraviolet (UV) spectrophotometry. Test samples are reconstituted with water, then quantitatively transferred to a volumetric flask and diluted to volume with water. The absorbance of the solution at 260 nm is measured and used, along with molar extinction coefficient (MEC), and the experimentally measured WVE-003 purity, to determine the amount of purity corrected WVE-003 drug substance per vial (mg/vial), which is then compared to the expected amount in vial, e.g., 20 mg, to obtain % label claim. In some embodiments, assay sample preparation and calculations are performed as described below:

Finished product vial is reconstituted with 2.5 mL of water to obtain a solution of 8 mg/mL. Stock Sample (0.1 mg/mL): Pipette (in duplicate) 0.32 mL of 8 mg/mL drug product into a 25 mL volumetric flask, QS with water and mix well. Working Sample (0.02 mg/mL): From each 0.1 mg/mL stock sample solution pipette 2.0 mL into a 10 mL volumetric flask. Dilute to volume with water and mix well.

Each working sample is analyzed at 260 nm (A260) with 1 reading (1 cm cuvette).

WVE-003% label claim (% LC) from A260 is calculated as follows:

$$Assay ( mg / mL ) = \frac{Abs \times Tota1 DF \times Purity}{Conversion factor \times Path Length}$$

$$Assay ( \frac{mg}{vial} ) = Assay ( \frac{mg}{mL} ) \times Reconstitution volme ( \frac{mL}{vial} )$$

$$\% LC = \frac{WVE - 003 Assay ( \frac{mg}{vial} )}{Label claim ( \frac{mg}{vial} )} \times 100$$

DF=dilution factor(s). Assay values are averaged for final reporting.

In some embodiments, % label claim is about 100% f 10%. In some embodiments, it is about 95%-105%. In some embodiments, it is about 95%. In some embodiments, it is about 96%. In some embodiments, it is about 97%. In some embodiments, it is about 98%. In some embodiments, it is about 99%. In some embodiments, it is about 100%. In some embodiments, it is about 101%. In some embodiments, it is about 102%. In some embodiments, it is about 103%. In some embodiments, it is about 104%. In some embodiments, it is about 105%. In some embodiments, it is about 106%. In some embodiments, it is about 107%. In some embodiments, it is about 108%. In some embodiments, it is about 109%. In some embodiments, it is about 110%.

In some embodiments, reconstitution time of WVE-003 drug product is determined visually upon reconstitution with 2 mL sterile, preservative-free aCSF and gentle inversion until the cake is completely dissolved. The time it takes to completely reconstitute the cake is determined using a calibrated timer.

In some embodiments, the uniformity of WVE-003 drug product dosage units is determined by UV content uniformity method. The uniformity of WVE-003 drug product dosage units is determined by reconstitution of ten vials, and if necessary, an additional twenty vials are tested. The assay (% label claim) is determined by UV spectrophotometry, as described above, for each vial. The acceptance value (AV value) is then calculated using the determined assay (% label claim) results in each tested vial as specified in the compendia.

In some embodiments, particulate matter in WVE-003 drug product is determined by reconstitution of 10 vials with 10 mL purified water, then pooling of the resulting solutions for analysis The pooled solution is then analyzed using a particle counter per the Test 1.B light obscuration test to determine the number of particles 10 μm or greater and 25 μm or greater in this solution. Calibration verification is performed prior to test sample analysis.

In some embodiments, the specification for osmolality of a WVE-003 drug product (diluted in aCSF), e.g., as measured per USP <785>/Ph. Eur. 2.2.35, aligns with the reconstitution medium (e.g., aCSF) and is consistent with the osmolality range of human cerebrospinal fluid.

In some embodiments, a WVE-003 drug product is stored at about 5° C., e.g., +3° C. In some embodiments, it is stored at about 25° C., e.g., z 2° C. In some embodiments, it can be stored at about 40° C., e.g., ±2° C. In some embodiments, it is stored at 60% RH. In some embodiments, it is stored at 75% RH. Manufactured drug products are stable for various assessed time periods and/or temperatures. In some embodiments, a WVE-003 drug product is stable for about 6, 9, 12, 15, 18, 21, 24, 27, 30, 33, 36, or more months. In some embodiments, a WVE-003 drug product is stable for about 6, 9, 12, 15, 18, 21, 24, 27, 30, 33, 36 or more months at about 5° C., e.g., +3° C. In some embodiments, a WVE-003 drug product is stable for about 6, 9, 12, 15, 18, 21, 24 or more months at about 25° C., e.g., z 2° C. (e.g., 60% RH±5%). In some embodiments, a WVE-003 drug product is stable for about 1, 2, 3, 4, 5, 6 or more months at about 40° C., e.g., +2° C. (e.g., 75% RH+5%).

In some embodiments, WVE-003 is provided in a solution composition, e.g., dissolved in aCSF. In some embodiments, diluent to be used as placebo and diluent for the reconstitution of WVE-003 (drug product vials) is an artificial cerebrospinal fluid (aCSF) solution. In some embodiments, it is in a 20 mL USP/Ph. Eur. Type I clear glass vial sealed with a serum stopper, an aluminum overseal with a white matte flip-off button. In some embodiments, a vial contains 20.8 mL of aCSF (which includes 0.8 mL overfill above the nominal fill volume of 20 mL). In some embodiments, pH of a diluent, e.g., aCSF, is about 6.8-7.8. In some embodiments, it is about 7.2-7.4. A composition of diluent is presented below:

| | | Possible | Amount per | | Concentration | |
|---|---|---|---|---|---|---|
| Component | Quality Standard | Function | vial | | mg/mL | |
| Calcium chloride, | Ph. Eur., BP, JP, | Tonicifier | 4.2 | mg | 0.21 | mg/mL |
| dihydrate | USP | | | | | |
| Magnesium chloride, | Ph. Eur., BP, USP, | Tonicifier | 3.2 | mg | 0.16 | mg/mL |
| hexahydrate Crystals | JP | | | | | |
| Potassium chloride | Ph. Eur., BP, USP, | Tonicifier | 4.4 | mg | 0.22 | mg/mL |
| | JP | | | | | |
| Sodium chloride | Ph. Eur., BP, JP, | Tonicifier | 175.4 | mg | 8.77 | mg/mL |
| | USP | | | | | |
| Sodium phosphate | Ph. Eur., BP, USP, | Buffer | 2.02 | mg | 0.101 | mg/mL |
| dibasic, anhydrous | JP | | | | | |
| Sodium phosphate | Ph. Eur., BP, USP, | Buffer | 0.9 | mg | 0.045 | mg/mL |
| monobasic, dihydrate | JP | | | | | |
| Sodium hydroxide, | Ph. Eur., NF, BP, JP | pH adjustment | As required | | As required | |
| pellets | | | | | | |

| | | | | |
|---|---|---|---|---|
| Hydrochloric acid | Ph. Eur., NF, BP, JP | pH adjustment | As required | As required |
| Water for Injection | Ph. Eur., USP | Diluent | QS | QS |

USP = United States Pharmacopeia,
NF = National Formulary,
Ph. Eur. = European Pharmacopoeia,
JP = Japanese Pharmacopoeia,
BP = British Pharmacopoeia,
QS = quantity sufficient

| | | Possible | Amount per | | Concentration | |
|---|---|---|---|---|---|---|
| Component | Quality Standard | Function | vial | | | mg/mL |
| Calcium chloride, | Ph. Eur., BP, JP, | Tonicifier | 4.368 | mg | 0.21 | mg/mL |
| dihydrate | USP | | | | | |
| Magnesium chloride, | Ph. Eur., BP, USP, | Tonicifier | 3.328 | mg | 0.16 | mg/mL |
| hexahydrate Crystals | JP | | | | | |
| Potassium chloride | Ph. Eur., BP, USP, | Tonicifier | 4.576 | mg | 0.22 | mg/mL |
| | JP | | | | | |
| Sodium chloride | Ph. Eur., BP, JP, | Tonicifier | 182.416 | mg | 8.77 | mg/mL |
| | USP | | | | | |
| Sodium phosphate | Ph. Eur., BP, USP, | Buffer | 2.1008 | mg | 0.101 | mg/mL |
| dibasic, anhydrous | JP | | | | | |
| Sodium phosphate | Ph. Eur., BP, USP, | Buffer | 0.936 | mg | 0.045 | mg/mL |
| monobasic, dihydrate | JP | | | | | |
| Sodium hydroxide, | Ph. Eur., NF, BP, JP | pH adjustment | As required | | As required | |
| pellets (1N in WFI) | | | | | | |
| Hydrochloric acid (IN | Ph. Eur., NF, BP, JP | pH adjustment | As required | | As required | |
| in WFI) | | | | | | |

| | | | | |
|---|---|---|---|---|
| Water for Injection | Ph. Eur., USP | Diluent | QS 20.8 mL | QS 1.0 mL |

USP = United States Pharmacopeia,
NF = National Formulary,
Ph. Eur. = European Pharmacopoeia,
JP = Japanese Pharmacopoeia,
BP = British Pharmacopoeia,
QS = quantity sufficient,
WFI = Water for Injection,
N = normality

### Formulations of WVE-003

In some embodiments, the present disclosure provides a formulation of a HTT oligonucleotide, e.g., WVE-003.

In some embodiments, a HTT oligonucleotide, or a salt form thereof, or a HTT oligonucleotide composition is capable of mediating an allele-specific decrease in the level, expression and/or activity of a mHTT transcript. In some embodiments, a HTT oligonucleotide, or a salt form thereof, or a HTT oligonucleotide composition is or is of WVE-003. In some embodiments, a HTT oligonucleotide, or a salt form thereof, or a HTT oligonucleotide composition is capable of mediating allele-specific knockdown of a mutant HTT transcript. In some embodiments, the present disclosure provides a chirally controlled HTT oligonucleotide composition is capable of mediating allele-specific knockdown of a mutant HTT transcript.

In some embodiments, a HTT oligonucleotide, or a salt form thereof, or a HTT oligonucleotide composition is provided as a lyophilized powder for reconstitution and dilution for administration. In some embodiments, WVE-003 preparations or formulations are in the form of a white to off-white lyophilized solid. In some embodiments solubility of WVE-003 drug substance in water determined to be at least 79.20 mg/mL as determined by UV spectrophotometry. In some embodiments, the pH of WVE-003 drug substance in purified water ranges from 6.0-8.0.

In some embodiments, a HTT oligonucleotide, or a salt form thereof, or a HTT oligonucleotide composition is provided as a liquid formulation.

In some embodiments, WVE-003 has been formulated as a lyophilized powder for reconstitution and dilution for administration. In some embodiments, WVE-003 has been formulated as a as a liquid formulation.

In some embodiments, the present disclosure provides a particular formulation of WVE-003. In some embodiments, the present disclosure provides a method of use of a particular formulation of WVE-003 at a dosage of about 30, about 60, about 90, about 120, about 150 or about 168 mg in treatment of Huntington's disease.

In some embodiments, a HTT oligonucleotide is WVE-003.

In some embodiments, the present disclosure pertains to: A method of treatment of Huntington's disease in a subject in need thereof, wherein the subject is administered a HTT oligonucleotide which is formulated as a liquid formulation, wherein the liquid formulation is reconstituted from a lyophilized preparation with a solution of sodium chloride.

In some embodiments, a lyophilized preparation of WVE-003 is a dry powder.

In some embodiments, a lyophilized preparation of WVE-003 is a dry powder prepared by lyophilization of a liquid formulation of WVE-003 in water.

In some embodiments, a lyophilized preparation of WVE-003 is a dry powder in a vial.

In some embodiments, a lyophilized preparation of WVE-003 is about 20 mg of a dry powder in a vial.

In some embodiments, a lyophilized preparation of WVE-003 is a dry powder in a 10 ml vial.

In some embodiments, a lyophilized preparation of WVE-003 is about 20 mg of a dry powder in a 10 ml vial.

The reconstituted solution does not contain any preservatives; therefore, it should be administered without delay. If this is not possible, the solution should be stored at room temperature and administered within 4 hours.

Partially used, unused, or damaged vials should be disposed of.

In some embodiments, HTT oligonucleotides WVE-003 were analyzed in nonclinical and clinical studies.

In some embodiments, WVE-003 is provided in a solution. In some embodiments, WVE-003 is administered in a solution. In some embodiments, WVE-003 is dissolved in a suitable diluent. In some embodiments, a diluent is aCSF. In some embodiments, a WVE-003 composition is a WVE-003 preparation (e.g., drug substance, drug product, etc.) dissolved in aCSF.

### Dosing Regimen of a HTT Oligonucleotide

In some embodiments, a dosing regimen of an oligonucleotide, an oligonucleotide composition, a chirally controlled oligonucleotide composition, or a therapeutically effective amount of any thereof, is any dosing regimen described herein.

In some embodiments, a dosing regimen pertains to: the amount of an individual dose of an oligonucleotide, an oligonucleotide composition, a chirally controlled oligonucleotide composition, or a therapeutically effective amount of an oligonucleotide, an oligonucleotide composition, a chirally controlled oligonucleotide composition; and/or the interval between multiple or successive doses thereof, and/or the total length or duration of time during which a subject receives one or more doses thereof; and/or a particular formulation thereof.

In some embodiments, a dosing regimen of WVE-003, an oligonucleotide composition of WVE-003, a chirally controlled oligonucleotide composition of WVE-003, or a therapeutically effective amount of WVE-003, an oligonucleotide composition of WVE-003, a chirally controlled oligonucleotide composition of WVE-003, or a therapeutically effective amount of an oligonucleotide composition of WVE-003, a chirally controlled oligonucleotide composition of WVE-003, or a therapeutically effective amount of WVE-003, an oligonucleotide composition of WVE-003, a chirally controlled oligonucleotide composition of WVE-003 is any dosing regimen described herein. In some embodiments, a dosing regimen includes but is not limited to a specific amount (e.g., about 30 mg, about 60 mg, about 90 mg, about 120 mg, about 150 mg, and/or about 168 mg per dose, per dosage, and/or per administration) and/or particular dosing intervals (e.g., once about every 4 weeks, once about every 8 weeks, once about every 12 weeks, once about every month, once about every 2 months, etc.), and/or particular lengths or durations of dosing (e.g., dosing occurs over a span of time of at least: about 1 month, about 2 month, about 3 months, about 4 months, about 5 months, about 6 months, about 7 months, about 8 months, about 9 months, about 10 months, about 11 months, about 12 months, about 18 months, about 24 months, about 36 months, about 48 months, and/or at least: about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52, about 78 weeks, about 104 weeks, about 156 weeks, about 208 weeks, etc.). In some embodiments, a dosing regimen is any dosing regimen described herein. In some embodiments, a dosing regimen is or comprises a dosing regimen described in the Examples.

In some embodiments, a dose of WVE-003 (e.g., about 30, about 60, about 90, about 120, about 150 or about 168 mg) is administered to a subject in need thereof, on a regular schedule or dosing regimen.

In some embodiments, a dose of WVE-003 is administered approximately monthly. In some embodiments, a dose of WVE-003 is administered approximately once every 2 months. In some embodiments, a dose of WVE-003 is administered approximately once every 8 weeks. In some embodiments, a dose of WVE-003 is administered approximately once every 12 weeks.

In some embodiments, a dose of WVE-003 is administered approximately monthly for at least about three months. In some embodiments, a dose of WVE-003 is administered approximately once every 2 months for at least about four months. In some embodiments, a dose of WVE-003 is administered approximately once every 8 weeks for at least about 16 weeks. In some embodiments, a dose of WVE-003 is administered approximately once every 12 weeks for at least about 12 weeks.

In some embodiments, a dose of WVE-003 is administered approximately monthly, and preceding the first monthly dose, WVE-003 is administered, followed by an approximately 8-week (approximately 2-month) washout period. In some embodiments, a dose of WVE-003 is administered approximately once every 2 months, and preceding the first once every 2 months dose, WVE-003 is administered, followed by an approximately 12-week (approximately 3-month) washout period. In some embodiments, a dose of WVE-003 is administered approximately once every 8 weeks, and preceding the first once every 8 weeks dose, WVE-003 is administered, followed by an approximately 12-week (approximately 3-month) washout period.

In some embodiments, a dose of WVE-003 is administered, followed by an approximately 8-week (approximately 2-month) washout period, followed by approximately monthly doses of WVE-003. In some embodiments, a dose of WVE-003 is administered, followed by an approximately 12-week (approximately 3-month) washout period, followed by approximately once every 2 months doses of WVE-003. In some embodiments, a dose of WVE-003 is administered, followed by an approximately 12-week (approximately 3-month) washout period, followed by approximately once every 8 weeks doses of WVE-003.

In some embodiments, a lumbar puncture (spinal tap) procedure is performed to obtain CSF for analysis after one or more doses of the oligonucleotide.

In some embodiments, a lumbar puncture (spinal tap) procedure is performed to obtain CSF for analysis after approximately 1 month (approximately 4 weeks) after the third monthly dose.

### Certain Methods of Use of a HTT Oligonucleotide or HTT Oligonucleotide Composition

In some embodiments, the present disclosure provides a method for treating or preventing Huntington's disease, wherein the method comprises the step of administering to a subject an effective amount of a HTT oligonucleotide or composition thereof described herein (e.g., WVE-003).

In some embodiments, a HTT transcript is of Huntingtin gene or a variant thereof.

In some embodiments, HTT oligonucleotides can elicit pro-inflammatory responses. In some embodiments, the present disclosure provides compositions and methods for reducing inflammation. In some embodiments, the present disclosure provides compositions and methods for reducing pro-inflammatory responses. In some embodiments, the present disclosure provides methods for reducing injection site inflammation using provided compositions. In some embodiments, the present disclosure provides methods for reducing drug-induced vascular injury using provided compositions.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 10 to about 168 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of about 30 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of about 60 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of about 90 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of about 120 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of about 150 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of about 168 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 60 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 90 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 120 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 150 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 168 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 30 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 60 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 90 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 120 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 150 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 168 mg approximately monthly, such that disease progression in the subject is delayed, thereby treating the subject, wherein the HTT oligonucleotide or HTT oligonucleotide is capable of mediating a decrease in the level, expression and/or activity of a mHTT transcript.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 30 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 60 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 90 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 120 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 150 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 168 mg, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 30 mg±5%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 60 mg±5%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 90 mg±5%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 120 mg±5%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 150 mg±5%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 168 mg±5%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 30 mg±10%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 60 mg±10%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 90 mg±10%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 120 mg±10%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 150 mg±10%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 168 mg±10%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 30 mg±15%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 60 mg±15%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 90 mg±15%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 120 mg±15%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 150 mg±15%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 168 mg±15%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 30 mg±20%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 60 mg±20%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 90 mg±25%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 120 mg±25%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 150 mg±25%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 168 mg±25%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 30 mg±30%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 60 mg±30%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 90 mg±30%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 120 mg±30%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 150 mg±30%, such that progression of Huntington's disease in the subject is delayed, and/or the onset of Huntington's disease is delayed, and/or the severity of a symptom of Huntington's disease is reduced.

In some embodiments, the present disclosure pertains to: A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of 168 mg±30%, such that progression of Huntington's disease in the subject is delayed, and/or the onset of Huntington's disease is delayed, and/or the severity of a symptom of Huntington's disease is reduced.

In some embodiments, the subject is administered a steroid prior to the first dose of a chirally controlled HTT oligonucleotide composition.

In some embodiments, the subject is administered a steroid at least about one month prior to the first dose of a HTT oligonucleotide or a HTT oligonucleotide composition.

In some embodiments, the subject is on a steroid regimen for at least about one month prior to the first dose of a HTT oligonucleotide or a HTT oligonucleotide composition.

In some embodiments, the subject is administered an oral corticosteroid for at least about 24 months prior to the first dose of a HTT oligonucleotide or a HTT oligonucleotide composition.

In some embodiments, the subject is administered hydrocortisone and/or acetaminophen within 24 hours of administration of a HTT oligonucleotide or a HTT oligonucleotide composition.

In some embodiments, the subject is administered a HTT oligonucleotide or a HTT oligonucleotide composition approximately monthly for at least about 2 months.

In some embodiments, the subject is administered a HTT oligonucleotide or a HTT oligonucleotide composition approximately monthly for at least about 4 months.

In some embodiments, the subject is administered a HTT oligonucleotide or a HTT oligonucleotide composition approximately monthly for at least about 8 months.

In some embodiments, the subject is administered a HTT oligonucleotide or a HTT oligonucleotide composition approximately monthly for at least about 12 months.

In some embodiments, the subject is administered a HTT oligonucleotide or a HTT oligonucleotide composition approximately monthly for at least about 16 months.

In some embodiments, the subject is administered a HTT oligonucleotide or a HTT oligonucleotide composition approximately monthly for at least about 32 months.

In some embodiments, the subject is administered a HTT oligonucleotide or a HTT oligonucleotide composition approximately monthly for at least about 48 months.

In some embodiments, the subject is administered a HTT oligonucleotide or a HTT oligonucleotide composition approximately monthly for at least about 50 months.

In some embodiments, the subject is administered a HTT oligonucleotide or a HTT oligonucleotide composition approximately monthly for at least about 60 months.

In some embodiments, the subject is administered a HTT oligonucleotide or a HTT oligonucleotide composition approximately monthly for at least about 70 months.

In some embodiments, the subject is administered a HTT oligonucleotide or a HTT oligonucleotide composition approximately monthly for at least about 80 months.

In some embodiments, the subject is administered a HTT oligonucleotide or a HTT oligonucleotide composition approximately monthly for at least about 90 months.

In some embodiments, the subject is administered a HTT oligonucleotide or a HTT oligonucleotide composition approximately monthly for at least about 100 months.

In some embodiments, the present disclosure pertains to: A method for reducing the severity of and/or delaying the onset of at least one symptom of Huntington's disease in a subject with Huntington's disease in need thereof who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 10 to about 168 mg approximately monthly, thereby reducing the severity of and/or delaying the onset of at least one symptom of Huntington's disease relative to baseline, in the subject, wherein the HTT oligonucleotide or HTT oligonucleotide is capable of mediating a decrease in the level, expression and/or activity of a mHTT transcript.

In some embodiments, the Huntingtin protein production is measured by reverse transcription polymerase chain reaction (RT-PCR), Western blot analysis, or immunohistochemical detection.

In some embodiments, the present disclosure pertains to: A method for treating a subject with Huntington's disease in need thereof who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 10 to about 168 mg approximately monthly, such that disease progression in the subject is delayed (e.g., as measured by any appropriate technique described herein or known in the art), thereby treating the subject, wherein the HTT oligonucleotide or HTT oligonucleotide is capable of mediating a decrease in the level, expression and/or activity of a mHTT transcript.

In some embodiments, the present disclosure pertains to: A method for reducing the severity of and/or delaying the onset of at least one symptom of Huntington's disease in a subject with Huntington's disease in need thereof who has a mutation of the HTT gene that is amenable to an allele-specific decrease in the level, expression and/or activity of mHTT, comprising intrathecally administering to the subject a HTT oligonucleotide or a HTT oligonucleotide composition at a dose of 10 to about 168 mg approximately monthly, thereby reducing the severity of and/or delaying the onset of at least one symptom of Huntington's disease relative to baseline in the subject as measured by any method known in the art and/or described herein, wherein the HTT oligonucleotide or HTT oligonucleotide is capable of mediating a decrease in the level, expression and/or activity of a mHTT transcript.

In some embodiments, the method further comprises the step of administering to the subject a corticosteroid.

In some embodiments, the corticosteroid is Betamethasone, Budesonide, Cortisone, Dexamethasone. Hydrocortisone, Methylprednisolone, Prednisolone, or Prednisone.

In some embodiments, the corticosteroid is administered prior to, in conjunction with, or subsequent to administration of a HTT oligonucleotide or a HTT oligonucleotide composition.

In some embodiments, the method further comprises the step of confirming that the subject has a mutation in the HTT gene that is amenable to an allele-specific decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of 10 to about 168 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of 10 to about 168 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of about 30 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of about 60 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of about 90 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of about 120 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of about 150 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject a chirally controlled HTT oligonucleotide composition at a dose of about 168 mg, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of 10 to about 168 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of about 30 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of about 60 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of about 90 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of about 120 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of about 150 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject a chirally controlled HTT oligonucleotide composition at a dose of about 168 mg approximately monthly, such that disease progression in the subject is delayed, thereby treating the subject, wherein the chirally controlled HTT oligonucleotide composition is capable of mediating a decrease in the level, expression and/or activity of a mHTT transcript.

In some embodiments, the intrathecal administration is a bolus.

In some embodiments, the subject is administered a steroid prior to the first dose of a chirally controlled HTT oligonucleotide composition.

In some embodiments, the subject is administered a steroid at least about one month prior to the first dose of a chirally controlled HTT oligonucleotide composition.

In some embodiments, the subject is on a steroid regimen for at least about one month prior to the first dose of a chirally controlled HTT oligonucleotide composition.

In some embodiments, the subject is administered an oral corticosteroid for at least about 24 months prior to the first dose of a chirally controlled HTT oligonucleotide composition.

In some embodiments, the subject is administered hydrocortisone and/or acetaminophen within 24 hours of administration of a chirally controlled HTT oligonucleotide composition.

In some embodiments, the subject is administered a chirally controlled HTT oligonucleotide composition approximately monthly for at least about 2 months.

In some embodiments, the subject is administered a chirally controlled HTT oligonucleotide composition approximately monthly for at least about 4 months.

In some embodiments, the subject is administered a chirally controlled HTT oligonucleotide composition approximately monthly for at least about 8 months.

In some embodiments, the subject is administered a chirally controlled HTT oligonucleotide composition approximately monthly for at least about 12 months.

In some embodiments, the subject is administered a chirally controlled HTT oligonucleotide composition approximately monthly for at least about 16 months.

In some embodiments, the subject is administered a chirally controlled HTT oligonucleotide composition approximately monthly for at least about 32 months.

In some embodiments, the subject is administered a chirally controlled HTT oligonucleotide composition approximately monthly for at least about 48 months.

In some embodiments, the subject is administered a chirally controlled HTT oligonucleotide composition approximately monthly for at least about 50 months.

In some embodiments, the subject is administered a chirally controlled HTT oligonucleotide composition approximately monthly for at least about 60 months.

In some embodiments, the subject is administered a chirally controlled HTT oligonucleotide composition approximately monthly for at least about 70 months.

In some embodiments, the subject is administered a chirally controlled HTT oligonucleotide composition approximately monthly for at least about 80 months.

In some embodiments, the subject is administered a chirally controlled HTT oligonucleotide composition approximately monthly for at least about 90 months.

In some embodiments, the subject is administered a chirally controlled HTT oligonucleotide composition approximately monthly for at least about 100 months.

In some embodiments, the present disclosure pertains to: A method for reducing the severity of and/or delaying the onset of at least one symptom of Huntington's disease in a subject with Huntington's disease in need thereof who has a mutation of the HTT gene that is amenable to an allele-specific decrease in the level, expression and/or activity of mHTT, comprising intrathecally administering to the subject a chirally controlled HTT oligonucleotide composition at a dose of 10 to about 168 mg approximately monthly, thereby reducing the severity of and/or delaying the onset of at least one symptom of Huntington's disease relative to baseline, in the subject, wherein the chirally controlled HTT oligonucleotide composition is capable of mediating a decrease in the level, expression and/or activity of a mHTT transcript.

In some embodiments, the Huntingtin protein production is measured by reverse transcription polymerase chain reaction (RT-PCR), Western blot analysis, or immunohistochemical detection.

In some embodiments, the present disclosure pertains to: A method for treating a subject with Huntington's disease in need thereof who has a mutation of the HTT gene that is amenable to an allele-specific decrease in the level, expression and/or activity of mHTT, comprising intrathecally administering to the subject a chirally controlled HTT oligonucleotide composition at a dose of 10 to about 168 mg approximately monthly, such that disease progression in the subject is delayed as measured by a protocol described herein (e.g., in the Examples), thereby treating the subject, wherein the chirally controlled HTT oligonucleotide composition is capable of mediating a decrease in the level, expression and/or activity of a mHTT transcript.

In some embodiments, the present disclosure pertains to: A method for reducing the severity of and/or delaying the onset of at least one symptom of Huntington's disease in a subject with Huntington's disease in need thereof who has a mutation of the HTT gene that is amenable to an allele-specific decrease in the level, expression and/or activity of mHTT, comprising intrathecally administering to the subject a chirally controlled HTT oligonucleotide composition at a dose of 10 to about 168 mg approximately monthly, thereby reducing the severity of and/or delaying the onset of at least one symptom of Huntington's disease relative to baseline in the subject, wherein the chirally controlled HTT oligonucleotide composition is capable of mediating a decrease in the level, expression and/or activity of a mHTT transcript.

In some embodiments, the intrathecal administration is a bolus.

In some embodiments, the method further comprises the step of administering to the subject a corticosteroid.

In some embodiments, the corticosteroid is Betamethasone, Budesonide, Cortisone, Dexamethasone. Hydrocortisone, Methylprednisolone, Prednisolone, or Prednisone.

In some embodiments, the corticosteroid is administered prior to, in conjunction with, or subsequent to administration of a chirally controlled HTT oligonucleotide composition.

In some embodiments, the method further comprises the step of confirming that the subject has a mutation in the HTT gene that is amenable to an allele-specific decrease in the level, expression and/or activity of a mHTT transcript.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of 10 to about 168 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of 10 to about 168 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of 10 to 168 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of 10 to about 168 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of about 30 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of about 60 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of about 90 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of about 120 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of about 150 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising the step of administering to the subject WVE-003 (or a salt form thereof) at a dose of about 168 mg, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of 10 to about 168 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of 10 to about 168 approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of 30, 60, 90, 120, 150, or 168 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of about 30 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of about 60 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of about 90 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of about 120 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of about 150 mg approximately monthly, such that disease progression in the subject is delayed, thereby treating the subject.

In some embodiments, the present disclosure pertains to: A method for treating a subject with Huntington's disease who has a mutation of the HTT gene that is amenable to an allele-specific decrease in the level, expression and/or activity of mHTT, comprising intrathecally administering to the subject WVE-003 (or a salt form thereof) at a dose of about 168 mg approximately monthly, such that disease progression in the subject is delayed, thereby treating the subject.

In some embodiments, the subject is administered a steroid prior to the first dose of WVE-003.

In some embodiments, the subject is administered a steroid at least about one month prior to the first dose of WVE-003.

In some embodiments, the subject is on a steroid regimen for at least about one month prior to the first dose of WVE-003.

In some embodiments, the subject is administered an oral corticosteroid for at least about 24 months prior to the first dose of WVE-003.

In some embodiments, the subject is administered hydrocortisone and/or acetaminophen within 24 hours of administration of WVE-003.

In some embodiments, the subject is administered WVE-003 approximately monthly for at least about 2 months.

In some embodiments, the subject is administered WVE-003 approximately monthly for at least about 4 months.

In some embodiments, the subject is administered WVE-003 approximately monthly for at least about 8 months.

In some embodiments, the subject is administered WVE-003 approximately monthly for at least about 12 months.

In some embodiments, the subject is administered WVE-003 approximately monthly for at least about 16 months.

In some embodiments, the subject is administered WVE-003 approximately monthly for at least about 32 months.

In some embodiments, the subject is administered WVE-003 approximately monthly for at least about 48 months.

In some embodiments, the subject is administered WVE-003 approximately monthly for at least about 50 months.

In some embodiments, the subject is administered WVE-003 approximately monthly for at least about 60 months.

In some embodiments, the subject is administered WVE-003 approximately monthly for at least about 70 months.

In some embodiments, the subject is administered WVE-003 approximately monthly for at least about 80 months.

In some embodiments, the subject is administered WVE-003 approximately monthly for at least about 90 months.

In some embodiments, the subject is administered WVE-003 approximately monthly for at least about 100 months.

In some embodiments, the present disclosure pertains to: A method for reducing the severity of and/or delaying the onset of at least one symptom of Huntington's disease in a subject with Huntington's disease in need thereof who has a mutation of the HTT gene that is amenable to an allele-specific decrease in the level, expression and/or activity of mHTT, comprising intrathecally administering to the subject WVE-003 (or a salt form thereof) at a dose of 10 to about 168 mg approximately monthly, thereby reducing the severity of and/or delaying the onset of at least one symptom of Huntington's disease relative to baseline, in the subject.

In some embodiments, the Huntingtin protein production is measured by reverse transcription polymerase chain reaction (RT-PCR), Western blot analysis, or immunohistochemical detection.

In some embodiments, the method further comprises the step of administering to the subject a corticosteroid.

In some embodiments, the corticosteroid is Betamethasone, Budesonide, Cortisone, Dexamethasone. Hydrocortisone, Methylprednisolone, Prednisolone, or Prednisone.

In some embodiments, the corticosteroid is administered prior to, in conjunction with, or subsequent to administration of WVE-003.

In some embodiments, the method further comprises the step of confirming that the subject has a mutation in the HTT gene that is amenable to an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of 10 to about 168 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of 30, 60, 90, 120, 150, or 168 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 30 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 60 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 90 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 120 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 150 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 168 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 30 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 60 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising the step of administering to the subject a chirally controlled HTT oligonucleotide composition at a dose of about 90 mg, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 120 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 150 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 168 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 30 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 60 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 90 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 120 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 150 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 168 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 30 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 60 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 90 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 120 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 150 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the present disclosure pertains to: A method for treating a subject with Huntington's disease who has a mutation of the HTT gene that is amenable to allele-specific knockdown of a mutant HTT transcript, comprising intrathecally administering to the subject a chirally controlled HTT oligonucleotide composition at a dose of about 168 mg approximately monthly, such that disease progression in the subject is delayed, thereby treating the subject, wherein the chirally controlled HTT oligonucleotide composition is capable of mediating an allele-specific decrease in the level, expression and/or activity of a mHTT transcript.

In some embodiments, the subject is administered a steroid prior to the first dose of a chirally controlled HTT oligonucleotide composition.

In some embodiments, the subject is administered a steroid at least about one month prior to the first dose of a chirally controlled HTT oligonucleotide composition.

In some embodiments, the subject is on a steroid regimen for at least about one month prior to the first dose of a chirally controlled HTT oligonucleotide composition.

In some embodiments, the subject is administered an oral corticosteroid for at least about 24 months prior to the first dose of a chirally controlled HTT oligonucleotide composition.

In some embodiments, the subject is administered hydrocortisone and/or acetaminophen within 24 hours of administration of a chirally controlled HTT oligonucleotide composition.

In some embodiments, the subject is administered a chirally controlled HTT oligonucleotide composition approximately monthly for at least about 2 months.

In some embodiments, the subject is administered a chirally controlled HTT oligonucleotide composition approximately monthly for at least about 4 months.

In some embodiments, the subject is administered a chirally controlled HTT oligonucleotide composition approximately monthly for at least about 8 months.

In some embodiments, the subject is administered a chirally controlled HTT oligonucleotide composition approximately monthly for at least about 12 months.

In some embodiments, the subject is administered a chirally controlled HTT oligonucleotide composition approximately monthly for at least about 16 months.

In some embodiments, the subject is administered a chirally controlled HTT oligonucleotide composition approximately monthly for at least about 32 months.

In some embodiments, the subject is administered a chirally controlled HTT oligonucleotide composition approximately monthly for at least about 48 months.

In some embodiments, the subject is administered a chirally controlled HTT oligonucleotide composition approximately monthly for at least about 50 months.

In some embodiments, the subject is administered a chirally controlled HTT oligonucleotide composition approximately monthly for at least about 60 months.

In some embodiments, the subject is administered a chirally controlled HTT oligonucleotide composition approximately monthly for at least about 70 months.

In some embodiments, the subject is administered a chirally controlled HTT oligonucleotide composition approximately monthly for at least about 80 months.

In some embodiments, the subject is administered a chirally controlled HTT oligonucleotide composition approximately monthly for at least about 90 months.

In some embodiments, the subject is administered a chirally controlled HTT oligonucleotide composition approximately monthly for at least about 100 months.

In some embodiments, the present disclosure pertains to: A method for reducing the severity of and/or delaying the onset of at least one symptom of Huntington's disease in a subject with Huntington's disease in need thereof who has a mutation of the HTT gene that is amenable to allele-specific knockdown of a mutant HTT transcript, comprising intrathecally administering to the subject a chirally controlled HTT oligonucleotide composition at a dose of 10 to about 168 mg approximately monthly, thereby reducing the severity of and/or delaying the onset of at least one symptom of Huntington's disease relative to baseline, in the subject, wherein the chirally controlled HTT oligonucleotide composition is capable of mediating an allele-specific decrease in the level, expression and/or activity of a mHTT transcript.

In some embodiments, the Huntingtin protein production is measured by reverse transcription polymerase chain reaction (RT-PCR), Western blot analysis, or immunohistochemical detection.

In some embodiments, the method further comprises the step of administering to the subject a corticosteroid.

In some embodiments, the corticosteroid is Betamethasone, Budesonide, Cortisone, Dexamethasone, Hydrocortisone, Methylprednisolone, Prednisolone, or Prednisone.

In some embodiments, the corticosteroid is administered prior to, in conjunction with, or subsequent to administration of a chirally controlled HTT oligonucleotide composition.

In some embodiments, the method further comprises the step of confirming that the subject has a mutation in the HTT gene that is amenable to allele-specific knockdown of a mutant HTT transcript.

In some embodiments, the present disclosure provides methods of use of a composition comprising any HTT oligonucleotide disclosed herein. In some embodiments, the present disclosure provides methods of use of a composition comprising any chirally controlled HTT oligonucleotide disclosed herein.

In some embodiments, the present disclosure provides methods of use of a composition comprising a HTT oligonucleotide disclosed herein which is capable of mediating allele-specific knockdown of mutant HTT. In some embodiments, the present disclosure provides methods of use of a composition comprising a HTT oligonucleotide disclosed herein which is capable of mediating allele-specific knockdown of a mutant HTT transcript. In some embodiments, the present disclosure provides methods of use of a composition comprising a HTT oligonucleotide disclosed herein which is capable of mediating allele-specific knockdown of a mutant HTT transcript. In some embodiments, the present disclosure provides methods of use of a composition comprising a HTT oligonucleotide(s) disclosed herein which is capable of mediating allele-specific knockdown of mutant HTT transcript. In some embodiments, such a composition is a chirally controlled HTT oligonucleotide composition.

In some embodiments, the HTT oligonucleotide is WVE-003.

In some embodiments, the present disclosure provides a pharmaceutical composition comprising a HTT oligonucleotide or a HTT oligonucleotide composition of the present disclosure (e.g., WVE-003) and a pharmaceutically acceptable carrier (including but not limited to, a pharmaceutically acceptable salt).

In some embodiments, the present disclosure provides a method for allele-specific knockdown of a target HTT transcript, comprising the step of administering a HTT oligonucleotide composition of the present disclosure. In some embodiments, the present disclosure provides a method for reducing level of a HTT transcript or a product thereof, comprising the step of administering a HTT oligonucleotide composition of the present disclosure. A method for treating Huntington's disease, comprising the step of administering to a subject susceptible thereto or suffering therefrom a composition described in the present disclosure.

In some embodiments, the present disclosure provides a method for treating Huntington's disease, comprising the step of administering to a subject susceptible thereto or suffering therefrom a composition comprising any HTT oligonucleotide disclosed herein.

In some embodiments, the present disclosure provides a method for treating Huntington's disease, comprising (a) administering to a subject susceptible thereto or suffering therefrom a composition comprising any HTT oligonucleotide disclosed herein, and (b) administering to the subject additional treatment which is capable of preventing, treating, ameliorating or slowing the progress of Huntington's disease.

Certain abbreviates are listed below:
aCSF: artificial cerebrospinal fluid
aPTT: activated partial thromboplastin time
ASO: antisense oligonucleotide
AUC: area under the plasma concentration-time curve
AUC0-24: area under the curve from time 0 to 24 hours
AUClast: area under the curve from 0 to time of the last quantifiable concentration
BLQ, BLOQ: below the limit of quantification
BP: British Pharmacopeia
BUN: blood urea nitrogen
C variant: wild-type variant of SNP1
C0: extrapolated initial concentration at time 0
CAG: cytosine-adenine-guanine
Cmax: maximum observed concentration
CNS: central nervous system
CSF: cerebrospinal fluid
CYP: cytochrome P450
DNA: deoxyribonucleic acid
ECG: electrocardiogram
F: Female
FOB: functional observational battery
GAPDH: glyceraldehyde 3-phosphate dehydrogenase
GLP: Good Laboratory Practice
HD: Huntington's disease
HED: human equivalent dose
HLGT: high level group term
HPBL: human peripheral blood lymphocytes
HTT, HTT: huntingtin, Huntingtin
INN: International Nonproprietary Name
ISH: in situ hybridization
IT: Intrathecal
IV: Intravenous
KD: knockdown
KO: knock out
LC-MS/MS: liquid chromatography-tandem mass spectrometry
LFT: liver function test
LLOQ: lower limit of quantification
M: male
MedDRA: Medical Dictionary for Regulatory Activities
mHTT, mHTT: mutant huntingtin protein, mutant Huntingtin gene
mRNA: messenger ribonucleic acid
N: number
NA: not applicable
NOAEL: no-observed-adverse-effect level
NOEL: no-observed-adverse-effect level
NOEL: no-observed-effect level
NPSR1: Neuropeptide S Receptor 1
NT, nt: Nucleotide
OECD: Organisation for Economic Cooperation and Development
PBMC: peripheral blood mononuclear cell
PCR: polymerase chain reaction
PD: pharmacodynamics
PK: pharmacokinetics
PS: phosphorothioate
PT: preferred term
q4w: administered every 4 weeks
q4w: every 4 weeks
RNA: ribonucleic acid
RNase HC: Catalytic domain of human RNase H enzyme
SAE: serious adverse event
SNP: single nucleotide polymorphism
SNP1: single nucleotide polymorphism (e.g., rs362273)
t1/2: terminal elimination half-life
TEAE: treatment-emergent adverse event
TK: toxicokinetic
TLR9: toll-like receptor 9
tmax: time of occurrence of Cmax
U variant: mutant variant of SNP2
USP: United States Pharmacopoeia
UV: ultraviolet
VC: vehicle control
wtHTT/wtHTT: wild type huntingtin protein/wild type Huntingtin gene

Among other things, the present disclosure provides the following Example Embodiments:

1. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, about 60 mg, about 90 mg, about 120 mg, about 150 mg, about 160 mg, or about 168 mg, such that progression of Huntington's disease in the subject is delayed, and/or the onset of Huntington's disease is delayed, and/or the severity of a symptom of Huntington's disease is reduced.

2. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, such that progression of Huntington's disease in the subject is delayed, and/or the onset of Huntington's disease is delayed, and/or the severity of a symptom of Huntington's disease is reduced.

3. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 60 mg, such that progression of Huntington's disease in the subject is delayed, and/or the onset of Huntington's disease is delayed, and/or the severity of a symptom of Huntington's disease is reduced.

4. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 90 mg, such that progression of Huntington's disease in the subject is delayed, and/or the onset of Huntington's disease is delayed, and/or the severity of a symptom of Huntington's disease is reduced.

5. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 120 mg, such that progression of Huntington's disease in the subject is delayed, and/or the onset of Huntington's disease is delayed, and/or the severity of a symptom of Huntington's disease is reduced.

6. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 150 mg, such that progression of Huntington's disease in the subject is delayed, and/or the onset of Huntington's disease is delayed, and/or the severity of a symptom of Huntington's disease is reduced.

7. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 160 mg, such that progression of Huntington's disease in the subject is delayed, and/or the onset of Huntington's disease is delayed, and/or the severity of a symptom of Huntington's disease is reduced.

8. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 168 mg, such that progression of Huntington's disease in the subject is delayed, and/or the onset of Huntington's disease is delayed, and/or the severity of a symptom of Huntington's disease is reduced.

9. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, such that disease progression in the subject is delayed, and/or the onset of Huntington's disease is delayed, and/or the severity of a symptom of Huntington's disease is reduced.

10. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 60 mg, such that disease progression in the subject is delayed, and/or the onset of Huntington's disease is delayed, and/or the severity of a symptom of Huntington's disease is reduced.

11. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 90 mg, such that disease progression in the subject is delayed, and/or the onset of Huntington's disease is delayed, and/or the severity of a symptom of Huntington's disease is reduced.

12. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 120 mg, such that disease progression in the subject is delayed, and/or the onset of Huntington's disease is delayed, and/or the severity of a symptom of Huntington's disease is reduced.

13. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 150 mg, such that disease progression in the subject is delayed, and/or the onset of Huntington's disease is delayed, and/or the severity of a symptom of Huntington's disease is reduced.

14. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 160 mg, such that disease progression in the subject is delayed, and/or the onset of Huntington's disease is delayed, and/or the severity of a symptom of Huntington's disease is reduced.

15. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 168 mg, such that disease progression in the subject is delayed, and/or the onset of Huntington's disease is delayed, and/or the severity of a symptom of Huntington's disease is reduced.

16. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 30 mg.

17. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 60 mg.

18. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 90 mg.

19. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 120 mg.

20. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 150 mg.

21. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 160 mg.

22. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 168 mg.

23. A method for treating Huntington's disease, comprising administering to a subject suffering therefrom WVE-003 (or a salt form thereof), wherein WVE-003 is administered at a dose of about 30 mg, about 60 mg, about 90 mg, about 120 mg, about 150 mg, about 160 mg, or about 168 mg, and wherein the subject has a HTT allele that comprises an expanded CAG repeat region and is fully complementary to the base sequence of WVE-003.

24. A method for treating Huntington's disease, comprising administering to a subject suffering therefrom a pharmaceutical composition that comprises or delivers WVE-003 (or a salt form thereof), wherein WVE-003 is administered at a dose of about 30 mg, about 60 mg, about 90 mg, about 120 mg, about 150 mg, about 160 mg, or about 168 mg, and wherein the subject has a HTT allele that comprises an expanded CAG repeat region and is fully complementary to the base sequence of WVE-003.

25. A method, comprising administering to a subject WVE-003 (or a salt form thereof), wherein the subject is determined to have a genetic sequence that is the same or fully complementary to the base sequence of WVE-003.

26. A method, comprising administering to a subject WVE-003 (or a salt form thereof), wherein the subject is determined to have a genetic sequence that comprises a sequence that is the same or fully complementary to the base sequence of WVE-003 and a sequence that is or encodes an expanded CAG repeats.

27. A method, comprising administering to a subject WVE-003 (or a salt form thereof), wherein the subject is determined to have a genetic sequence that encodes a transcript that comprises an expanded CAG repeat in HTT and is fully complementary to the base sequence of WVE-003.

28. A method, comprising administering to a subject WVE-003 (or a salt form thereof), wherein the subject is determined to express a HTT transcript that comprises an expanded CAG repeat and is fully complementary to the base sequence of WVE-003.

29. The method of any one of Embodiments 25-28, wherein the subject is determined to have a genetic sequence or transcript that is not the same or fully complementary to the base sequence of WVE-003 at rs362273.

30. The method of any one of Embodiments 25-29, wherein the subject is determined to have a genetic sequence or transcript that is not the same or fully complementary to the base sequence of WVE-003 at rs362273 and that does not contain expanded CAG repeats (or a sequence encoded thereby).

31. The method of any one of Embodiments 25-30, wherein WVE-003 is administered at a dose of about 30 mg.

32. The method of any one of Embodiments 25-30, wherein WVE-003 is administered at a dose of about 60 mg.

33. The method of any one of Embodiments 25-30, wherein WVE-003 is administered at a dose of about 90 mg.

34. The method of any one of Embodiments 25-30, wherein WVE-003 is administered at a dose of about 120 mg.

35. The method of any one of Embodiments 25-30, wherein WVE-003 is administered at a dose of about 150 mg.

36. The method of any one of Embodiments 25-30, wherein WVE-003 is administered at a dose of about 168 mg.

37. The method of any one of Embodiments 25-36, wherein two or more doses are administered.

38. The method of any one of Embodiments 25-37, wherein a dose is administered about one week after an immediate preceding dose.

39. The method of any one of Embodiments 25-38, wherein a dose is administered about two weeks after an immediate preceding dose.

40. The method of any one of Embodiments 25-39, wherein a dose is administered about four weeks after an immediate preceding dose.

41. The method of any one of Embodiments 25-40, wherein a dose is administered about eight weeks after an immediate preceding dose.

42. The method of any one of Embodiments 25-41, wherein a dose is administered about twelve weeks after an immediate preceding dose.

43. The method of any one of Embodiments 25-42, wherein a dose is administered about one month after an immediate preceding dose.

44. The method of any one of Embodiments 25-43, wherein a dose is administered about two months after an immediate preceding dose.

45. The method of any one of Embodiments 25-44, wherein a dose is administered about 3, 4, 5, 6 or more months after an immediate preceding dose.

46. The method of any one of Embodiments 25-37, comprising administering multiple doses weekly.

47. The method of any one of Embodiments 25-37, comprising administering multiple doses every two weeks.

48. The method of any one of Embodiments 25-37, comprising administering multiple doses about every four weeks.

49. The methods of any one of Embodiments 25-37, comprising administering multiple doses about every eight weeks.

50. The methods of any one of Embodiments 25-37, comprising administering multiple doses about every 12 weeks.

51. The method of any one of Embodiments 25-37, comprising administering multiple doses monthly.

52. The method of any one of Embodiments 25-37, comprising administering multiple doses every two months.

53. The method of any one of Embodiments 25-37, comprising administering multiple doses every 3, 4, 5, 6 or more months.

54. The method of any one of Embodiments 25-53, wherein expression of mutant HTT is reduced.

55. The method of any one of Embodiments 25-54, wherein mutant HTT protein level is reduced.

56. The method of any one of Embodiments 54-55, wherein the reduction is about 10% or more.

57. The method of any one of Embodiments 54-55, wherein the reduction is about 12% or more.

58. The method of any one of Embodiments 54-55, wherein the reduction is about 15% or more.

59. The method of any one of Embodiments 54-55, wherein the reduction is about 20%, 25%, 30%, 40%, 50%, 60%, 70%, 80%, or 90% or more.

60. The method of any one of Embodiments 54-59, wherein the reduction is assessed for an individual subject.

61. The method of any one of Embodiments 54-59, wherein the reduction is assessed for a population of subjects.

62. The method of Embodiment 61, wherein the reduction is assessed for a population of subjects, wherein the population size is about 10, 20, 30, 40, 50, 60, 70, 80, 90, 100, 200, 500, 1000 or more subjects.

63. The method of Embodiment 60 or 61, wherein subjects in the population receive the same dosage regimen.

64. The method of Embodiment 60 or 61, wherein subjects in the population receive different dosage regimens.

65. The method of any one of Embodiments 54-64, wherein one or more cerebrospinal fluid samples are utilized for reduction assessment.

66. The method of any one of Embodiments 54-65, wherein wild type HTT transcript level is not significantly reduced.

67. The method of any one of Embodiments 54-66, wherein wild type HTT protein level is not significantly reduced.

68. The method of any one of Embodiments 54-67, wherein total HTT transcript level is not significantly reduced.

69. The method of any one of Embodiments 54-68, wherein total HTT protein level is not significantly reduced.

70. The method of any one of Embodiments 54-69, wherein neurofilament light chain (NfL) level in CSF is not significantly increased.

71. The method of any one of Embodiments 54-70, wherein assessment is performed after about 1, 2, 3, 4, 5, 6, 7, 8, 9, 10, 11, 12, 13, 14, 15, 16, 17, 18, 19, or 20 days, or about 3, 4, 5, 6, 7, or 8 weeks, or about 3, 4, 5, or 6 or more months after administration of a dose and before a next dose, if any, is administered.

72. The method of any one of Embodiments 54-71, wherein assessment is performed after about 1, 2, 3, 4, 5, 6, 7, 8, 9, 10, 11, 12, 13, 14, 15, 16, 17, 18, 19, or 20 doses are administered.

73. The method of any one of Embodiments 54-72, wherein onset of and/or severity of a symptom of Huntington's disease in a subject is delayed and/or reduced.

74. The method of any one of the previous Embodiments, wherein the subject is administered a steroid at least about one month prior to the first dose of WVE-003.

75. The method of any one of the previous Embodiments, wherein the subject is administered two or more doses of WVE-003, and the interval between any two of the neighboring doses is about a month or about 4 weeks.

76. The method of any one of the previous Embodiments, wherein the subject is administered two or more doses of WVE-003, and the interval between any two of the neighboring doses is about two months or about 8 weeks.

77. The method of any one of the previous Embodiments, wherein the subject is administered two or more doses of WVE-003, and the interval between any two of the neighboring doses is about three months or about 12 weeks.

78. The method of any one of the previous Embodiments, wherein the subject is administered two or more doses of WVE-003, approximately monthly or approximately once every 4 weeks.

79. The method of any one of the previous Embodiments, wherein the subject is administered two or more doses of WVE-003, approximately once every 2 months or approximately once every 8 weeks.

80. The method of any one of the previous Embodiments, wherein the subject is administered two or more doses of WVE-003, approximately once every 3 months or approximately once every 12 weeks.

81. The method of any one of the previous Embodiments, wherein the subject is administered WVE-003 approximately monthly for at least about 2 months.

82. The method of any one of the previous Embodiments, wherein the subject is administered WVE-003 approximately monthly for at least about 4 months.

83. The method of any one of the previous Embodiments, wherein the subject is administered WVE-003 approximately monthly for at least about 8 months.

84. The method of any one of the previous Embodiments, wherein the subject is administered WVE-003 approximately once every 4 weeks for at least about 8 weeks.

85. The method of any one of the previous Embodiments, wherein the subject is administered WVE-003 approximately once every 4 weeks for at least about 12 weeks.

86. The method of any one of the previous Embodiments, wherein the subject is administered WVE-003 approximately once every 4 weeks for at least about 16 weeks.

87. The method of any one of the previous Embodiments, wherein the subject is administered WVE-003 approximately once every 8 weeks for at least about 8 weeks.

88. The method of any one of the previous Embodiments, wherein the subject is administered WVE-003 approximately once every 8 weeks for at least about 16 weeks.

89. The method of any one of the previous Embodiments, wherein the subject is administered WVE-003 approximately once every 12 weeks for at least about 12 weeks.

90. The method of any one of the previous Embodiments, wherein the subject is administered WVE-003 approximately once every 12 weeks for at least about 24 weeks.

91. The method of any one of the previous Embodiments, wherein the subject is administered WVE-003 approximately monthly for at least about 12 months.

92. The method of any one of the previous Embodiments, wherein the subject is administered WVE-003 approximately monthly for at least about 16 months.

93. The method of any one of the previous Embodiments, wherein the subject is administered WVE-003 approximately monthly for at least about 48 months.

94. The method of any one of the previous Embodiments, wherein WVE-003 is delivered intrathecally.

95. The method of any one of the previous Embodiments, wherein the method further comprises the step of confirming that the subject has a mutation in the HTT gene that is amenable to an allele-specific knockdown of the mutant HTT gene or a gene product thereof transcript.

96. The method of any one of the previous Embodiments, wherein WVE-003 is administered in a salt form.

97. The method of any one of the previous Embodiments, wherein WVE-003 is administered in a sodium salt form.

98. The method of any one of the previous Embodiments, wherein WVE-003 is formulated as a liquid formulation.

99. The method of any one of the previous Embodiments, wherein WVE-003 is formulated as a liquid formulation, wherein the liquid formulation comprises WVE-003, sodium chloride, and water.

100. The method of any one of the previous Embodiments, wherein WVE-003 is formulated as a liquid formulation, wherein the liquid formulation is reconstituted from a lyophilized preparation.

101. The method of any one of the previous Embodiments, wherein the level, expression and/or activity of a mHTT transcript or gene product thereof is reduced by at least about 5%.

102. The method of any one of the previous Embodiments, wherein the level, expression and/or activity of a mHTT transcript or gene product thereof is reduced by at least about 10%.

103. A composition comprising WVE-003 or a composition thereof, wherein WVE-003 or the composition thereof is in a solid form.

104. A composition comprising WVE-003 or a composition thereof, wherein WVE-003 or the composition thereof is lyophilized.

105. A composition comprising WVE-003 or a composition thereof, wherein WVE-003 or the composition thereof is present in a vial in an amount of about 20 mg.

106. A composition comprising WVE-003 or a composition thereof, wherein WVE-003 or the composition thereof is present in a vial in an amount of about 20 mg, and the vial is backfilled with nitrogen.

107. A composition comprising WVE-003 or a composition thereof, wherein WVE-003 or the composition thereof is diluted with a solution of sodium chloride.

108. A composition comprising WVE-003 or a composition thereof, wherein WVE-003 or the composition thereof is diluted with 0.9% sodium chloride.

109. A composition comprising WVE-003 or a composition thereof, wherein the composition consists essentially of WVE-003 or a composition thereof, sodium chloride, and water.

110. A composition comprising WVE-003 or a composition thereof, wherein the composition consists essentially of WVE-003 or a composition thereof and aCSF.

111. The composition of any one of Embodiments 103-110, wherein WVE-003 is in a pharmaceutically acceptable salt form.

112. The method or composition of any one of the preceding Embodiments, wherein one or more pharmaceutically acceptable salt forms of WVE-003 is administered or in the composition.

113. The method or composition of any one of the preceding Embodiments, wherein the amount of WVE-003 includes the amount of one or more pharmaceutically acceptable salt forms, each of which is independently converted to the amount of the acid form.

114. A method for treating Huntington's disease, comprising administering or delivering to a subject suffering therefrom WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001Rm5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

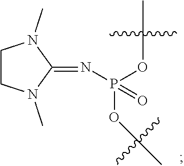 [SMILES: *OP(=O)(N=C1N(C)CCN1C)O*]

eo represents a 2′-OCH2CH2OCH3 modification to a nucleoside; and
*R represents a Rp phosphorothioate linkage.

115. A method for preventing Huntington's disease, comprising administering or delivering to a subject suffering therefrom WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001Rm5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

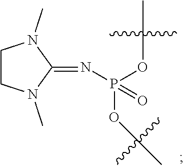 [SMILES: *OP(=O)(N=C1N(C)CCN1C)O*]

eo represents a 2′-OCH2CH2OCH3 modification to a nucleoside; and
*R represents a Rp phosphorothioate linkage.

116. A method, comprising administering or delivering to a subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001Rm5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

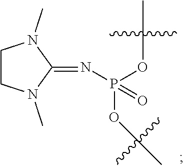 [SMILES: *OP(=O)(N=C1N(C)CCN1C)O*]

eo represents a 2′-OCH2CH2OCH3 modification to a nucleoside; and
*R represents a Rp phosphorothioate linkage.

117. A method for decreasing the activity, expression, and/or level of a mutant HTT gene or its gene product in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001Rm5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

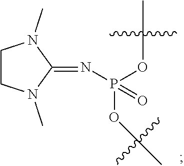 [SMILES: *OP(=O)(N=C1N(C)CCN1C)O*]

eo represents a 2′-OCH2CH2OCH3 modification to a nucleoside; and
*R represents a Rp phosphorothioate linkage.

118. A method for preferential knockdown of a repeat expansion-containing HTT RNA transcript relative to a non-repeat expansion-containing HTT RNA transcript in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001Rm5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

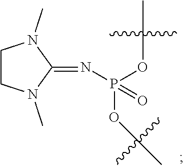 [SMILES: *OP(=O)(N=C1N(C)CCN1C)O*]

eo represents a 2′-OCH2CH2OCH3 modification to a nucleoside; and
*R represents a Rp phosphorothioate linkage.

119. A method for reducing level of a HTT transcript comprising CAG repeat expansion in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001R m5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of;

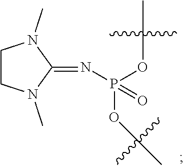 [SMILES: *OP(=O)(N=C1N(C)CCN1C)O*]

eo represents a 2′-OCH2CH2OCH3 modification to a nucleoside; and
*R represents a Rp phosphorothioate linkage.

120. A method for reducing level of a product of a HTT transcript comprising CAG repeat expansion in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form, wherein WVE-003 is:
mG*S mUn001RmU mGn001RmA*ST*SC*ST*SG*ST*RA*SG*SC*SA*SG*R m5Ceon001RAeoGeon001Rm5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of

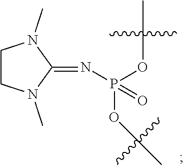 [SMILES: *OP(=O)(N=C1N(C)CCN1C)O*]

eo represents a 2′-OCH2CH2OCH3 modification to a nucleoside; and
*R represents a Rp phosphorothioate linkage.

121. The method of Embodiment 120, wherein the product is a polypeptide.

122. The method of Embodiment 120, wherein the product is a polypeptide comprising expanded poly-Q.

123. The method of any one of the preceding Embodiments, wherein two or more doses, e.g., 2, 3, 4, 5, 6, 7, 8, 9, 10, 11, 12, 13, 14, 15, 16, 17, 18, 19, 20 or more doses are administered.

124. The method of any one of the preceding Embodiments, wherein a dose of WVE-003 is administered in one or more forms.

125. The method of any one of the preceding Embodiments, wherein a dose of WVE-003 is administered in one or more pharmaceutically acceptable salt forms.

126. The method of any one of Embodiments 124-125, wherein one form is WVE-003 pentadecasodium salt.

127. The method of any one of Embodiments 124-126, wherein a dose of WVE-003 is administered in a pharmaceutical composition comprising WVE-003 and a pharmaceutically acceptable carrier.

128. The method of any one of Embodiments 124-126, wherein a dose of WVE-003 is administered in a pharmaceutical composition comprising a WVE-003 pentadecasodium salt and a pharmaceutically acceptable carrier.

129. The method of any one of Embodiments 124-126, wherein a dose of WVE-003 is administered in a pharmaceutical composition consisting of WVE-003 pentadecasodium salt and a pharmaceutically acceptable carrier.

130. The method of any one of Embodiments 124-126, wherein a dose of WVE-003 is administered in a pharmaceutical composition consisting of WVE-003 pentadecasodium salt dissolved in a pharmaceutically acceptable carrier.

131. The method of any one of the preceding Embodiments, wherein each dose of WVE-003 is independently administered in one or more pharmaceutically acceptable salt forms.

132. The method of Embodiment 131, wherein one form is WVE-003 pentadecasodium salt.

133. The method of any one of the preceding Embodiments, wherein each dose of WVE-003 is independently administered in a pharmaceutical composition comprising WVE-003 and a pharmaceutically acceptable carrier.

134. The method of any one of the preceding Embodiments, wherein each dose of WVE-003 is independently administered in a pharmaceutical composition comprising a WVE-003 pentadecasodium salt and a pharmaceutically acceptable carrier.

135. The method of any one of the preceding Embodiments, wherein each dose of WVE-003 is independently administered in a pharmaceutical composition consisting of WVE-003 pentadecasodium salt and a pharmaceutically acceptable carrier.

136. The method of any one of the preceding Embodiments, wherein each dose of WVE-003 is independently administered in a pharmaceutical composition consisting of WVE-003 pentadecasodium salt dissolved in a pharmaceutically acceptable carrier.

137. A method for treating Huntington's disease, comprising administering or delivering to a subject suffering therefrom WVE-003 pentadecasodium salt:

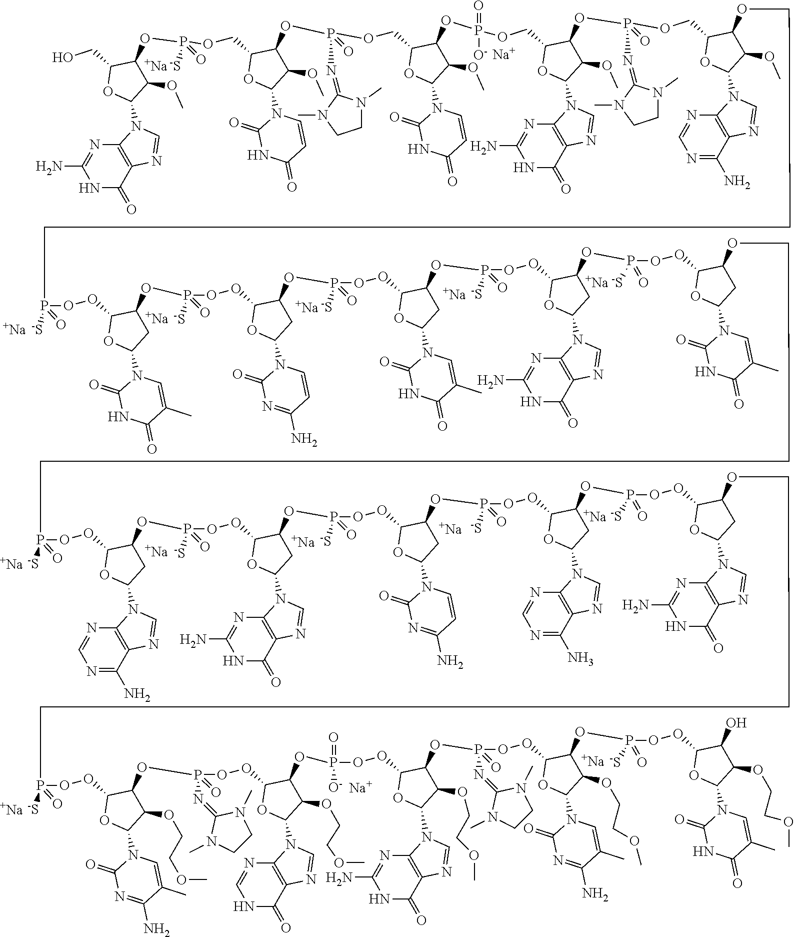

at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

138. A method for preventing Huntington's disease, comprising administering or delivering to a subject suffering therefrom WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

139. A method, comprising administering or delivering to a subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

140. A method for decreasing the activity, expression, and/or level of a mutant HTT gene or its gene product in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

141. A method for preferential knockdown of a repeat expansion-containing HTT RNA transcript relative to a non-repeat expansion-containing HTT RNA transcript in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid forM.

142. A method for reducing level of a HTT transcript comprising CAG repeat expansion in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

143. A method for reducing level of a product of a HTT transcript comprising CAG repeat expansion in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

144. The method of Embodiment 143, wherein the product is a polypeptide.

145. The method of Embodiment 143, wherein the product is a polypeptide comprising expanded poly-Q.

146. The method of any one of Embodiments 137-145, wherein two or more doses, e.g., 2, 3, 4, 5, 6, 7, 8, 9, 10, 11, 12, 13, 14, 15, 16, 17, 18, 19, 20 or more doses are administered.

147. The method of any one of Embodiments 137-146, wherein a dose of WVE-003 pentadecasodium salt is administered in a pharmaceutical composition comprising WVE-003 pentadecasodium salt and a pharmaceutically acceptable carrier.

148. The method of any one of Embodiments 137-146, wherein a dose of WVE-003 pentadecasodium salt is administered in a pharmaceutical composition consisting of WVE-003 pentadecasodium salt and a pharmaceutically acceptable carrier.

149. The method of any one of Embodiments 137-146, wherein a dose of WVE-003 pentadecasodium salt is administered in a pharmaceutical composition consisting of WVE-003 pentadecasodium salt dissolved in a pharmaceutically acceptable carrier.

150. The method of any one of the preceding Embodiments, wherein each dose of WVE-003 pentadecasodium salt is independently administered in a pharmaceutical composition comprising WVE-003 and a pharmaceutically acceptable carrier.

151. The method of any one of the preceding Embodiments, wherein each dose of WVE-003 pentadecasodium salt is independently administered in a pharmaceutical composition consisting of WVE-003 pentadecasodium salt and a pharmaceutically acceptable carrier.

152. The method of any one of the preceding Embodiments, wherein each dose of WVE-003 pentadecasodium salt is independently administered in a pharmaceutical composition consisting of WVE-003 pentadecasodium salt dissolved in a pharmaceutically acceptable carrier.

153. The method of any one of the preceding Embodiments, wherein the pharmaceutically acceptable carrier is aCSF.

154. The method of any one of the preceding Embodiments, wherein a dose is equivalent to about 10 mg WVE-003 free acid form.

155. The method of any one of the preceding Embodiments, wherein a dose is equivalent to about 20 mg WVE-003 free acid form.

156. The method of any one of the preceding Embodiments, wherein a dose is equivalent to about 30 mg WVE-003 free acid form.

157. The method of any one of the preceding Embodiments, wherein a dose is equivalent to about 40 mg WVE-003 free acid form.

158. The method of any one of the preceding Embodiments, wherein a dose is equivalent to about 50 mg WVE-003 free acid form.

159. The method of any one of the preceding Embodiments, wherein a dose is equivalent to about 60 mg WVE-003 free acid form.

160. The method of any one of the preceding Embodiments, wherein a dose is equivalent to about 70 mg WVE-003 free acid form.

161. The method of any one of the preceding Embodiments, wherein a dose is equivalent to about 80 mg WVE-003 free acid form.

162. The method of any one of the preceding Embodiments, wherein a dose is equivalent to about 90 mg WVE-003 free acid form.

163. The method of any one of the preceding Embodiments, wherein a dose is equivalent to about 100 mg WVE-003 free acid form.

164. The method of any one of the preceding Embodiments, wherein a dose is equivalent to about 110 mg WVE-003 free acid form.

165. The method of any one of the preceding Embodiments, wherein a dose is equivalent to about 120 mg WVE-003 free acid form.

166. The method of any one of the preceding Embodiments, wherein a dose is equivalent to about 130 mg WVE-003 free acid form.

167. The method of any one of the preceding Embodiments, wherein a dose is equivalent to about 140 mg WVE-003 free acid form.

168. The method of any one of the preceding Embodiments, wherein a dose is equivalent to about 150 mg WVE-003 free acid form.

169. The method of any one of the preceding Embodiments, wherein a dose is equivalent to about 160 mg WVE-003 free acid form.

170. The method of any one of the preceding Embodiments, wherein a dose is equivalent to about 170 mg WVE-003 free acid form.

171. The method of any one of Embodiments 114-153, wherein each dose is independently equivalent to about 10 mg WVE-003 free acid form.

172. The method of any one of Embodiments 114-153, wherein each dose is independently equivalent to about 20 mg WVE-003 free acid form.

173. The method of any one of Embodiments 114-153, wherein each dose is independently equivalent to about 30 mg WVE-003 free acid form.

174. The method of any one of Embodiments 114-153, wherein each dose is independently equivalent to about 40 mg WVE-003 free acid form.

175. The method of any one of Embodiments 114-153, wherein each dose is independently equivalent to about 50 mg WVE-003 free acid form.

176. The method of any one of Embodiments 114-153, wherein each dose is independently equivalent to about 60 mg WVE-003 free acid form.

177. The method of any one of Embodiments 114-153, wherein each dose is independently equivalent to about 70 mg WVE-003 free acid form.

178. The method of any one of Embodiments 114-153, wherein each dose is independently equivalent to about 80 mg WVE-003 free acid form.

179. The method of any one of Embodiments 114-153, wherein each dose is independently equivalent to about 90 mg WVE-003 free acid form.

180. The method of any one of Embodiments 114-153, wherein each dose is independently equivalent to about 100 mg WVE-003 free acid form.

181. The method of any one of Embodiments 114-153, wherein each dose is independently equivalent to about 110 mg WVE-003 free acid form.

182. The method of any one of Embodiments 114-153, wherein each dose is independently equivalent to about 120 mg WVE-003 free acid form.

183. The method of any one of Embodiments 114-153, wherein each dose is independently equivalent to about 130 mg WVE-003 free acid form.

184. The method of any one of Embodiments 114-153, wherein each dose is independently equivalent to about 140 mg WVE-003 free acid form.

185. The method of any one of Embodiments 114-153, wherein each dose is independently equivalent to about 150 mg WVE-003 free acid form.

186. The method of any one of Embodiments 114-153, wherein each dose is independently equivalent to about 160 mg WVE-003 free acid form.

187. The method of any one of Embodiments 114-153, wherein each dose is independently equivalent to about 170 mg WVE-003 free acid form.

188. The method of any one of the preceding Embodiments, wherein two or more consecutive doses are administered independently about every 1, 2, 3, 4, 5, 6, 7, 8, 9, 10, 11, 12 weeks, or about every 3, 4, 5, 6, 7, 8, 9, 10, 11, 12 or more months.

189. The method of any one of the preceding Embodiments, wherein 10 or more consecutive doses are administered independently about every 1, 2, 3, 4, 5, 6, 7, 8, 9, 10, 11, 12 weeks, or about every 3, 4, 5, 6, 7, 8, 9, 10, 11, 12 or more months.

190. The method of any one of the preceding Embodiments, wherein all doses are administered independently about every 1, 2, 3, 4, 5, 6, 7, 8, 9, 10, 11, 12 weeks, or about every 3, 4, 5, 6, 7, 8, 9, 10, 11, 12 or more months.

191. The method of any one of the preceding Embodiments, wherein two or more consecutive doses are administered about every two weeks.

192. The method of any one of the preceding Embodiments, wherein two or more consecutive doses are administered about every four weeks.

193. The method of any one of the preceding Embodiments, wherein two or more consecutive doses are administered about every six weeks.

194. The method of any one of the preceding Embodiments, wherein two or more consecutive doses are administered about every eight weeks.

195. The method of any one of the preceding Embodiments, wherein two or more consecutive doses, each independently equivalent to about 30 mg WVE-003 free acid form, are administered about every eight weeks.

196. The method of any one of the preceding Embodiments, wherein two or more consecutive doses are administered about every twelve weeks.

197. The method of any one of the preceding Embodiments, wherein two or more consecutive doses, each independently equivalent to about 30 mg WVE-003 free acid form, are administered about every twelve weeks.

198. The method of any one of the preceding Embodiments, wherein two or more consecutive doses are administered about monthly.

199. The method of any one of the preceding Embodiments, wherein two or more consecutive doses are administered about every two months.

200. The method of any one of the preceding Embodiments, wherein two or more consecutive doses are administered about every two months or less frequently.

201. The method of any one of the preceding Embodiments, wherein two or more consecutive doses are administered about every three months.

202. The method of any one of the preceding Embodiments, wherein two or more consecutive doses are administered about every four months.

203. The method of any one of the preceding Embodiments, wherein two or more consecutive doses are administered about quarterly or less frequently.

204. The method of any one of the preceding Embodiments, wherein two or more consecutive doses are administered about quarterly.

205. The method of any one of Embodiments 114-189, wherein all doses are administered about every 8 weeks or less frequently.

206. The method of any one of Embodiments 114-189, wherein all doses are administered about every 8 weeks.

207. The method of any one of Embodiments 114-189, wherein all doses are each independently equivalent to about 30 mg WVE-003 free acid form and are administered about every 8 weeks.

208. The method of any one of Embodiments 114-189, wherein all doses are administered about every 12 weeks or less frequently.

209. The method of any one of Embodiments 114-189, wherein all doses are administered about every 12 weeks.

210. The method of any one of Embodiments 114-189, wherein all doses are each independently equivalent to about 30 mg WVE-003 free acid form and are administered about every 12 weeks.

211. The method of any one of Embodiments 114-189, wherein all doses are administered about every two months or less frequently.

212. The method of any one of Embodiments 114-189, wherein all doses are administered about every two months.

213. The method of any one of Embodiments 114-189, wherein all doses are administered about quarterly or less frequently.

214. The method of any one of Embodiments 114-189, wherein all doses are administered about quarterly.

215. The method of any one of Embodiments 114-189, wherein all doses are administered about monthly.

216. The method of any one of the preceding Embodiments, wherein the subject has expanded CAG repeat region in a HTT gene.

217. The method of any one of the preceding Embodiments, wherein the subject expresses a HTT transcript comprising an expanded CAG repeat region.

218. The method of any one of the preceding Embodiments, wherein the A variant of rs362273 is on the same allele as the expanded CAG repeat region in a HTT gene.

219. The method of any one of the preceding Embodiments, wherein the expanded CAG repeat region comprises 36 or more CAG repeats.

220. The method of any one of the preceding Embodiments, wherein the expanded CAG repeat region comprises 40 or more CAG repeats.

221. The method of any one of the preceding Embodiments, wherein level of mHTT transcript in cerebrospinal fluid is reduced by about 10%, 20%, 30%, 40%, 50% or more about or after about 1, 2, 3, 4, 5, 6, 7, 8, 9, 10, 11, 12, 13, 14, 15, 16, 17, 18, 19, 20 or more weeks after a first dose.

222. The method of any one of the preceding Embodiments, wherein level of mHTT polypeptide in cerebrospinal fluid is reduced by about 10%, 20%, 30%, 40%, 50% or more about or after about 1, 2, 3, 4, 5, 6, 7, 8, 9, 10, 11, 12, 13, 14, 15, 16, 17, 18, 19, 20 or more weeks after a first dose.

223. The method of any one of the preceding Embodiments, wherein the level is reduced by about 10% or more.

224. The method of any one of the preceding Embodiments, wherein the level is reduced by about 20% or more.

225. The method of any one of the preceding Embodiments, wherein the level is reduced by about 30% or more.

226. The method of any one of the preceding Embodiments, wherein the level is reduced by about 40% or more.

227. The method of any one of the preceding Embodiments, wherein the level is reduced by about 50% or more.

228. The method of any one of the preceding Embodiments, wherein level of wtHTT transcript or polypeptide is reduced by no more than about 10%, 20%, 30%, 40% or 50%.

229. The method of any one of the preceding Embodiments, wherein level of wtHTT transcript or polypeptide is reduced by no more than about 10%.

230. The method of any one of the preceding Embodiments, wherein level of wtHTT transcript or polypeptide is reduced by no more than about 20%.

231. The method of any one of the preceding Embodiments, wherein level of wtHTT transcript or polypeptide is reduced by no more than about 30%.

232. The method of any one of the preceding Embodiments, wherein level of wtHTT transcript or polypeptide is reduced by no more than about 40%.

233. The method of any one of the preceding Embodiments, wherein level of wtHTT transcript or polypeptide is reduced by no more than about 50%.

234. The method of any one of the preceding Embodiments, wherein about is ±1%.

235. The method of any one of the preceding Embodiments, wherein about is ±2%,

236. The method of any one of the preceding Embodiments, wherein about is ±3%.

237. The method of any one of the preceding Embodiments, wherein about is ±4%.

238. The method of any one of the preceding Embodiments, wherein about is ±5%.

239. The method of any one of the preceding Embodiments, wherein about is ±6%.

240. The method of any one of the preceding Embodiments, wherein about is ±7%.

241. The method of any one of the preceding Embodiments, wherein about is ±8%.

242. The method of any one of the preceding Embodiments, wherein about is ±9%.

243. The method of any one of the preceding Embodiments, wherein about is ±10%.

244. The method of any one of the preceding Embodiments, wherein a pharmaceutical composition has a pH of about 6-8.

245. The method of any one of the preceding Embodiments, wherein a pharmaceutical composition has a pH of about 6.4-7.2.

246. The method of Embodiment 244Embodiment, wherein a pharmaceutical composition has a pH of about 7.3.

247. The method of Embodiment 244, wherein a pharmaceutical composition has a pH of about 7.4.

248. The method of any one of the preceding Embodiments, wherein the composition has a purity of about 80%-90%.

249. The method of any one of the preceding Embodiments, wherein the composition has a purity of about 82% or more.

250. The method of any one of the preceding Embodiments, wherein the composition has a purity of about 84% or more.

251. The method of any one of the preceding Embodiments, wherein the composition has a purity of about 85% or more.

252. The method of any one of the preceding Embodiments, wherein the composition has a purity of about 86% or more.

253. The method of any one of the preceding Embodiments, wherein the composition has a purity of about 87% or more.

254. The method of any one of the preceding Embodiments, wherein the composition has a purity of about 88% or more.

255. The method of any one of the preceding Embodiments, wherein the composition has a purity of about 89% or more.

256. The method of any one of the preceding Embodiments, wherein the composition has a purity of about 90% or more.

257. The method of any one of Embodiments 248-256, wherein the purity is measured by IP-RP-UPLC using area % at 260 nm.

258. The method of any one of Embodiments 248-257, wherein the purity is measured by IP-RP-UPLC using area % at 260 nm and the Set A parameters.

259. The method of any one of Embodiments 248-258, wherein the purity is measured by an IP-RP-UPLC method for purity as described herein.

260. The method of any one of the preceding Embodiments, wherein impurities in the composition are no more than about 10%-20%.

261. The method of any one of the preceding Embodiments, wherein impurities in the composition are no more than about or about 20%.

262. The method of any one of the preceding Embodiments, wherein impurities in the composition are no more than about or about 19%.

263. The method of any one of the preceding Embodiments, wherein impurities in the composition are no more than about or about 18%.

264. The method of any one of the preceding Embodiments, wherein impurities in the composition are no more than about or about 17%.

265. The method of any one of the preceding Embodiments, wherein impurities in the composition are no more than about or about 16%.

266. The method of any one of the preceding Embodiments, wherein impurities in the composition are no more than about or about 15%.

267. The method of any one of the preceding Embodiments, wherein impurities in the composition are no more than about or about 14%.

268. The method of any one of the preceding Embodiments, wherein impurities in the composition are no more than about or about 13%.

269. The method of any one of the preceding Embodiments, wherein impurities in the composition are no more than about or about 12%.

270. The method of any one of the preceding Embodiments, wherein impurities in the composition are no more than about or about 110%.

271. The method of any one of the preceding Embodiments, wherein impurities in the composition are no more than about or about 10%.

272. The method of any one of Embodiments 260-271, wherein the impurities are measured by IP-RP-UPLC using area % at 260 nm.

273. The method of any one of Embodiments 260-272, wherein the impurities are measured by IP-RP-UPLC using area % at 260 nm and the Set A parameters.

274. The method of any one of Embodiments 260-273, wherein the impurities are measured by an IP-RP-UPLC method for purity as described herein.

275. The method of any one of the preceding Embodiments, wherein stereochemical purity of WVE-003 is about 80% or more.

276. The method of any one of the preceding Embodiments, wherein stereochemical purity of WVE-003 is about 83% or more.

277. The method of any one of the preceding Embodiments, wherein stereochemical purity of WVE-003 is about 80%-85%.

278. The method of any one of the preceding Embodiments, wherein stereochemical purity is assessed by dimer modeling.

279. The method of any one of the preceding Embodiments, wherein the amount of WVE-003 is measured by UV at 260 nm and 25.0 OD/mg.

280. The method of any one of the preceding Embodiments, wherein a WVE-003 drug substance is manufactured by a process described herein.

281. The method of any one of the preceding Embodiments, wherein a WVE-003 drug substance is characterized by one or more methods described herein.

282. The method of any one of the preceding Embodiments, wherein stereochemical identity of WVE-003 is confirmed by IP-RP-UPLC.

283. The method of any one of the preceding Embodiments, wherein stereochemical identity of WVE-003 is confirmed by IP-RP-UPLC according to Set B parameters.

284. The method of any one of the preceding Embodiments, wherein stereochemical identity of WVE-003 is confirmed by an IP-RP-UPLC method for stereochemical identity as described herein.

285. The method of any one of the preceding Embodiments, wherein a WVE-003 drug substance is released by one or more methods described herein.

286. The method of any one of the preceding Embodiments, wherein a WVE-003 drug substance is stored by one or more methods described herein.

287. The method of any one of Embodiments 280-286, wherein the WVE-003 drug substance is pentadecasodium salt.

288. The method of any one of the preceding Embodiments, wherein a WVE-003 drug product is manufactured by a process described herein.

289. The method of any one of the preceding Embodiments, wherein a WVE-003 drug product is characterized by one or more methods described herein.

290. The method of any one of the preceding Embodiments, wherein a WVE-003 drug product is released by one or more methods described herein.

291. The method of any one of the preceding Embodiments, wherein a WVE-003 drug product is stored by one or more methods described herein.

292. The method of any one of the preceding Embodiments, wherein a pharmaceutical composition is manufactured by a process described herein.

293. The method of any one of the preceding Embodiments, wherein a pharmaceutical composition is characterized by one or more methods described herein.

294. The method of any one of the preceding Embodiments, wherein a pharmaceutical composition is released by one or more methods described herein.

295. The method of any one of the preceding Embodiments, wherein a pharmaceutical composition is stored by one or more methods described herein.

296. The method of any one of the preceding Embodiments, wherein WVE-003 is administered intrathecally.

297. The method of any one of the preceding Embodiments, wherein WVE-003 is administered by direct lumber injection.

298. The method of any one of the preceding Embodiments, wherein the method provides reduction of level of the repeat expansion-containing HTT transcript as measured by percentage that is at least 1.1, 1.2, 1.3, 1.4, 1.5, 1.6, 1.7, 1.8, 1.9, 2, 2.5, 3, 4, 5, 6, 7, 8, 9, or 10 fold of the reduction of level of the non-repeat-expansion-containing HTT transcript as measured by percentage.

299. The method of any one of the preceding Embodiments, wherein the subject improves in functional assessments.

300. The method of any one of the preceding Embodiments, wherein an improvement is compared to baseline.

301. The method of any one of the preceding Embodiments, wherein an improvement is compared to absence of WVE-003 administration.

302. The method of any one of the preceding Embodiments, wherein an improvement is compared to administration of a reference composition.

303. The method of any one of the preceding Embodiments, wherein a reference composition is comparable to an administered WVE-003 composition but does not contain WVE-003.

304. The method of any one of the preceding Embodiments, wherein a dose is administered as WVE-003 pentadecasodium salt dissolved in aCSF.

305. The method of any one of the preceding Embodiments, wherein a dose is administered in a 20 mL aCSF solution.

306. The method of any one of the preceding Embodiments, wherein 20 mL CSF is taken out from a subject before administration of a dose.

307. The method of any one of the preceding Embodiments, wherein the subject is about 25 years old or older.

308. The method of any one of the preceding Embodiments, wherein the subject is about 60 years old or younger.

309. The method of any one of the preceding Embodiments, wherein the subject is with early manifest Huntington's disease.

310. The method of any one of the previous Embodiments, wherein the subject receives or is exposed to an additional therapeutic agent.

311. A composition comprising WVE-003.

312. The composition of Embodiment 311, wherein a form of WVE-003 in the composition is a pharmaceutically acceptable salt form.

313. The composition of any one of Embodiments 311-312, wherein each form of WVE-003 in the composition is independently a salt form.

314. The composition of any one of Embodiments 311-313, wherein each form of WVE-003 in the composition is independently a pharmaceutically acceptable salt form.

315. The composition of any one of Embodiments 311-314, wherein a form of WVE-003 in the composition is WVE-003 pentadecasodium salt.

316. The composition of any one of Embodiments 311-314, wherein WVE-003 exists as WVE-003 pentadecasodium salt in the composition.

317. The composition of any one of the Embodiments 311-316, wherein the composition is a drug substance.

318. The composition of any one of Embodiments 311-316, wherein the composition is a drug product.

319. The compound of any one of the preceding Embodiments, wherein the composition is a liquid composition wherein WVE-003 is dissolved.

320. The composition of any one of the preceding Embodiments, wherein the composition is a pharmaceutical composition further comprising a pharmaceutically acceptable carrier.

321. The composition of Embodiment 320, wherein a pharmaceutically acceptable carrier is or comprises artificial cerebrospinal fluid (aCSF).

322. The composition of any one of the preceding Embodiments, wherein the composition is isotonic.

323. The composition of any one of the preceding Embodiments, wherein the composition has a pH of about 6-8.

324. The composition of any one of the preceding Embodiments, wherein the composition has a pH of about 6.4-7.2.

325. The composition of Embodiment 323Embodiment, wherein the composition has a pH of about 7.3.

326. The composition of Embodiment 323, wherein a pharmaceutical composition has a pH of about 7.4.

327. The composition of any one of Embodiments 311-318, wherein the composition is lyophilized WVE-003 powder.

328. The composition of any one of the preceding Embodiments, wherein WVE-003 in the composition is equivalent to about 5 mg WVE-003 free acid form.

329. The composition of any one of the preceding Embodiments, wherein WVE-003 in the composition is equivalent to about 10 mg WVE-003 free acid form.

330. The composition of any one of the preceding Embodiments, wherein WVE-003 in the composition is equivalent to about 20 mg WVE-003 free acid form.

331. The composition of any one of the preceding Embodiments, wherein WVE-003 in the composition is equivalent to about 30 mg WVE-003 free acid form.

332. The composition of any one of the preceding Embodiments, wherein WVE-003 in the composition is equivalent to about 40 mg WVE-003 free acid form.

333. The composition of any one of the preceding Embodiments, wherein WVE-003 in the composition is equivalent to about 50 mg WVE-003 free acid form.

334. The composition of any one of the preceding Embodiments, wherein WVE-003 in the composition is equivalent to about 60 mg WVE-003 free acid form.

335. The composition of any one of the preceding Embodiments, wherein WVE-003 in the composition is equivalent to about 70 mg WVE-003 free acid form.

336. The composition of any one of the preceding Embodiments, wherein WVE-003 in the composition is equivalent to about 80 mg WVE-003 free acid form.

337. The composition of any one of the preceding Embodiments, wherein WVE-003 in the composition is equivalent to about 90 mg WVE-003 free acid form.

338. The composition of any one of the preceding Embodiments, wherein WVE-003 in the composition is equivalent to about 100 mg WVE-003 free acid form.

339. The composition of any one of the preceding Embodiments, wherein WVE-003 in the composition is equivalent to about 110 mg WVE-003 free acid form.

340. The composition of any one of the preceding Embodiments, wherein WVE-003 in the composition is equivalent to about 120 mg WVE-003 free acid form.

341. The composition of any one of the preceding Embodiments, wherein WVE-003 in the composition is equivalent to about 130 mg WVE-003 free acid form.

342. The composition of any one of the preceding Embodiments, wherein WVE-003 in the composition is equivalent to about 140 mg WVE-003 free acid form.

343. The composition of any one of the preceding Embodiments, wherein WVE-003 in the composition is equivalent to about 150 mg WVE-003 free acid form.

344. The composition of any one of the preceding Embodiments, wherein WVE-003 in the composition is equivalent to about 160 mg WVE-003 free acid form.

345. The composition of any one of the preceding Embodiments, wherein WVE-003 in the composition is equivalent to about 170 mg WVE-003 free acid form.

346. The composition of any one of the preceding Embodiments, wherein the composition is packaged into a vial.

347. The composition of any one of the preceding Embodiments, wherein the composition is reconstituted and diluted in artificial cerebrospinal fluid (aCSF).

348. The composition of any one of the preceding Embodiments, wherein the composition has a purity of about 70%-90%.

349. The composition of any one of the preceding Embodiments, wherein the composition has a purity of about 80%-90%.

350. The composition of any one of the preceding Embodiments, wherein the composition has a purity of about 80% or more.

351. The composition of any one of the preceding Embodiments, wherein the composition has a purity of about 81% or more.

352. The composition of any one of the preceding Embodiments, wherein the composition has a purity of about 82% or more.

353. The composition of any one of the preceding Embodiments, wherein the composition has a purity of about 83% or more.

354. The composition of any one of the preceding Embodiments, wherein the composition has a purity of about 84% or more.

355. The composition of any one of the preceding Embodiments, wherein the composition has a purity of about 85% or more.

356. The composition of any one of the preceding Embodiments, wherein the composition has a purity of about 86% or more.

357. The composition of any one of the preceding Embodiments, wherein the composition has a purity of about 87% or more.

358. The composition of any one of the preceding Embodiments, wherein the composition has a purity of about 88% or more.

359. The composition of any one of the preceding Embodiments, wherein the composition has a purity of about 89% or more.

360. The composition of any one of the preceding Embodiments, wherein the composition has a purity of about 90% or more.

361. The composition of any one of Embodiments 347-360, wherein the purity is measured by IP-RP-UPLC using area % at 260 nm.

362. The composition of any one of Embodiments 347-361, wherein the purity is measured by IP-RP-UPLC using area % at 260 nm and the Set A parameters.

363. The composition of any one of Embodiments 347-362, wherein the purity is measured by an IP-RP-UPLC method for purity as described herein.

364. The composition of any one of the preceding Embodiments, wherein impurities in the composition are no more than about 10%-30%.

365. The composition of any one of the preceding Embodiments, wherein impurities in the composition are no more than about 10%-20%.

366. The composition of any one of the preceding Embodiments, wherein impurities in the composition are no more than about or about 20%.

367. The composition of any one of the preceding Embodiments, wherein impurities in the composition are no more than about or about 19%.

368. The composition of any one of the preceding Embodiments, wherein impurities in the composition are no more than about or about 18%.

369. The composition of any one of the preceding Embodiments, wherein impurities in the composition are no more than about or about 17%.

370. The composition of any one of the preceding Embodiments, wherein impurities in the composition are no more than about or about 16%.

371. The composition of any one of the preceding Embodiments, wherein impurities in the composition are no more than about or about 15%.

372. The composition of any one of the preceding Embodiments, wherein impurities in the composition are no more than about or about 14%.

373. The composition of any one of the preceding Embodiments, wherein impurities in the composition are no more than about or about 13%.

374. The composition of any one of the preceding Embodiments, wherein impurities in the composition are no more than about or about 12%.

375. The composition of any one of the preceding Embodiments, wherein impurities in the composition are no more than about or about 110%.

376. The composition of any one of the preceding Embodiments, wherein impurities in the composition are no more than about or about 10%.

377. The composition of any one of Embodiments 364-376, wherein the impurities are measured by IP-RP-UPLC using area % at 260 nm.

378. The composition of any one of Embodiments 364-377, wherein the impurities are measured by IP-RP-UPLC using area % at 260 nm and the Set A parameters.

379. The composition of any one of Embodiments 364-378, wherein the impurities are measured by an IP-RP-UPLC composition for purity as described herein.

380. The composition of any one of the preceding Embodiments, wherein stereochemical purity of WVE-003 is about 80% or more.

381. The composition of any one of the preceding Embodiments, wherein stereochemical purity of WVE-003 is about 83% or more.

382. The composition of any one of the preceding Embodiments, wherein stereochemical purity of WVE-003 is about 80%-85%.

383. The composition of any one of the preceding Embodiments, wherein stereochemical purity is assessed by dimer modeling.

384. The composition of any one of the preceding Embodiments, wherein the amount of WVE-003 is measured by UV at 260 nm.

385. The composition of any one of the preceding Embodiments, wherein the amount of WVE-003 is measured by UV at 260 nm and 25.0 OD/mg.

386. The composition of any one of the preceding Embodiments, wherein the WVE-003 drug substance in the composition is manufactured by a process described herein.

387. The composition of any one of the preceding Embodiments, wherein the WVE-003 drug substance in the composition is characterized by one or more methods described herein.

388. The composition of any one of the preceding Embodiments, wherein the WVE-003 drug substance in the composition is released by one or more methods described herein.

389. The composition of any one of the preceding Embodiments, wherein the WVE-003 drug substance is stored by one or more methods described herein.

390. The composition of any one of Embodiments 386-389, wherein the WVE-003 drug substance is pentadecasodium salt.

391. The composition of any one of the preceding Embodiments, wherein the composition is a WVE-003 drug product.

392. The composition of any one of the preceding Embodiments, wherein the composition is a WVE-003 drug product which is lyophilized WVE-003 pentadecasodium salt.

393. The composition of any one of the preceding Embodiments, wherein WVE-003 drug product is manufactured by a process described herein.

394. The composition of any one of the preceding Embodiments, wherein WVE-003 drug product is characterized by one or more methods described herein.

395. The composition of any one of the preceding Embodiments, wherein WVE-003 drug product is released by one or more methods described herein.

396. The composition of any one of the preceding Embodiments, wherein WVE-003 drug product is stored by one or more methods described herein.

397. The composition of any one of the preceding Embodiments, wherein the composition is manufactured by a process described herein.

398. The composition of any one of the preceding Embodiments, wherein a pharmaceutical composition is characterized by one or more methods described herein.

399. The composition of any one of the preceding Embodiments, wherein a pharmaceutical composition is released by one or more methods described herein.

400. The composition of any one of the preceding Embodiments, wherein a pharmaceutical composition is stored by one or more methods described herein.

401. The composition of any one of the preceding Embodiments, wherein about is ±10%,

402. The composition of any one of the preceding Embodiments, wherein about is ±2%.

403. The composition of any one of the preceding Embodiments, wherein about is ±3%.

404. The composition of any one of the preceding Embodiments, wherein about is ±4%.

405. The composition of any one of the preceding Embodiments, wherein about is ±5%,

406. The composition of any one of the preceding Embodiments, wherein about is ±6%.

407. The composition of any one of the preceding Embodiments, wherein about is ±7%.

408. The composition of any one of the preceding Embodiments, wherein about is ±8%.

409. The composition of any one of the preceding Embodiments, wherein about is ±9%.

410. The composition of any one of the preceding Embodiments, wherein about is ±10%,

411. A vial comprising a composition of any one of the preceding Embodiments.

412. The vial of Embodiment 411, wherein the vial is filled with an inert gas.

413. The vial of Embodiment 411, wherein the vial is filled with nitrogen.

414. A syringe comprising a composition of any one of the preceding Embodiments.

415. The syringe of Embodiment 414, wherein the composition is a liquid composition wherein WVE-003 is dissolved in aCSF.

416. The syringe of Embodiment 414, wherein the composition is a liquid composition wherein WVE-003 pentadecasodium salt is dissolved in aCSF.

417. The syringe of any one of Embodiments 414-416, wherein the volume of the liquid composition is 20 mL.

418. The syringe of any one of Embodiments 414-417, wherein the syringe contains a WVE-003 dose of described in any one of the preceding Embodiments.

419. A method for manufacturing a WVE-003 composition according to a method described in the specification.

420. The method of Embodiment 419, comprising utilizing IP-RP-UPLC to assess purity and/or impurities in the manufactured WVE-003 composition and release the preparation if the purity and/or impurities meet certain criteria.

421. The method of any one of Embodiments 419-420, wherein the composition is a drug substance.

422. The method of any one of Embodiments 419-420, wherein the composition is a drug product.

423. A method for releasing a WVE-003 preparation, comprising utilizing IP-RP-UPLC to assess purity and/or impurities in the WVE-003 preparation and release the preparation if the purity and/or impurities meet certain criteria.

424. A method for assessing purity of WVE-003 utilizing IP-RP-UPLC.

425. The method of any one of Embodiments 420-424, wherein the IP-RP-UPLC utilized one or more parameters described in the specification.

426. The method of any one of Embodiments 420-424, wherein the IP-RP-UPLC utilized one or more parameters of Set A.

427. The method of any one of Embodiments 420-424, wherein the IP-RP-UPLC utilized the Set A parameters.

428. The method of any one of Embodiments 420-427, wherein stereochemical identity of WVE-003 is confirmed by IP-RP-UPLC.

429. A method for confirming stereochemical identity of WVE-003 utilizing IP-RP-UPLC.

430. The method of any one of Embodiments 420-429, wherein stereochemical identity of WVE-003 is confirmed by IP-RP-UPLC according to Set B parameters.

431. The method of any one of Embodiments 420-429, wherein stereochemical identity of WVE-003 is confirmed by an IP-RP-UPLC method for stereochemical identity as described herein.

432. The method of any one of Embodiments 420-431, wherein the composition is any one of the preceding Embodiments.

433. A compound, oligonucleotide, composition, method, process, use, dose or dosage regimen described in the specification.

### EXEMPLIFICATION

The foregoing has been a description of certain non-limiting embodiments of the disclosure. Accordingly, it is to be understood that embodiments of the disclosure herein described are merely illustrative of applications of principles of the disclosure. Reference herein to details of illustrated embodiments is not intended to limit the scope of any claims.

Functions and advantage of certain embodiments of the present disclosure may be more fully understood from the examples described below. The following examples are intended to illustrate certain benefits of such embodiments.

### Example 1. WVE-003 Demonstrates Suitable Properties and Activities for Human Administration to Treat Huntington's Disease

In some embodiments, nonclinical program evaluating WVE-003 encompasses in vitro and/or in vivo studies of primary and secondary pharmacology, Good Laboratory Practice (GLP)-compliant in vivo safety pharmacology, PK and general toxicity studies in the rodent and cynomolgus monkey, and in vitro and in vivo genotoxicity studies.

WVE-003 target engagement has been confirmed in in vivo studies using a bacterial artificial chromosome (BAC)-mediated transgenic mouse model (BACHD) modified to include the human mHTT bacteria artificial chromosome transgene with the A genotype on SNP3. Among other things, cynomolgus monkey cortex PK and pharmacodynamic (PD) in the BACHD mouse data supported administration of WVE-003 for treating Huntington's disease in human.

Evaluation of potential off-target effects, e.g., in silico assessments of off-target homologies, in vitro assessments of immunostimulatory effects, e.g., activation of human and mouse toll-like receptor (TLR)3 and TLR9 signaling using a human reporter assay and cytokine release from human and cynomolgus monkey peripheral blood mononuclear cells (PBMCs) and mouse and rat splenocytes, etc., standard safety pharmacology studies in rats and cynomolgus monkeys, etc., are performed. In various cases, WVE-003 demonstrated suitable safety profiles.

Absorption, distribution, and metabolism of WVE-003 were characterized in a series of in vitro and/or in vivo studies after IT, intracerebroventricular (ICV), or subcutaneous (SC) administration. In some embodiments, a Meso Scale Discovery (MSD) assay coupled with electrochemiluminescence (ECL) detection was developed for quantification in plasma, CSF, and/or tissues for all studies, with the exception of the BACHD mouse studies, which utilized a sandwich hybridization enzyme-linked immunosorbent assay (ELISA) method, and the single-dose non-GLP monkey PK study, which utilized a liquid-chromatography-high resolution mass spectrometry (LC-HRMS) method. In some embodiments, concentrations of WVE-003 in plasma, CSF, and/or tissue (spinal cord, brain, and kidney) have been evaluated following repeat-dose administration in transgenic mice, single- or repeat-dose administration in CD-1 mice (ICV and SC administration), single-dose IT administration in Sprague Dawley rats, and single- or repeat-dose IT administration in cynomolgus monkeys. In some embodiments, distribution of WVE-003 was further evaluated in in vitro protein plasma and CSF binding studies. In some embodiments, metabolites were characterized from the cortex and kidney in the BACHD mouse model and spinal cord tissue in the cynomolgus monkey. Based on PK profiles in cortex tissue in the cynomolgus monkey and PD parameters derived from the BACHD mouse, mHTT knockdown was projected in humans. In various cases, PK/PD data supported the use of WVE-003 for treating Huntington's disease in human subjects.

Among other things, WVE-003 has been assessed to support administration to humans in consideration of relevant protocols and guidance, including in Sprague Dawley rats and cynomolgus monkeys (Macaca fascicularis) as rodent and non-rodent species, respectively, for safety evaluation of WVE-003 in non-GLP dose range-finding studies with IT administration and single- and repeat-dose GLP toxicity studies. In some embodiments, GLP toxicity studies in the cynomolgus monkey utilized the IT route of administration, which is the intended route of administration for planned clinical studies. Toxicity was also investigated using single ICV and repeat SC administration routes in CD-1 mice. In some embodiments, the genotoxic potential of WVE-003 has been assessed in standard GLP in vitro and in vivo studies. Results supported the use of WVE-003 for treating Huntington's disease in human subjects.

In some embodiments, in the toxicity studies in the rat and monkey, WVE-003 was administered via direct IT injection. IT injection was also used in the subchronic study in monkeys. In some embodiments, toxicity studies were appropriately designed for assessment of systemic and local toxicity after IT administration, as well as reversibility of any effects. Results supported the use of WVE-003 for treating Huntington's disease in human subjects.

The in vitro and in vivo primary pharmacology of WVE-003 have been evaluated to confirm target engagement and selectivity for mutant huntingtin (mHTT). In some embodiments, in vitro activity of WVE-003 was confirmed in wild type (WT) induced pluripotent stem cell (iPSC) neurons that have the SNP3 A variant on both alleles. In some embodiments, a clear dose response in knockdown of HTT mRNA was observed with a calculated half-maximal inhibitory concentration (IC₅₀) of 1.36 μM. In some embodiments, in vitro allele selectivity was confirmed in Huntington's disease (HD) patient-derived iPSC neurons that have the SNP3 A variant on the allele of cytosine-adenine-guanine (CAG) expansion and the G variant on the other allele. In some embodiments, Selective reduction of the mHTT mRNA (up to 54% at 20 M) was observed, while the HTT mRNA from the normal allele only decreased 20%. The in vitro selectivity was also confirmed in an RNase H biochemical assay. In vivo studies confirming target engagement were conducted using a bacterial artificial chromosome (BAC)-mediated Huntington's disease (BACHD) transgenic mouse model. This model was developed to include the human mHTT bacterial artificial chromosome transgene with the A variant of SNP3 in exon 57. In addition, secondary pharmacology studies and safety pharmacology studies were conducted to identify potential off-target toxicities.

In some embodiments, to assess the dose-dependent activity of WVE-003 in vivo, BACHD mice received 3 injections through an ICV cannula of phosphate-buffered saline (PBS) and different dose levels of WVE-003 (12.5 μg, 25 μg, 50 μg, or 100 μg) on Day 1, Day 3, and Day 5 (8 per group). Mice were euthanized on Day 15 (2 weeks after the first injection). Cortex and striatum tissues were collected for HTT mRNA expression by qPCR. HTT transcripts were significantly reduced in the cortex and striatum tissues of mice that received 3 doses of >25 μg WVE-003 In the cortex, HTT transcripts were reduced by 18.9% (p=0.0048), 38.4% (p<0.0001), and 56.9% (p<0.0001) in mice that received 3 doses of 25 μg, 50 μg, and 100 μg of WVE-003, respectively. In the striatum, HTT transcripts were reduced by 21.0% (p=0.0104), 37.9% (p<0.0001), and 54.2% (p<0.0001) in mice that received 3 doses of 25 μg, 50 μg, and 100 μg of WVE-003, respectively. No significant changes were observed in mice that received 3 doses of 12.5 μg WVE-003.

In another study, mice received 3 ICV cannula injections of PBS (n=4) or 100 μg of WVE-003 (n=8) on Day 1, Day 3, and Day 5, and were euthanized on Day 15. Knockdown of HTT transcripts was observed with 62.4% reduction in the cortex and 69.4% reduction in the striatum (both p<0.0001).

In another study, BACHD transgenic mice received 3 ICV cannula injections of PBS (n=6) or 100 μg of WVE-003 (n=8) on Day 1, Day 3, and Day 5. Mice were euthanized on Day 15 (2 weeks), Day 29 (4 weeks), Day 57 (8 weeks), or Day 85 (12 weeks) after the first injection. Cortex and striatum tissues were collected for HTT mRNA expression by qPCR. Huntingtin transcripts were significantly reduced in cortex and striatum tissues in mice treated with WVE-003 at each time point tested (p<0.0001). Knockdown of HTT transcripts ranging from approximately 50.1% to 63.8% was observed in cortex tissue and approximately 41.4% to 60.9% was observed in the striatum, similar to and consistent with other studies. No significant changes were observed with PBS treatment.

In another study, BACHD transgenic mice (8/group) received 3 ICV cannula injections of PBS or 12.5, 25, 50, or 100 μg of either WVE-003 or another HTT-targeted comparator oligonucleotide on Day 1, Day 3, and Day 5. Mice were euthanized on Day 29 (4 weeks) after the first injection. Cortex and striatum tissues were collected for PK and HTT mRNA expression by qPCR. Huntingtin transcripts were reduced in the cortex and striatum tissues in mice treated with WVE-003 compared to PBS control at 4 weeks. HTT transcripts were 80%, 50%, 42%, and 41% that of PBS at 3 doses of 12.5, 25, 50, and 100 pg, respectively, in the cortex (p<0.01 to p<0.001 at doses >25 pg) and 73%, 70%, 69%, and 47% that of PBS at 12.5, 25, 50, and 100 μg, respectively, in the striatum (p<0.01 at 100 pg). No knockdown of HTT transcript was observed with PBS treatment.

In another study, BACHD transgenic mice (n=7 or 8) received 3 ICV cannula injections of PBS or 100 μg WVE-003 on Day 1, Day 3, and Day 5. Mice were euthanized on Day 15 (2 weeks), Day 29 (4 weeks), and Day 85 (12 weeks) after the first injection. Cortex and striatum tissues were collected for PK and HTT mRNA expression by qPCR. Huntingtin transcripts were significantly reduced in cortex and striatum tissues in mice treated with WVE-003 at each time point. HTT transcripts were 40%, 58%, and 62% that of PBS at 2, 4, and 12 weeks, respectively, in the cortex (p<0.05) and 31%, 32%, and 51% that of PBS at 2, 4, and 12 weeks, respectively, in the striatum (p<0.0001). No knockdown of HTT transcript was observed with PBS treatment.

Bioinformatics screening study identified certain sequences that are homologous to WVE-003 target sequence, e.g., 18/20 matches, 16/20 matches etc. All of the identified off-target matches possessed substantially reduced binding free energies compared with the on-target HTT binding site, suggesting that off-target effects if any will be minimal. When assessed in motor neuron cells, effects on such off-targets were much lower than on targeted HTT. In some embodiments, for a potential off-target, no significant and/or no dose-dependent knockdown on mRNA levels was observed. It was concluded that WVE-003 would have little if any off-target effects in view of WVE-003 tissue concentration and/or half-life.

Data from TLR3 and TLR9 activation assays suggested the risk of WVE-003 inducing an immune response via TLRs is low.

Effects of WVE-003 on cytokine and chemokine production in isolated cells from multiple species were assessed. Differences compared to PBS control were observed but were considered to be toxicologically relevant. It was concluded the risk for an inflammatory response was low.

A cardiovascular and respiratory assessment following intravenous injection administration of WVE-003 to monkeys observed no effect at 20 mg/animal. In some embodiments, no-observed-adverse-effect level (NOAEL) for neurological effects of WVE-003 in rats was considered to be 0.9 mg.

In some embodiments, pharmacokinetics (PK) of WVE-003, including absorption, distribution, and metabolism, was characterized in transgenic BACHD mice, CD-1 mice, Sprague Dawley rats, and/or cynomolgus monkeys following a single dose and/or repeat doses. In addition, target engagement in patients was projected using a PK/pharmacodynamics (PD) model built on data from the BACHD transgenic mouse and the cynomolgus monkey described herein.

In some embodiments, WVE-003 concentrations in various matrices were measured using a hybridization MSD assay coupled with ECL detection, sandwich hybridization enzyme-linked immunosorbent assay (ELISA), or a liquid chromatography-high-resolution mass spectrometry (LC-HRMS) assay. In some embodiments, metabolism of WVE-003 was characterized using an LC-HRMS assay.

Various PK/PD results support administration of WVE-003 to human subjects for treating Huntington's disease.

In totality, data support that WVE-003 has the potential to be an effective disease-modifying treatment, with a manageable safety profile, to address the significant unmet need experienced by patients with HD.

### Example 2. Clinical Studies of WVE-003

Safety and efficacy of WVE-003 for treating human subjects suffering from Huntington's disease are assessed in clinical trials. In some embodiments, a trial or a portion thereof is performed as described in a clinical trial with a ClinicalTrials.gov Identifier: NCT05032196.

In some embodiments, WVE-003 is assessed in a multicenter, randomized, double-blind, placebo-controlled, study of WVE-003 administered intrathecally in patients with Huntington's disease. In some embodiments, a clinical study is or comprises Phase 1b/2a. In some embodiments, a clinical study is or comprises Phase 2b. In some embodiments, a clinical study is or comprises Phase 3. In some embodiments, safety, tolerability, PK, PD, and/or clinical effects of WVE-003 in patients with HD are assessed.

In some embodiments, a study is or comprises a Phase 1b/2a multicenter, randomized, double-blind, placebo-controlled study to evaluate the safety, tolerability, PK, pharmacodynamics (PD), and clinical effects of WVE-003 in adult patients with early-manifest Huntington's disease (HD) who carry a targeted single nucleotide polymorphism (SNP) rs362273 (SNP3). In some embodiments, Period 1 of a study evaluates single ascending doses (SAD) of WVE-003. In some embodiments, Period 2 evaluates multiple ascending doses (MAD) of WVE-003. In some embodiments, Period 2 does not be initiated until nonclinical data supportive of multiple dosing is submitted to and approved by the competent authority(ies).

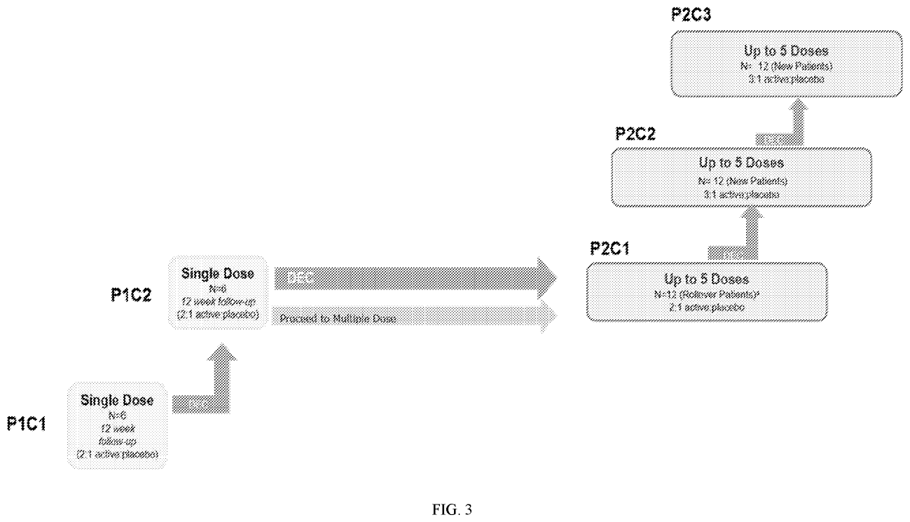

Patients may participate in Period 1 (SAD) and Period 2 (MAD) or Period 2 (MAD) only. In a study, all patients enrolled in Period 1 cohorts have the opportunity to receive multiple doses in Period 2. Cohorts in both Period 1 and Period 2 are enrolled and dosed in a sequential manner. Subsequent cohorts are not initiate until the requirements for dose escalation are met. A schematic of the overall study design and timing is provided in FIG. 3.

An open-label extension study may be conducted, providing treatment for up to 2 years for patients who complete a Phase 1b/2a study.

### Period 1

Period 1 evaluates SAD cohorts of WVE-003. The Period 1 dose cohorts are as follows: Period 1 Cohort 1 (P1C1): 6 patients (2:1 active: placebo); 30 mg. P1C2: 6 patients (2:1 active: placebo). In some embodiments, P1C2 may not be conducted and a study may proceed directly to Period 2. In some embodiments, subsequent SAD cohorts may be conducted. All patients are assessed for safety, PK, PD, and clinical effects through a follow-up period, e.g., a 12-week follow-up period.

### Period 2

Dose levels for Cohort 2 are determined based on Period 1 data, and in some embodiments, with repeat-dose toxicity data from nonclinical studies. Doses are administered on Weeks 0, 2, and 4 and monthly thereafter (5 total doses). The Period 2 cohorts are as follows:
P2C1: 6 rollover patients from P1C1 and 6 rollover patients (2:1 active: placebo) from P1C2 or 6 new patients (12 patients total; 2:1 active: placebo)
P2C2: 12 new patients (3:1 active: placebo)
P2C3: 12 new patients (3:1 active: placebo)

All patients from Period 1 have the opportunity to roll into Period 2 (P2C1) and receive 5 additional doses of WVE-003 or placebo. Patients are not re-randomized and continue to receive active or placebo based on Period 1 treatment assignment. If P1C2 is not conducted, then 6 new patients (2:1 active: placebo) are enrolled in P2C1 to ensure this cohort consists of 12 total patients. In some cases, Period 2 are not conducted unless the dose level has already been evaluated in Period 1.

Patients in P2C2 and P2C3 can receive up to 5 doses. There are up to 12 new patients allocated 3:1 active: placebo in both cohorts. Additional cohorts with up to 12 patients each may be added.

Patient monitoring is implemented for safety and/or efficacy, including assessment of motor function, weakness, or fatigue postdose. In addition, formal physical examinations targeting the neurological system, with special attention to the motor system, upper and lower extremity measures of strength, tone, and reflexes, as well as ambulation, is performed postdose. The following effects are monitored: liver function, kidney function, complement and cytokine activation, and other potential class effects including thrombocytopenia, aPTT prolongation (plasma aPTT), coagulation etc. Inflammatory markers, aPTT, platelet counts, LFTs, and renal biomarkers can also be monitored.

WVE-003 drug product is supplied as a lyophilized powder for reconstitution and dilution for solution for injection. Artificial CSF (aCSF; placebo and diluent for WVE-003) is supplied in single-use vials. WVE-003 is diluted with aCSF and administered in a volume of 20 mL.

In some embodiments, outcome measures comprises safety and/or efficacy outcomes. In some embodiments, outcome measures comprises proportion of patients with adverse events (AEs). In some embodiments, outcome measures comprises maximum concentration (Cmax) of WVE-003 in plasma. In some embodiments, outcome measures comprises area under the plasma concentration time curve for WVE-003 from time 0 to last quantifiable concentration (AUC0-t). In some embodiments, outcome measures comprises terminal half-life (t1/2) of WVE-003 in cerebrospinal fluid (CSF).

Various time frames may be utilized for outcome measures. In some embodiments, a time frame is from Day 1 through the end of a study. In some embodiments, a time frame is 36 weeks or more.

In some embodiments, a patient has A variant of SNP3 on the same allele as the pathogenic CAG triplet expansion.

In some embodiments, eligible criteria are or comprise the following
1. Presence of the A variant of SNP3 on the same allele as the pathogenic CAG triplet expansion (36 or more CAG repeats)
2. Ambulatory, male or female patients aged ≥25 to ≤60 years
3. Clinical diagnostic motor features of HD, defined as Unified Huntington's Disease Rating Scale (UHDRS) Diagnostic Confidence Score=4
4. UHDRS Total Functional Capacity Scores ≥9 and ≤13

In some embodiments, the following exclusion criteria are applied:
1. Malignancy or received treatment for malignancy, other than treated basal cell or squamous cell carcinoma of the skin, within the previous 5 years
2. Received any other study drug, including an investigational oligonucleotide, within the past 1 year or 5 half-lives of the drug, whichever is longer, with the exception of the following: a. Received WVE-120101 or WVE-120102 within the last 3 months
3. Implantable CNS device that may interfere with ability to administer study drug via lumbar puncture or undergo MRI scan
4. Inability to undergo brain MRI (with or without sedation)
5. Bone, spine, bleeding, or other disorder that exposes the patient to risk of injury or unsuccessful lumbar puncture
6. Previously received tominersen

In some embodiments, an interval between a dose and its immediate following dose is about 1, 2, 3, 4, 5, 6, 7, 8, 9, or 10 weeks, or about 1, 2, 3, 4, 5, 6, 7, 8, 9, 10, 11, 12 or more months. In some embodiments, dose intervals are the same. In some embodiments, dose intervals are different. In some embodiments, dose intervals of the first one or more doses may be shorter. In some embodiments, an interval is about 2 weeks. In some embodiments, an interval is about 4 weeks. In some embodiments, an interval is about 6 weeks. In some embodiments, an interval is about 8 weeks. In some embodiments, an interval is about 1 month. In some embodiments, an interval is about 2 months. In some embodiments, an interval is about 3 months. In some embodiments, an interval is about 4 months. In some embodiments, an interval is about quarterly.

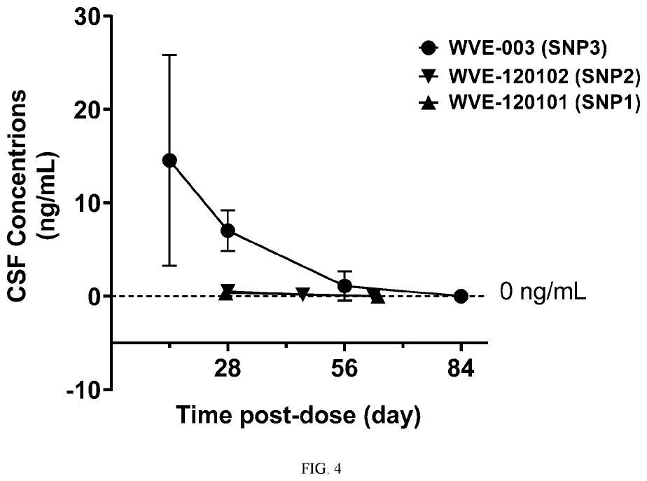

As demonstrated in FIG. 4, dose of 30 mg WVE-003 is confirmed to provide greatly improved PK profiles compared to previously assessed oligonucleotides, e.g., WVE-120101 and WVE-120102, and is expected to provide pharmacologic activity. This dose level is associated with a 5.6-fold safety margin based on the HED of the single-dose NOAEL observed in monkey and a 16.8-fold safety margin based on the HED of the single-dose NOAEL observed in rats. This dose is selected to adequately balance the risk/benefit for patients. Those skilled in the art will appreciate that other doses, either lower or higher than 30 mg, may also be utilized in accordance with the present disclosure. Higher doses are expected to provide greater mHTT mRNA knockdown and potentially more therapeutic benefits associated with mHTT reduction.

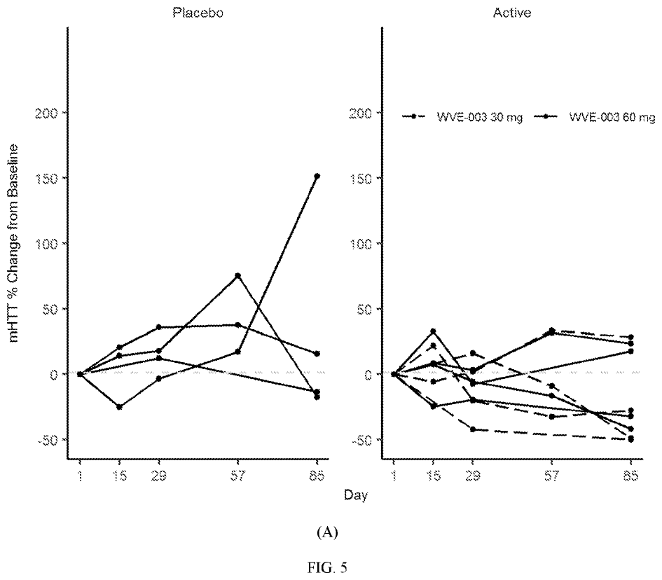

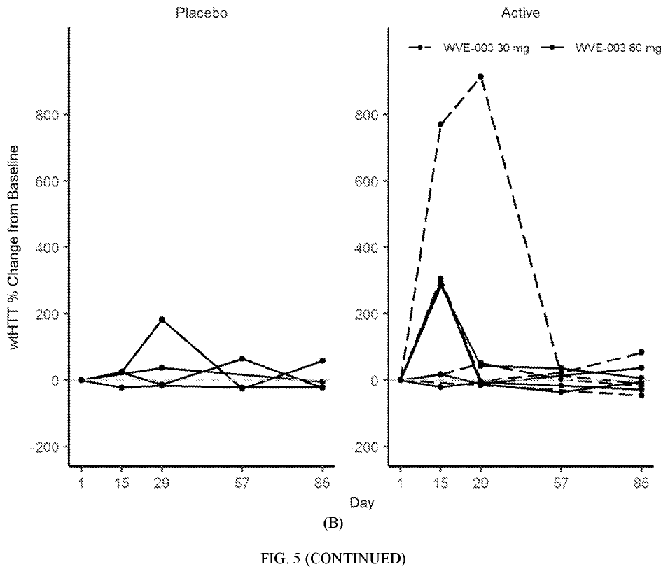

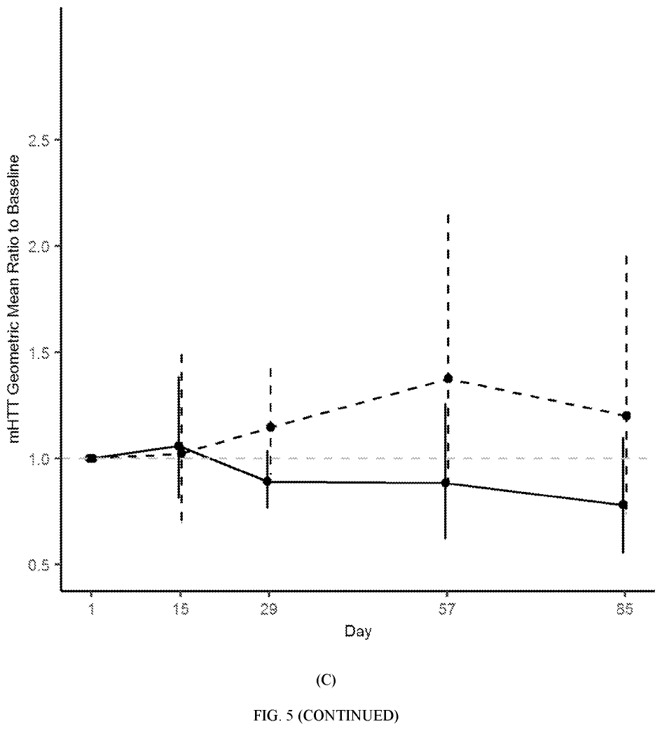

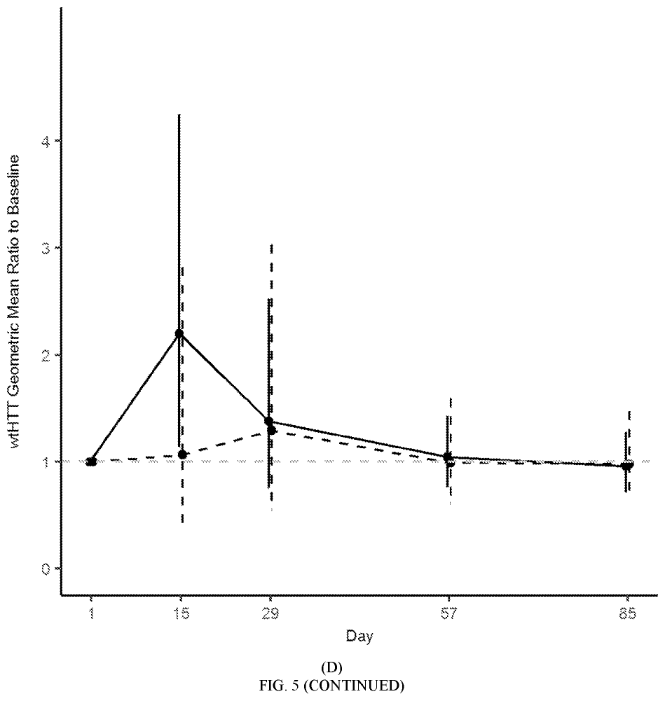

Further, as confirmed in FIG. 5, reductions of mHTT protein in cerebrospinal fluid (CSF) after study participants received either a single 30 or 60 mg dose of WVE-003 was observed with wild-type huntingtin (wtHTT) protein preserved through day 85. Among other things, these data confirm that WVE-003 can selectively reduce toxic mHTT protein while avoid targeting healthy, wild-type huntingtin protein, thereby preserving the beneficial effects of the wild-type huntingtin protein including in the central nervous system. Data also demonstrated that WVE-003 is safe and tolerated. No patient discontinued. Variable technologies for assessing levels of huntingtin proteins are available (e.g., those described in WO 2022/046723) and may be utilized in accordance with the present disclosure.

In an assessment, included are eighteen (18) early manifest Huntington's disease patients (30 mg single dose, n=4; 60 mg single dose, n=4; 90 mg, n=4; placebo, n=6). Participants enrolled at 30 mg, 60 mg and placebo had adequate follow-up to day 85 for biomarker analysis. At a time of analysis, none of the participants dosed with 90 mg had reached day 85 so this cohort is not included in the biomarker analysis. Reductions in mHTT were observed in both 30 and 60 mg single dose treatment groups (30 mg, n=4 patients; 60 mg, n=4 patients), with a 22% mean reduction (30% median reduction). Key observations include:
Single doses of WVE-003 up to 90 mg appeared generally safe and well-tolerated
   Adverse events (AEs) were balanced across treatment groups, including placebo, and all were mild to moderate in intensity
   No serious adverse events (SAEs) were observed
   No participants discontinued the study
Among participants in the 30 and 60 mg WVE-003 cohorts, the mean reduction in CSF mHTT from baseline was 22% (median reduction 30%) at 85 days following a single dose
   The difference in the mean reduction in CSF mHTT compared to placebo was 35% at 85 days post-single dose
   For these analyses, the 30 and 60 mg single dose cohorts were pooled as there was no apparent dose response between these two cohorts based on this data set at this time point. Dose response in other cohorts e.g., expanded single dose cohorts will be evaluated
In the 30 and 60 mg cohorts, wtHTT protein was preserved, which appears consistent with allele-selectivity
Increases in neurofilament light chain (NfL) from baseline were observed in some patients. Applicant will continue to monitor trends in NfL as SELECT-HD advances
There were no clinically meaningful elevations in CSF white blood cell counts or protein that would indicate inflammation in the CNS
No meaningful changes in clinical outcome measures (e.g., eUHDRS, TFS, TMS, etc.) were observed as the dataset and duration were not sufficient to assess clinical effects Certain results are presented in FIG. 5 as examples.

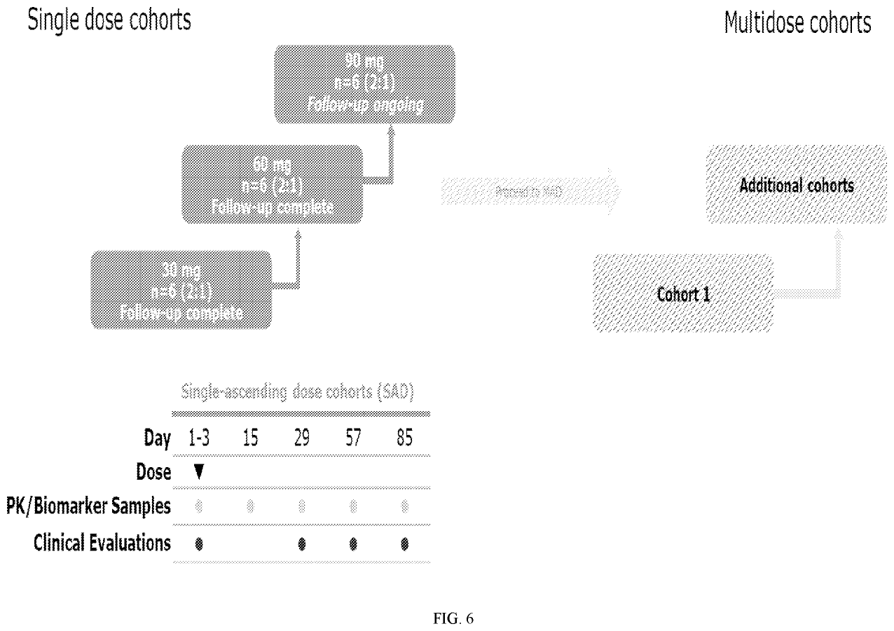

As described herein, SELECT-HD was designed as an adaptive trial, and can be adapted, among other things, to expand single dose cohorts (e.g., see FIG. 6). Additional single and multidose data may be used to optimize dose level and frequency. SELECT-HD was designed to deliver an early indication of target engagement so that dose can be rapidly optimize. Various objectives may be included, e.g., safety, tolerability, PK profiles (plasma, CSF, etc.), levels of mHTT, wtHTT and/or NfL in CSF, clinical assessments (in some embodiments, including MRI).

In some embodiments, the present disclosure provides additional regimens that can further increase knockdown, e.g., with repeat dosing, in some embodiments, with quarterly or less frequent dosing. Among other things, the present disclosure demonstrates that stereodefined oligonucleotides comprising PN linkages (e.g., phosphoryl guanidine linkages such as n001), e.g., WVE-003, can enable potent and durable target engagement, in some cases, at single doses (e.g., for WVE-003, 30 mg and 60 mg). In some embodiments, duration of effects can provide dosing frequency of every two months or less. In some embodiments, duration of effects can provide quarterly or less frequent dosing. In some embodiments, inclusion criteria include >=25 to <=60 years old with the targeted SNP3 on mHTT allele. In some embodiments, subjects are >=25 to <=60 years old with the targeted SNP3 on mHTT allele. In some embodiments, subjects are >=25 to <=60 years old, with the targeted SNP3 on mHTT allele, and with early manifest Huntington's disease.

### Other Period 2

In some embodiments, Period 2 may comprise a clinical study as described below.

Dose levels for Period 2 are determined based on Period 1 data, and in some embodiments, with repeat-dose toxicity data from nonclinical studies. For example, data from animal studies confirmed that administration of an about 30 mg dose of WVE-003 about every 8 weeks would be safe for human subjects, e.g., patients. Among other things, it was observed that repeat-dose of 3 mg of WVE-003 (may be considered human equivalent dose of about 34 mg) administered once every 8 weeks in non-human primates were safe. Further, administration of 3 mg of WVE-003 once every 8 weeks in non-human primates confirmed useful brain concentrations (e.g., in putamen, in cortex, e.g., frontal cortex) of WVE-003 were achieved. Period 2 data from dosing with 30 mg, 60 mg, or 90 mg WVE-003 are used to determine, e.g., additional dose levels, etc.

In some embodiments, a dose is about 30 mg. In some embodiments, a dose is about 60 mg. In some embodiments, a dose is about 90 mg. In some embodiments, of multiple or all doses, each dose is about 30 mg. In some embodiments, of multiple or all doses, each dose is about 60 mg. In some embodiments, of multiple or all doses, each dose is about 90 mg. In some embodiments, doses are administered no more often than once every 8 weeks. In some embodiments, doses are administered on Weeks 0, 8, and 16.

All patients from Period 1 have the opportunity to roll into Period 2 (P2C1) and receive 3 additional doses of WVE-003 or placebo. Patients are not re-randomized and continue to receive active or placebo based on Period 1 treatment assignment. Patients are rolled into the current Period 2 cohort, irrespective of Period 1 dose level. In some embodiments, new patients are added to Period 2. New patients in Period 2 receive up to 3 doses of WVE-003 or placebo.

Additional cohorts (e.g., P2C2, P2C3) are added based on data from P2C1. In some embodiments, additional cohorts receive the same dose level as P2C1. In some embodiments, additional cohorts receive an alternate dose level (e.g., a higher dose, a lower dose). In some embodiments, additional cohorts receive the number of doses as P2C1. In some embodiments, additional cohorts receive an alternate number of doses (e.g., an increased number of doses, a decreased number of doses). In some embodiments, frequency of doses administered to additional cohorts is the same as P2C1. In some embodiments, frequency of doses administered to additional cohorts is increased or decreased. In some embodiments, additional cohorts receive doses every 8 weeks (e.g., on weeks 0, 8, 16). In some embodiments, additional cohorts receive doses every 12 weeks (e.g., on weeks, 0, 12). Selected dose levels will not increase by >3-fold the previous dose level and will not exceed the highest dose level evaluated in Period 1.

Patients in Period 2 are assessed at least once every 4 weeks. Assessments include safety assessments, pharmacokinetic assessments, and pharmacodynamics assessments as described herein.

Additional clinical trials can also be designed and conducted by those skilled in the art in accordance with the present disclosure to assess safety and efficacy of WVE-003.

1. A method for treating a subject who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, about 60 mg, about 90 mg, about 120 mg, about 150 mg, about 160 mg, or about 168 mg, such that progression of Huntington's disease in the subject is delayed, and/or the onset of Huntington's disease is delayed, and/or the severity of a symptom of Huntington's disease is reduced.

2. A method for treating Huntington's disease in a subject who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, about 60 mg, about 90 mg, about 120 mg, about 150 mg, about 160 mg, or about 168 mg, such that disease progression in the subject is delayed, and/or the onset of Huntington's disease is delayed, and/or the severity of a symptom of Huntington's disease is reduced.

3. A method of delaying the onset of and/or reducing the severity of a symptom of Huntington's disease in a subject with Huntington's disease who has a mutant HTT gene comprising a mutation that is amenable to an allele-specific knockdown of the mutant HTT gene, wherein the method comprises the step of administering to the subject WVE-003 (or a salt form thereof) at a dose of about 30 mg, about 60 mg, about 90 mg, about 120 mg, about 150 mg, about 160 mg, or about 168 mg,.

4. A method for treating Huntington's disease, comprising administering to a subject suffering therefrom WVE-003 (or a salt form thereof), wherein WVE-003 is administered at a dose of about 30 mg, about 60 mg, about 90 mg, about 120 mg, about 150 mg, about 160 mg, or about 168 mg, and wherein the subject has a HTT allele that comprises an expanded CAG repeat region and is fully complementary to the base sequence of WVE-003.

5. A method for treating Huntington's disease, comprising administering to a subject suffering therefrom a pharmaceutical composition that comprises or delivers WVE-003 (or a salt form thereof), wherein WVE-003 is administered at a dose of about 30 mg, about 60 mg, about 90 mg, about 120 mg, about 150 mg, about 160 mg, or about 168 mg, and wherein the subject has a HTT allele that comprises an expanded CAG repeat region and is fully complementary to the base sequence of WVE-003.

6. A method, comprising administering to a subject WVE-003 (or a salt form thereof), wherein the subject is determined to have a genetic sequence that is the same or fully complementary to the base sequence of WVE-003, optionally wherein the subject is determined to have a genetic sequence that is or encodes an expanded CAG repeat.

7. A method, comprising administering to a subject WVE-003 (or a salt form thereof), wherein the subject is determined to have a genetic sequence that encodes a transcript that comprises an expanded CAG repeat in HTT and is fully complementary to the base sequence of WVE-003.

8. A method, comprising administering to a subject WVE-003 (or a salt form thereof), wherein the subject is determined to express a HTT transcript that comprises an expanded CAG repeat and is fully complementary to the base sequence of WVE-003.

9. The method of any one of claims 5-7, wherein the subject is determined to have a genetic sequence or transcript that is not the same or fully complementary to the base sequence of WVE-003 at rs362273, optionally wherein the genetic sequence or transcript does not contain expanded CAG repeats (or a sequence encoded thereby).

10. The method of any one of claims 5-9, wherein WVE-003 is administered at a dose of about 30 mg, about 60 mg, about 90 mg, about 120 mg, about 150 mg, about 160 mg, or about 168 mg.

11. The method of any one of the previous claims, wherein the method further comprises the step of confirming that the subject has a mutation in the HTT gene that is amenable to an allele-specific knockdown of the mutant HTT gene or a gene product thereof transcript.

12. The method of any one of the previous claims, wherein WVE-003 is administered in a salt form, optionally a sodium salt form.

13. The method of any one of the previous claims, wherein WVE-003 is formulated as a liquid formulation, optionally wherein the liquid formulation comprises WVE-003, sodium chloride and water and/or wherein the liquid formulation is reconstituted from a lyophilized preparation.

14. The method of any one of the preceding claims, wherein one or more pharmaceutically acceptable salt forms of WVE-003 are administered and/or wherein the amount of WVE-003 includes the amount of one or more pharmaceutically acceptable salt forms, each of which is independently converted to the amount of the acid form.

15. A method for treating Huntington's disease, comprising administering or delivering to a subject suffering therefrom WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for preventing Huntington's disease, comprising administering or delivering to a subject suffering therefrom WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method, comprising administering or delivering to a subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for decreasing the activity, expression, and/or level of a mutant HTT gene or its gene product in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for preferential knockdown of a repeat expansion-containing HTT RNA transcript relative to a non-repeat expansion-containing HTT RNA transcript in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for reducing level of a HTT transcript comprising CAG repeat expansion in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for reducing level of a product of a HTT transcript comprising CAG repeat expansion in a subject, comprising administering or delivering to the subject WVE-003 at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form;
wherein WVE-003 is:
mG*S mUn001R mU mGn001R mA*ST*SC*ST*SG*ST*RA*SG*SC*SA *SG*R m5Ceon001RAeoGeon001R m5Ceo*STeo, wherein:
m represents a 2′-OMe modification to a nucleoside;
*S represents a Sp phosphorothioate linkage;
m5Ceo represents 5-methyl 2′-O-methoxyethyl C;
n001R represents a Rp n001 linkage, wherein a n001 linkage has the structure of 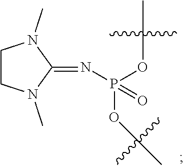 [SMILES: *OP(=O)(N=C1N(C)CCN1C)O*]
eo represents a 2′-OCH2CH2OH3 modification to a nucleoside; and
*R represents a Rp phosphorothioate linkage.

16. The method of any one of the preceding claims, wherein a dose of WVE-003 is administered in one or more forms, optionally wherein a dose of WVE-003 is administered in one or more pharmaceutically acceptable salt forms, optionally wherein one form is WVE-003 pentadecasodium salt.

17. The method of any one of the preceding claims, wherein a dose of WVE-003 is administered in a pharmaceutical composition comprising WVE-003 and a pharmaceutically acceptable carrier.

18. The method of any one of the preceding claims, wherein a dose of WVE-003 is administered in a pharmaceutical composition comprising or consisting of a WVE-003 pentadecasodium salt and a pharmaceutically acceptable carrier.

19. The method of any one of the preceding claims, wherein each dose of WVE-003 is independently administered in one or more pharmaceutically acceptable salt forms, optionally wherein one form is WVE-003 pentadecasodium salt.

20. The method of any one of the preceding claims, wherein each dose of WVE-003 is independently administered in a pharmaceutical composition comprising WVE-003 and a pharmaceutically acceptable carrier.

21. The method of any one of the preceding claims, wherein each dose of WVE-003 is independently administered in a pharmaceutical composition comprising or consisting of a WVE-003 pentadecasodium salt and a pharmaceutically acceptable carrier.

22. A method for treating Huntington's disease, comprising administering or delivering to a subject suffering therefrom WVE-003 pentadecasodium salt: 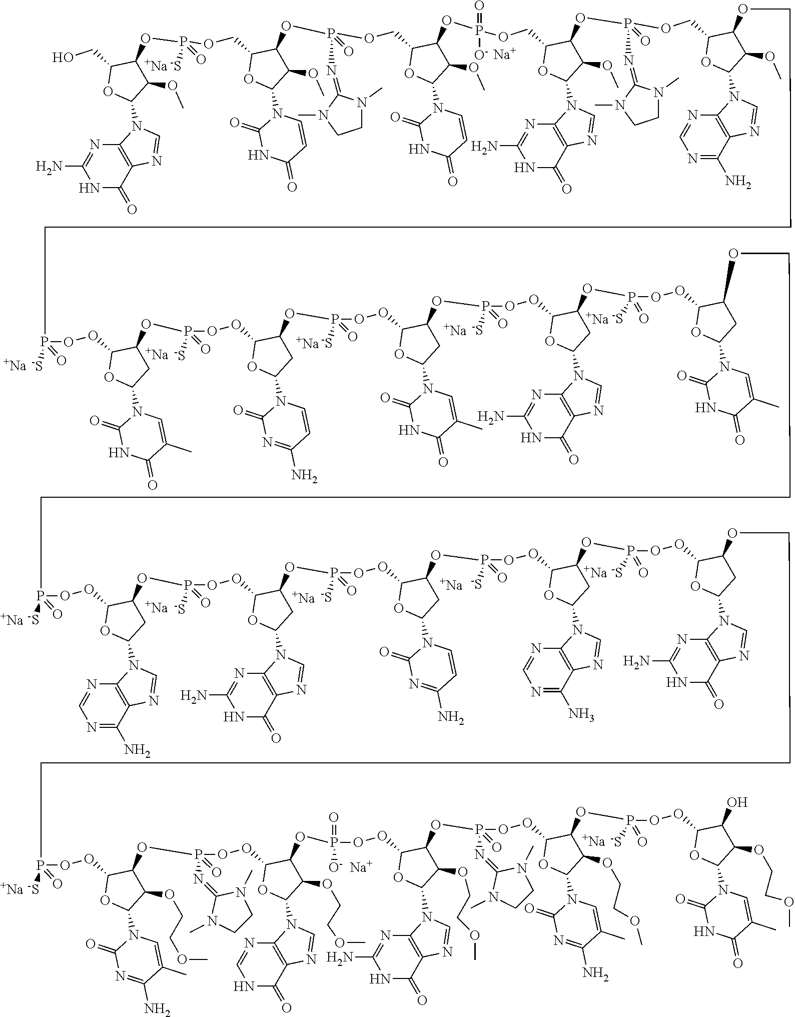
at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

23. A method for preventing Huntington's disease, comprising administering or delivering to a subject suffering therefrom WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

24. A method, comprising administering or delivering to a subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for decreasing the activity, expression, and/or level of a mutant HTT gene or its gene product in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for preferential knockdown of a repeat expansion-containing HTT RNA transcript relative to a non-repeat expansion-containing HTT RNA transcript in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for reducing level of a HTT transcript comprising CAG repeat expansion in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form; or
a method for reducing level of a product of a HTT transcript comprising CAG repeat expansion in a subject, comprising administering or delivering to the subject WVE-003 pentadecasodium salt at a dose equivalent to about 10-200 mg (e.g., about 10-200 mg, about 10-190 mg, about 10-180 mg, about 10-170 mg, about 10 mg, about 20 mg, about 30 mg, 40 mg, about 50 mg, about 60 mg, about 70 mg, about 80 mg, 90 mg, about 100 mg, about 110 mg, about 120 mg, about 130 mg, about 140 mg, about 150 mg, about 160 mg, or about 168 mg) WVE-003 free acid form.

25. The method of any one of the preceding claims, wherein the product is a polypeptide, optionally a polypeptide comprising expanded poly-Q.

26. The method of any one of the preceding claims, wherein two or more doses, e.g., 2, 3, 4, 5, 6, 7, 8, 9, 10, 11, 12, 13, 14, 15, 16, 17, 18, 19, 20 or more doses are administered.

27. The method of any one of the preceding claims, wherein a dose of WVE-003 pentadecasodium salt is administered in a pharmaceutical composition comprising or consisting of WVE-003 pentadecasodium salt and a pharmaceutically acceptable carrier.

28. The method of any one of the preceding claims, wherein each dose of WVE-003 pentadecasodium salt is independently administered in a pharmaceutical composition comprising or consisting of WVE-003 and a pharmaceutically acceptable carrier.

29. The method of any one of claims 22-28, wherein the pharmaceutically acceptable carrier is aCSF.

30. The method of any one of the preceding claims, wherein the pharmaceutical composition has a pH of about 6-8, optionally wherein the pharmaceutical composition has a pH of about 6.4-7.2 or about 7.3 or about 7.4.

31. The method of any one of claims 22-29, wherein a dose is equivalent to about 10, 20, 30, 40, 50, 60, 70, 80, 90, 100, 110, 120, 130, 140, 150, 160, or 170 mg WVE-003 free acid form.

32. The method of any one of claims 22-29, wherein each dose is independently equivalent to about 10, 20, 30, 40, 50, 60, 70, 80, 90, 100, 110, 120, 130, 140, 150, 160, or 170 mg WVE-003 free acid form.

33. The method of any one of the preceding claims, wherein two or more (e.g., 2, 3, 4, 5, 6, 7, 8, 9, 10, 11, 12, 13, 14, 15, 16, 17, 18, 19, 20 or more) consecutive doses are administered independently about every 1, 2, 3, 4, 5, 6, 7, 8, 9, 10, 11, 12 weeks, or about every 3, 4, 5, 6, 7, 8, 9, 10, 11, 12 or more months, or about every 1, 2, 3, or 4 quarters.

34. The method of any one of the preceding claims, wherein all doses are administered independently about every 1, 2, 3, 4, 5, 6, 7, 8, 9, 10, 11, 12 weeks, or about every 3, 4, 5, 6, 7, 8, 9, 10, 11, 12 or more months, or about every 1, 2, 3, or 4 quarters.

35. The method of any one of the previous claims, wherein the subject is administered WVE-003 approximately monthly for at least about 2, 4, 8, 12, 16, 24, or 48 months, the subject is administered WVE-003 approximately once every 4 weeks for at least about 8, 12, or 16 weeks, or the subject is administered WVE-003 approximately once every 8 weeks for at least about 8 or 16 weeks.

36. The method of any one of the preceding claims, wherein two or more (e.g., 2, 3, 4, 5, 6, 7, 8, 9, 10, 11, 12, 13, 14, 15, 16, 17, 18, 19, 20 or more) consecutive doses of WVE-003, each independently equivalent to about 30 mg free acid form, are administered about every 8 weeks.

37. The method of any one of the preceding claims, wherein the subject has expanded CAG repeat region in a HTT gene and/or expresses a HTT transcript comprising an expanded CAG repeat region; and/or
wherein the expanded CAG repeat region comprises 36 or more CAG repeats, optionally 40 or more CAG repeats.

38. The method of any one of the preceding claims, wherein the A variant of rs362273 is on the same allele as the expanded CAG repeat region in a HTT gene.

39. The method of any one of the preceding claims, wherein expression of mutant HTT is reduced and/or mutant HTT protein level is reduced, and/or wherein the level, expression and/or activity of a mutant HTT transcript or gene product thereof is reduced by at least about 5% or 10%.

40. The method of any one of the preceding claims, wherein level of mutant HTT transcript in cerebrospinal fluid is reduced by about 10%, 20%, 30%, 40%, 50% or more about or after about 1, 2, 3, 4, 5, 6, 7, 8, 9, 10, 11, 12, 13, 14, 15, 16, 17, 18, 19, 20 or more weeks after a first dose.

41. The method of any one of the preceding claims, wherein level of mutant HTT polypeptide in cerebrospinal fluid is reduced by about 10%, 20%, 30%, 40%, 50% or more about or after about 1, 2, 3, 4, 5, 6, 7, 8, 9, 10, 11, 12, 13, 14, 15, 16, 17, 18, 19, 20 or more weeks after a first dose.

42. The method of any one of the preceding claims, wherein level of wtHTT transcript or polypeptide is reduced by no more than about 10%, 20%, 30%, 40% or 50%.

43. The method of any one of the preceding claims, wherein the method provides reduction of level of the repeat expansion-containing HTT transcript as measured by percentage that is at least 1.1, 1.2, 1.3, 1.4, 1.5, 1.6, 1.7, 1.8, 1.9, 2, 2.5, 3, 4, 5, 6, 7, 8, 9, or 10 fold of the reduction of level of the non-repeat-expansion-containing HTT transcript as measured by percentage.

44. The method of any one of the preceding claims, wherein the reduction is about 10%, 12%, 15%, 20%, 25%, 30%, 40%, 50%, 60%, 70%, 80%, or 90% or more; and/or
wherein the reduction is assessed for an individual subject; and/or
wherein the reduction is assessed for a population of subjects, optionally wherein the population size is about 10, 20, 30, 40, 50, 60, 70, 80, 90, 100, 200, 500, 1000 or more subjects and/or wherein subjects in the population receive the same or different dosage regimen; and/or
wherein one or more cerebrospinal fluid samples are utilized for reduction assessment.

45. The method of any one of the preceding claims, wherein wild type HTT transcript level is not significantly reduced; and/or wherein wild type HTT protein level is not significantly reduced; and/or
wherein total HTT transcript level is not significantly reduced; and/or wherein total HTT protein level is not significantly reduced; and/or wherein neurofilament light chain (NfL) level in CSF is not significantly increased.

46. The method of any one of the preceding claims, wherein assessment is performed after about 1, 2, 3, 4, 5, 6, 7, 8, 9, 10, 11, 12, 13, 14, 15, 16, 17, 18, 19, or 20 days, or about 3, 4, 5, 6, 7, or 8 weeks, or about 3, 4, 5, or 6 or more months after administration of a dose and before a next dose, if any, is administered; and/or
wherein assessment is performed after about 1, 2, 3, 4, 5, 6, 7, 8, 9, 10, 11, 12, 13, 14, 15, 16, 17, 18, 19, or 20 doses are administered.

47. The method of any one of the preceding claims, wherein onset of and/or severity of a symptom of Huntington's disease in a subject is delayed and/or reduced, and/or wherein the subject improves in one or more functional assessments.

48. The method of any one of the preceding claims, wherein an improvement is compared to baseline, absence of WVE-003 administration or administration of a reference composition, optionally wherein the reference composition is comparable to an administered WVE-003 composition but does not contain WVE-003.

49. The method of any one of the preceding claims, wherein WVE-003 is administered intrathecally and/or by direct lumbar injection.

50. The method of any one of the preceding claims, wherein a dose is administered as WVE-003 pentadecasodium salt dissolved in aCSF, optionally wherein a dose is administered in a 20 mL aCSF solution, optionally wherein 20 mL CSF is taken out from a subject before administration of a dose.

51. The method of any one of the preceding claims, wherein the subject is about 25 years old or older and/or the subject is about 60 years old or younger; and/or wherein the subject is with early manifest Huntington's disease.

52. The method of any of the previous claims, wherein the subject receives or is exposed to an additional therapeutic agent.

53. The method of any of the previous claims, wherein the subject is administered a steroid at least about one month prior to the first dose of WVE-003.

54. A composition comprising WVE-003.

55. A composition comprising WVE-003 or a composition thereof, wherein WVE-003 or the composition thereof is in a solid form and/or lyophilized.

56. A composition comprising WVE-003 or a composition thereof, wherein WVE-003 or the composition thereof is present in a vial in an amount of about 20 mg, optionally wherein the vial is backfilled with nitrogen, optionally wherein the amount of WVE-003 includes the amount of one or more pharmaceutically acceptable salt forms, each of which is independently converted to the amount of the acid form.

57. A composition comprising WVE-003 or a composition thereof, wherein WVE-003 or the composition thereof is diluted with a solution of sodium chloride, optionally wherein the solution is 0.9% sodium chloride.

58. A composition comprising WVE-003 or a composition thereof, wherein the composition consists essentially of WVE-003 or a composition thereof, sodium chloride, and water.

59. A composition comprising WVE-003 or a composition thereof, wherein the composition consists essentially of WVE-003 or a composition thereof and aCSF.

60. The composition of any one of the preceding claims, wherein a form of WVE-003 in the composition is a pharmaceutically acceptable salt form and/or WVE-003 pentadecasodium salt, or each form of WVE-003 in the composition is independently a salt form, optionally a pharmaceutically acceptable salt form and/or WVE-003 pentadecasodium salt.

61. The composition of any one of the preceding claims, wherein the composition is a drug substance and/or drug product.

62. The composition of any one of the preceding claims, wherein the composition is a liquid composition wherein WVE-003 is dissolved or wherein the composition is a pharmaceutical composition further comprising a pharmaceutically acceptable carrier, optionally wherein the pharmaceutically acceptable carrier is or comprises artificial cerebrospinal fluid (aCSF).

63. The composition of any one of the preceding claims, wherein the composition is isotonic and/or wherein the composition has a pH of about 6-8, optionally wherein the composition has a pH of about 6.4-7.2 or about 7.3 or about 7.4.

64. The composition of any one of the preceding claims, wherein the composition is lyophilized WVE-003 powder.

65. The composition of any one of the preceding claims, wherein WVE-003 in the composition is equivalent to about 5, 10, 20, 30, 40, 50, 60, 70, 80, 90, 100, 110, 120, 130, 140, 150, 160, or 170 mg WVE-003 free acid form.

66. The composition of any one of the preceding claims, wherein the composition is packaged into a vial.

67. The composition of any one of the preceding claims, wherein the composition is reconstituted and diluted in artificial cerebrospinal fluid (aCSF).

68. The composition of any one of the preceding claims, wherein the composition is a WVE-003 drug product, optionally wherein WVE-003 drug product is lyophilized WVE-003 pentadecasodium salt.

69. A vial comprising a composition of any one of the preceding claims, optionally wherein the vial is filled with an inert gas, optionally wherein the inert gas is nitrogen.

70. A syringe comprising a composition of any one of the preceding claims, optionally wherein the composition is a liquid composition wherein WVE-003 or WVE-003 pentadecasodium salt is dissolved in aCSF.

71. The syringe of claim 70, wherein the volume of the liquid composition is 20 mL.

72. The syringe of any one of claims 70-71, wherein the syringe contains a WVE-003 dose described in any one of the preceding claims.

73. A method for manufacturing a WVE-003 composition according to a method described in the specification.

74. The method of claim 73, comprising utilizing IP-RP-UPLC to assess purity and/or impurities in the manufactured WVE-003 composition and release the preparation if the purity and/or impurities meet certain criteria.

75. The method of any one of claims 73-74, wherein the composition is a drug substance or drug product.

76. A method for releasing a WVE-003 preparation, comprising utilizing IP-RP-UPLC to assess purity and/or impurities in the WVE-003 preparation and release the preparation if the purity and/or impurities meet certain criteria; or
a method for assessing purity of WVE-003 utilizing IP-RP-UPLC.

77. The method of any one of claims 74-76, wherein the IP-RP-UPLC utilized one or more parameters described in the specification and/or one or more parameters of Set A.

78. A method for confirming stereochemical identity of WVE-003 utilizing IP-RP-UPLC.

79. The method or composition of any one of the preceding claims, wherein a WVE-003 drug substance is manufactured by a process described herein, characterized by one or more methods described herein, released by one or more methods described herein, and/or stored by one or more methods described herein.

80. The method or composition of any one of the preceding claims, wherein the WVE-003 drug substance is pentadecasodium salt.

81. The method or composition of any one of the preceding claims, wherein a WVE-003 drug product is manufactured by a process described herein, characterized by one or more method described herein, released by one or more method described herein, stored by one or more method described herein, or
wherein a pharmaceutical composition is manufactured by a process described herein, characterized by one or more method described herein, released by one or more method described herein, stored by one or more method described herein.

82. The method or composition of any one of the preceding claims, wherein the composition has a purity of about 80%, 81%, 82%, 83%, 84%, 85%, 86%, 87%, 88%, 89%, 90% or more; wherein impurities in the composition are no more than about or about 20%, 19%, 18%, 17%, 16%, 15%, 14%, 13%, 12%, 11%, or 10%; and/or wherein stereochemical purity of WVE-003 is about 80%, 83%, or more.

83. The method or composition of any one of the preceding claims, wherein the purity and/or impurities are measured by IP-RP-UPLC using area % at 260 nm, optionally using % at 260 nm and the Set A parameters; and/or wherein the amount of WVE-003 is measured by UV at 260 nm and 25.0 OD/mg.

84. The method or composition of any one of the preceding claims, wherein stereochemical identity of WVE-003 is confirmed by IP-RP-UPLC, optionally by IP-RP-UPLC according to Set B parameters, or an IP-RP-UPLC method for stereochemical identity as described herein.

85. The method or composition of any one of the preceding claims, wherein stereochemical purity is assessed by dimer modeling and/or wherein stereochemical purity of WVE-003 is about 80%, 81%, 82%, 83%, 84%, or 85% or more.

86. The method or composition of any one of the preceding claims, wherein about is ±1%, ±2%, ±3%, ±4%, ±5%, ±6%, ±7%, ±8%, ±9%, or 10%.

87. A method or composition of any one of Embodiments 1-433.
